(19) United States
(12) Patent Application Publication
(10) Pub. No.: US 2026/0240794 A1
(43) Pub. Date: Aug. 20, 2026

(54) USE OF NADOLOL TO TREAT PULMONARY SYMPTOMS ASSOCIATED WITH INFECTIONS WITH SARS-COV-2

(71) Applicant: Chronic Airway Therapeutics Limited, East Kew VIC (AU)

(72) Inventor: Mitchell Glass, Wilmington, DE (US)

(21) Appl. No.: 19/164,987

(22) PCT Filed: Mar. 13, 2024
(86) PCT No.: PCT/IB2024/000152

§ 371 Date: Sep. 12, 2025

Related U.S. Application Data
(60) Provisional application No. 63/451,818, filed on Mar. 13, 2023.

(51) Int. Cl. A61K 31/138; A61K 31/137; A61K 31/403; A61K 31/522; A61K 31/5377; A61P 11/00; A61P 31/14

(52) U.S. Cl. CPC A61K 31/138; A61K 31/137; A61K 31/403; A61K 31/522; A61K 31/5377; A61P 11/00; A61P 31/14

(57) ABSTRACT

The present invention is directed to compositions and methods for treating infection with SARS-CoV-2 virus and its sequelae through inhibition of the β-arrestin (arrestin-2) pathway by use of β-adrenergic inverse agonists, particularly including nadolol. The compositions and methods can also employ additional agents to block infection with SARS-CoV-2 virus or inhibit inflammation, particularly inflammation affecting the respiratory tract.

### CROSS-REFERENCE TO RELATED APPLICATION

This application claims the benefit of U.S. Provisional Patent Application Ser. No. 63/451,818, filed Mar. 13, 2023, by Dr. Mitchell Glass, and entitled “Use of Nadolol to Treat COVID-19,” the contents of which are incorporated herein by this reference.

### FIELD OF THE INVENTION

This invention is directed to the use of nadolol or derivatives and analogues thereof to treat the viral infection COVID-19 and its sequelae, including methods and compositions employing nadolol or derivatives and analogues thereof, along with additional agents in some alternatives, to treat COVID-19 and its sequelae. In particular, this invention is directed to the use of nadolol or derivatives or analogues thereof to treat pulmonary symptoms associated with infection by SARS-CoV-2.

### BACKGROUND OF THE INVENTION

COVID-19 (coronavirus disease 2019) is a highly contagious disease caused by a virus, the severe acute respiratory syndrome coronavirus 2 (SARS-CoV-2). SARS-CoV-2 is a strain of coronavirus related to other coronaviruses that cause human disease, including SARS-CoV-1 that caused an outbreak of severe human respiratory disease (“SARS”) in 2002-2004, particularly in Asia, with cases spreading throughout the world, and an outbreak of Middle East respiratory syndrome (“MERS”) in 2012-2015, which originated in the Middle East (especially Saudi Arabia and Qatar) and subsequently spread to other locations. Other coronaviruses also cause human disease, particularly mild upper respiratory diseases such as coryza, but generally do not cause severe human disease. Coronaviruses constitute the subfamily Orthocoronavirinae in the family Coronaviridae, order Nidovirales, and realm Riboviria. They are enveloped viruses with a positive-sense single-stranded RNA genome (which can therefore act directly as mRNA in the infected cell) and a nucleocapsid with helical symmetry. The genome size of coronaviruses ranges from 26 to 32 kilobases and is among the largest genome sizes for RNA viruses. The nucleocapsid of coronaviruses, including of SARS-CoV-2, is characterized by the presence of club-shaped spikes that project from their surface, which in electron microscopy create an image reminiscent of the solar corona, giving rise to the name of the viruses. Each SARS-CoV-2 virion is 60-140 nm in diameter. Like other coronaviruses, SARS-CoV-2 has four structural proteins, known as the S (spike), E (envelope), M (membrane), and N (nucleocapsid) proteins; the S, E, and M proteins together create the viral envelope. Coronavirus S proteins are glycoproteins and also type I membrane proteins (membranes containing a single transmembrane domain oriented toward the extracellular side). Coronavirus S proteins are divided into two functional parts (S1 and S2). In the SARS-CoV-2 virus, the S protein is the protein responsible for allowing the virus to attach to and fuse with the membrane of the host cell that it infects. Specifically, the S1 subunit of the S protein catalyzes attachment and the S2 subunit catalyzes fusion.

For the SARS-CoV-2 virus, the genome is a linear, positive-sense, single-stranded RNA genome with about 30,000 bases. The genome has a bias against cytosine (C) and guanine (G) ribonucleotides, similar to other coronaviruses. The ratio of the specific bases is 32.2% U; 29.9% A; 19.6% G; and 18.3% C. This nucleotide bias arises from the mutation of guanine and cytosine nucleotides to adenines and uracils, respectively. The mutation of CG nucleotides is believed to arise to avoid the zinc finger antiviral protein-related cellular defense mechanism, and also to lower the energy required to unwind the genome during replication and translation, as A-U base pairs involve two hydrogen bonds, while C-G base pairs involve three hydrogen bonds. This depletion of CG dinucleotides in the genome leads to codon usage bias in encoding amino acids that can be encoded by multiple trinucleotide alternatives.

For a virus recently acquired via cross-species transmission, rapid evolution of the virus is expected and, in fact, has occurred during the course of the pandemic as is further addressed below. The mutation rate estimated from early cases of SARS-CoV-2 was 6.54×10⁻⁴ per site per year. Even though the evolution of SARS-CoV-2 may be slowed somewhat by the RNA proofreading capacity of its replication machinery, the likelihood of mutation occurring in vivo still remains high.

The SARS-CoV-2 virus was first detected in Wuhan, China in December 2019 and thereafter spread rapidly throughout most of the world, creating a pandemic which is still ongoing. It is presumed that the virus is of zoonotic origin and has close genetic similarity to coronaviruses that regularly infect bats, suggesting that it emerged from a bat-borne virus, possibly originally infecting humans through an intermediate host, possibly civet cats or pangolins.

Organisms other than humans can be infected by SARS-CoV-2, including, but not necessarily limited to, cats, ferrets, hamsters, non-human primates, minks, tree shrews, raccoon dogs, fruit bats, and rabbits.

Epidemiological studies during the initial course of the spread of infection in humans indicated that, in the December 2019-September 2020 period, each infection resulted in an average of 2.4 to 3.4 new infections when immunity was not present in the community and no preventative measures were taken. These estimates were obtained with the original strain of the virus. As detailed below, subsequent strains of the virus are substantially more infectious than was the original strain of the virus. The virus primarily spreads from person to person through close contact and via aerosols and respiratory droplets that are produced when infected individuals are talking, breathing, or otherwise exhaling, as well as by coughing or sneezing. The virus enters human cells by binding to angiotensin-converting enzyme 2 (ACE2), a membrane protein that is involved in regulation of the renin-angiotensin system. The virus may also use the protein basigin (CD147) to assist in cellular entry. Initial spike protein priming by transmembrane protein, serine 2 (TMPRSS2) is essential for entry of the virus. The host protein neuropilin 1 (NRP1) may also aid entry of the virus using ACE2. After a SARS-CoV-2 virion attaches to a target cell, the cellular protein TMPRSS2 cuts open the spike protein of the virus, exposing a fusion peptide in the S2 subunit, and the host receptor ACE2. After fusion, an endosome forms around the virion, separating it from the rest of the host cell. The virion escapes when the pH of the endosome drops or when the cellular cysteine protease cathepsin cleaves it. The virion then releases its RNA into the cell and causes the cell to produce and disseminate copies of the virus, which then infect more cells. The lifecycle of the SARS-CoV-2 virus is strictly a lytic lifecycle and there is no reverse transcription or incorporation of the viral RNA into the genome of host cells. During human-to-human transmission, between 200 to 800 virions are thought to be sufficient to initiate a new infection.

The degree to which the virus is infectious during its initial incubation period is uncertain. However, research has indicated that the pharynx reaches peak viral load approximately 4 days after infection or in the first week of symptoms and then declines thereafter; this period to reach peak viral load may vary with subsequent strains of the virus. The duration of virus shedding can vary broadly from infected individual to infected individual, but is generally from 3 to as much as 46 days after the onset of symptoms. These parameters of infectivity are likely to vary significantly between strains and also between subvariants of the same strain.

The nasal cavity is generally the dominant initial site of infection, with subsequent aspiration-mediated seeding of the virus into the lungs in SARS-CoV-2 viral pathogenesis. There is typically an infection gradient from high in proximal to low in distal pulmonary epithelial cultures, with focal infection in ciliated cells and type 2 pneumocytes in the airway and alveolar regions respectively. The degree of lung involvement varies with the particular strain of the virus.

Although the pathophysiology of infection by the SARS-CoV-2 virus is still being analyzed, general patterns have emerged, although it is clear that these patterns can vary substantially from variant to variant, including the cell types or tissue types that are preferentially infected. The SARS-CoV-2 virus can infect a wide variety of cells, tissues, organs, and organ systems. The SARS-CoV-2 virus is most known for infecting the upper respiratory tract, including the sinuses, nose, and throat, and the lower respiratory tract, including the windpipe and lungs. Typically, in most infections, the lungs are the organs most affected by infection by the SARS-CoV-2 virus because the virus accesses host cells through the ACE2 receptor, which is most abundant on the surface of type II alveolar cells of the lungs. Following viral entry, the SARS-CoV-2 virus infects the ciliated epithelium of the nasopharynx and upper airways. Autopsies of people who died of infection with the virus have found diffuse alveolar damage, and lymphocyte-containing inflammatory infiltrates within the lung.

The SARS-CoV-2 virus can also infect the nervous system and causes a variety of symptoms associated with infection of the cells of the nervous system. One common symptom is loss of taste or smell. This symptom typically results from infection of the support cells of the olfactory epithelium, with consequent damage to olfactory neurons. Moreover, many patients infected with the SARS-CoV-2 virus exhibit neurological or mental health issues. The exact mechanism of causation of these neurological or mental health issues is unclear. However, it has been suggested that the virus may invade the CNS by a mechanism associated with the invasion of peripheral nerves given the low levels of ACE2 in the brain; alternatively, the virus may enter the bloodstream from the lungs and cross the blood-brain barrier to gain access to the CNS, possibly via an infected leukocyte.

The SARS-CoV-2 virus also affects tissues and organs of the gastrointestinal tract, as ACE2 is abundantly expressed in the glandular cells of the gastric, duodenal, and rectal epithelium. Gastrointestinal symptoms experienced by patients infected with SARS-CoV-2 virus can include nausea, vomiting, and diarrhea.

The SARS-CoV-2 virus also frequently affects the cardiovascular system, causing acute myocardial injury and chronic damage. Acute cardiac injury is more common in severe disease. The rates of cardiovascular symptoms following infection with the SARS-CoV-2 virus are high, as a result of the systemic inflammatory response and immune system disorders during disease progression; acute myocardial injury may be associated with cardiac ACE2 receptors, which are highly expressed in cardiac tissue. Thrombosis and venous thromboembolism frequently occur in patients in intensive care units with SARS-CoV-2 virus infections, and may be related to poor prognosis; blood vessel dysfunction and clot formation, as suggested by high D-dimer levels caused by blood clots, may play a significant role in mortality of SARS-CoV-2 virus-infected patients. Additionally, incidence of clots leading to pulmonary embolisms, and ischemic events within the brain has been have been noted as complications leading to fatalities. Infection appears to set off a chain of vasoconstrictive responses within the body, including constriction of blood vessels within the pulmonary circulation. The SARS-CoV-2 virus may also cause morphological or mechanical changes to blood cells.

Infection with the SARS-CoV-2 virus also has profound immunopathological consequences. In particular, people with severe COVID have symptoms of hyperinflammation. Clinical laboratory findings of elevated IL-2, IL-7, IL-6, granulocyte-macrophage colony-stimulating factor (GM-CSF), interferon gamma-induced protein 10 (IP-10), monocyte chemoattractant protein 1 (MCP1), macrophage inflammatory protein 1-alpha (MIP-1-alpha), and tumor necrosis factor (TNF-α) indicative of cytokine release syndrome (CRS), sometimes referred to as “cytokine storm,” suggest an underlying immunopathology.

Additionally, infection with the SARS-CoV-2 virus can produce acute respiratory distress syndrome (ARDS); such patients have classical serum biomarkers of CRS, including elevated C-reactive protein (CRP), lactate dehydrogenase (LDH), D-dimer, and ferritin. Systemic inflammation results in vasodilation, allowing inflammatory lymphocytic and monocytic infiltration of the lung and heart. In particular, pathogenic GM-CSF-secreting T cells were shown to correlate with the recruitment of inflammatory IL-6-secreting monocytes and also correlate with severe lung pathology in infected patients.

As stated above, rapid evolution of the virus was expected in view of the life cycle and reproductive mechanism of the virus and has occurred during the course of the pandemic. The original strain of the virus has almost universally been succeeded by multiple variants. These variants typically include mutations in the spike protein. The variants include: Alpha, Beta, Gamma, Delta, and Omicron; several of these variants, particularly Omicron, have given rise to one or more subvariants. The Alpha variant, lineage B.1.1.7, emerged in the United Kingdom in September 2020, with evidence of increased transmissibility and virulence; its notable mutations include N501Y and P681H. The Beta variant, lineage B.1.351, emerged in South Africa in May 2020, with evidence of increased transmissibility and changes to antigenicity, and the possibility of reduced efficacy of vaccines; notable mutations include K417N, E484K, and N501Y. The Gamma variant, lineage P.1, emerged in Brazil in November 2020, also with evidence of increased transmissibility and virulence, along changes to antigenicity; concerns about the possibility of reduced efficacy of vaccines have also been raised; notable mutations include K417N, E484K, and N501Y. The Delta variant, lineage B.1.617.2, emerged in India in October 2020; there is evidence of increased transmissibility and changes to antigenicity. The Omicron variant, lineage B.1.1.529, emerged in Botswana in November 2021. In general, the Omicron variant is considered to be substantially more transmissible than the original strain and the previously-emerged variants, although it is not considered to cause more severe disease than either the original strain or the previously-emerged variants. Several subvariants of the Omicron variant have recently emerged which are considered to be still more transmissible than the original Omicron variant, although they are not thought to cause more serious disease than either the original strain or the original Omicron variant. These subvariants include BA.1, BA.2, BA.3, BA.4, BA.5, BQ.1, BQ.1.1, XE, BA.2.12, BA.2.75, BA.2.75.2, XBB, XBB.1, HV.1, EG.5, EG.5.1, and BA.2.86. Further variants may emerge; it is believed that a major source of variants is infection of immunocompromised patients, such as patients with AIDS, transplant patients being treated with immunosuppressant drugs such as cyclosporin, or cancer patients being treated with immunosuppressant anti-neoplastic drugs such as alkylating agents; in such patients, the period of replication of the virus can be substantially prolonged, leading to the opportunity for imprecise replication of the virus, resulting in variants with mutations at one or more positions in the genome. Therefore, it is likely that additional subvariants of Omicron will continue to emerge. In particular, the infectivity of a number of the Omicron subvariants has been evaluated as being comparable to the infectivity of the measles virus, which is generally considered to be the most infectious virus commonly affecting humans. Although Omicron and its subvariants are also generally considered to cause somewhat milder disease than the original strain, they do cause considerable respiratory illness which may focus on the upper respiratory tract rather than the lungs. Additionally, the mRNA vaccines in general use are considered to provide reduced protection against asymptomatic illness but do reduce the risk of serious illness.

As of Mar. 10, 2023, the pandemic caused by infection with the SARS-CoV-2 virus has caused more than 676 million diagnosed cases and 6.88 million confirmed deaths, making this one of the most deadly pandemics in recorded history. These numbers are estimates and are believed to be substantial undercounts, as many infections, particularly with the Omicron strain and its subvariants, are either asymptomatic or mild, and many deaths in patients infected with SARS-CoV-2 have likely been attributed to other causes.

The symptomatology of infection with the SARS-CoV-2 virus can vary widely, depending on a number of factors, including, but not limited to, the particular strain or subvariant of the virus infecting the patient, the vaccination status of the patient, including whether or not the patient has been vaccinated with booster vaccinations, the number of booster vaccinations administered, and the time interval between the most recent booster vaccination and infection, and the medical status of the patient. Many patients are completely asymptomatic. For others, symptoms can range from symptoms generally considered characteristic of coryza (the “common cold”) such as a runny nose, sneezing, coughing, and sore throat, through symptoms generally considered characteristic of influenza, such as fever, body aches, and fatigue, up to more severe symptoms such as pneumonia and shortness of breath, which may necessitate hospitalization in an intensive care unit and use of a respirator. Patients with severe infections with SARS-CoV-2 virus are likely to develop acute respiratory distress syndrome (ARDS), consisting of hypoxemic respiratory failure associated with neutrophilia, mucus deposition in bronchi, and bronchiectasis. In particular, there are a number of diseases and conditions that can affect the symptomatology of infection with the SARS-CoV-2 virus and the eventual outcome of the infection. These diseases or conditions include, but are not limited to: obesity; smoking; high blood pressure; diabetes; immunodeficiency, whether occurring as the result of infection with HIV or as the result of administration of drugs that depress the functioning of the immune system, such as anti-neoplastic drugs, particularly alkylating agents, as well as drugs that are administered to prevent graft rejection or the occurrence of graft-versus-host disease; and the general health of the patient. Patients with one or more of these diseases or conditions are more likely to experience worse outcomes as compared with patients who are free of these conditions. Additionally, age is also a factor with respect to the symptomatology and eventual outcome of the infection; elderly patients tend to have more severe disease and worse outcomes. In particular, patients who are 85 years of age or older are at particular risk of more severe disease and of death.

A number of types of vaccines have been authorized for use against SARS-CoV-2 infection. These vaccines include: mRNA vaccines, which typically contain mRNA encoding the SARS-Cov-2 spike protein; adenovirus-based vaccines, which use an adenovirus shell containing DNA that encodes a SARS-Cov-2 protein; inactivated vaccines that consist of virus particles that are grown in culture and then inactivated using a method such as heat and formaldehyde to lose disease-producing capacity while still inducing an immune response; subunit vaccines, which present one or more antigens without introducing entire virions; virus-like particle vaccines; DNA plasmid vaccines; lentivirus vector vaccines; conjugate vaccines; and vesicular stomatitis virus vaccines displaying the SARS-CoV-2 spike proteins. Although the vaccines in current use, particularly the mRNA-based vaccines, are highly effective in preventing serious illness, hospitalization, and death, they are significantly less effective in preventing infection, even in people who have been vaccinated and boosted. Moreover, the immunity produced by the vaccines in current use tends to wane over a period of several months, leading to the requirement for boosters to be administered to keep immunity at a suitable level. Additionally, there is a likelihood that, because most vaccines in use are based on the immunogenicity of the spike protein, the development of further variants or subvariants which may have significant mutations in the spike protein would reduce the effectiveness of such vaccines. In addition, vaccination in patients with immune deficiencies typically results in a weak immune response to the vaccine.

Diagnosis of infection with SARS-CoV-2 is typically done by either nucleic acid tests, which determine the existence of specific SARS-CoV-2 RNA, or by antigen tests, which determine the existence of a SARS-CoV-2 protein; this protein is typically a protein from the surface spikes of the virus.

Treatment of infections caused by SARS-CoV-2 virus is being actively researched; however, at this point, a limited number of specific treatment options are available. Patients with relatively mild to moderate symptoms benefit from a combination of nirmatrelvir and ritonavir (marketed as a single pill as Paxlovid®), particularly when administered within a few days of confirmation of the diagnosis. Remdesivir, another antiviral agent, has also been employed, but again is particularly useful in patients in which the drug is administered within a few days of confirmation of the diagnosis. Molnupiravir, yet another antiviral agent, which inhibits viral replication by causing replication errors in the replication reaction catalyzed by RNA-directed RNA polymerase, is also potentially useful in patients with a high risk of severe disease. The “cytokine storm” may be treatable by administration of anti-inflammatory corticosteroids such as betamethasone or dexamethasone. Monoclonal antibodies have also been used as therapeutic agents, but their efficacy can be limited by their very specificity for a particular epitope on a protein of the virus, typically the spike protein, so that such monoclonal antibodies may have substantially less efficacy against subsequent variants in which the spike protein has one or more mutations. In particular, most monoclonal antibodies previously in use have been found to be substantially or completely ineffective against recently emerging subvariants of the Omicron variant.

However, there remains a substantial need for therapeutic modalities that can treat many of the severe symptoms produced by infection with SARS-CoV-2 virus or associated with the sequelae of such infection. Preferably, such therapeutic modalities would not depend for their activity on the genetic makeup of a particular variant of SARS-CoV-2 virus, could be used together with antiviral agents or other agents or with vaccines, and would be well-tolerated and produce few, if any side effects. Additionally, preferably, such therapeutic modalities can be used at both early stages of the infection and subsequent stages of the infection, when symptoms are frequently more severe. Also, preferably, such therapeutic modalities could be used to treat persistent respiratory symptoms that may occur even after the SARS-CoV-2 virus is no longer detectable in the patient or is detectable in the patient only at an extremely low level so that the patient is no longer infectious.

### SUMMARY OF THE INVENTION

The present invention is directed to the use of nadolol or derivatives or analogues of nadolol to treat infections caused by the SARS-CoV-2 virus. The use of nadolol or derivatives or analogues of nadolol to treat infections caused by the SARS-CoV-2 virus would not depend for their activity on the genetic makeup of a particular variant of SARS-CoV-2 virus, could be used together with antiviral agents or other agents, including vaccines, and would be well-tolerated and produce few, if any side effects. Nadolol or derivatives or analogues of nadolol to treat infections caused by the SARS-CoV-2 virus or sequelae of such infections can be used at both early stages of the infection and subsequent stages of the infection, when symptoms are frequently more severe. Additionally, such therapeutic modalities can be used to treat persistent respiratory symptoms that may occur even after the SARS-CoV-2 virus is no longer detectable in the patient or is detectable in the patient only at an extremely low level so that the patient is no longer infectious.

In one alternative of a method according to the present invention, the treatment is treatment of infection with SARS-CoV-2 virus. In another alternative of a method according to the present infection, the treatment is treatment of sequelae of infection with SARS-CoV-2 virus.

Typically, the method comprises administration of a therapeutically effective quantity of nadolol. Typically, the nadolol is the RSR stereoisomer of nadolol.

In one alternative, the method exerts a therapeutic effect that is an upregulation of pulmonary β₂-adrenergic receptors. Typically, the method exerts a therapeutic effect that is increased pulmonary airway relaxation responsiveness to β₂-adrenergic agonist drugs. Typically, the method exerts a therapeutic effect that is a reversal of mucous metaplasia and mucus cell hyperplasia. Typically, the method exerts a therapeutic effect that is a blockage of the ability of the SARS-CoV-2 virus to infect cells via the ACE2 receptor.

Typically, the nadolol or the derivative or analogue of nadolol is administered by a route selected from the group consisting of oral, sustained-release oral, parenteral, sublingual, buccal, administration by insufflation, and administration by inhalation. Typically, the administration of the nadolol or the derivative or analogue of nadolol is performed by dose titration over time in a series of graduated doses starting from the lowest dose and increasing to the highest dose. Typically, when the highest dose is reached, the nadolol or the derivative or analogue of nadolol continues to be administered at that dose. Preferably, the nadolol or the derivative of nadolol is administered by the inhaled route or the oral route. More preferably, the nadolol or the derivative of nadolol is administered by the inhaled route. Typically, the method of administering the nadolol by inhaled administration comprises administration of a dose administered by pressurized meter dose inhaler (pMDI), dry powder inhaler, or nebulizer in doses that either do or not generate measurable blood levels in the range typically associated with oral dosing. Typically, the inhaled dose will be delivered by pMDI and will be in the range of from about 1% to about 10% of the minimally effective oral dose.

Typically, the method of sustained-release oral administration of the nadolol or the derivative or analogue of nadolol results in continuous levels of the nadolol or the derivative or analogue of nadolol in the bloodstream.

Typically, in a method according to the present invention, the inhibition of β-arrestin prevents or reverses the desensitization of β₂-adrenergic receptors. Typically, in a method according to the present invention, the inhibition of β-arrestin also prevents or reverses the internalization of β₂-adrenergic receptors. Typically, in a method according to the present invention, the inhibition of β-arrestin prevents or reverses phosphorylation of β₂-adrenergic receptors by a second-messenger-specific protein kinase or a specific G-protein-coupled receptor kinase. Typically, in a method according to the present invention, the inhibition of β-arrestin also prevents or reverses degradation of a second messenger by a scaffolding phosphodiesterase.

In one alternative, the method further comprises administration of a therapeutically effective quantity of β₂-selective adrenergic agonist. Typically, the β₂-selective adrenergic agonist is selected from the group consisting of abediterol, arformoterol, bambuterol, bitolterol, broxaterol, carbuterol, carmoterol, clenbuterol, colterol, dobutamine, fenoterol, formoterol, indicaterol, isoprenaline, isoxsuprine, levabuterol, mabuterol, metaproterenol, methoxyphenamine, navafenterol, olodaterol, pirbuterol, procaterol, ritodrine, salbutamol, salmeterol, terbutaline, vilanterol, and zilpaterol, and the salts, solvates, and prodrugs thereof.

In another alternative, the method further comprises administration of a therapeutically effective quantity of a corticosteroid. Typically, the corticosteroid is selected from the group consisting of beclomethasone, budesonide, ciclesonide, deflazacort, flunisolide, fluticasone, methylprednisolone, mometasone, prednisolone, prednisone, betamethasone, dexamethasone, triamcinolone, acrocinonide, alclometasone, amcinafal, amcinafide, amebucort, amelometasone, benzodrocortisone, butixocort, chloroprednisone, ciclometasone, ciprocinonide, clobetasol, clorcotolone, cormetasone, cortobenzolone, deprodone, descinolone, dexbudesonide, dimesone, domoprednate, doxibetasol, drocinonide, endrisone, etiprednol, fluazacort, flucorolone, flumetasone, flumoxonide, fluocinolone, fluocinonide, fluocortin, fluocortolone, 9α-fluorocortisone, fluorometholone, flupamesone, fluperolone, fluprednidene, halcinonide, halocortolone, halopredone, halocortamate, hydrocortisone, icometasone, isoflupredone, isoprednidene, itrocinonide, locicortolone, mazipredone, meclorisone, meprednisone, naflocort, nicocortonide, nivacortol, oxisopred, paramethasone, prednylidene, procinonide, resocortol, rofleponide, ticabesone, timobesone, tipredane, tixocortol, tralonide, triclonide, and the salts, solvates, and prodrugs thereof. Preferably, the corticosteroid is selected from the group consisting of betamethasone, beclomethasone, budesonide, ciclesonide, flunisolide, fluticasone, methylprednisolone, prednisolone, prednisone, dexamethasone, and triamcinolone, and the salts, solvates, and prodrugs thereof. More preferably, the corticosteroid is selected from the group consisting of betamethasone and dexamethasone.

In yet another alternative, the method further comprises administration of a therapeutically effective quantity of an anticholinergic drug. Typically, the anticholinergic drug is a muscarinic receptor antagonist. Preferably, the muscarinic receptor antagonist is a quaternary ammonium muscarinic receptor antagonist. More preferably, the quaternary ammonium muscarinic receptor antagonist is selected from the group consisting of ipratropium bromide, tiotropium bromide, oxitropium bromide, aclidinium bromide, glycopyrronium bromide, umeclidinium bromide, and abediterol, and the salts, solvates, and prodrugs thereof.

In still another alternative, the method further comprises administration of a therapeutically effective quantity of a xanthine compound. Typically, the xanthine compound is selected from the group consisting of theophylline, extended-release theophylline, aminophylline, theobromine, enprofylline, diprophylline, isbufylline, choline theophyllinate, albifylline, arofylline, bamifylline, caffeine, 8-chlorotheophylline, diprophylline, doxofylline, enprofylline, etamiphylline, furafylline, 3-isobutyl-1-methylxanthine, IBMX (1-methyl-3-(2-methylpropyl)-7H-purine-2,6-dione), MRS-1706 (N-(4-acetylphenyl)-2-[4-(2,3,6,7-tetrahydro-2,6-dioxo-1,3-dipropyl-1H-purin-8-yl)phenoxy]acetamide), ambuphylline, proxyphylline, and xanthinol, and the salts, solvates, and prodrugs thereof. Preferably, the xanthine compound is selected from the group consisting of theophylline, extended-release theophylline, aminophylline, theobromine, enprofylline, diprophylline, isbufylline, choline theophyllinate, albifylline, arofylline, bamifylline, ambuphylline, 8-chlorotheophylline, doxofylline, furafylline, IBMX (1-methyl-3-(2-methylpropyl)-7H-purine-2,6-dione), MRS-1706 (N-(4-acetylphenyl)-2-[4-(2,3,6,7-tetrahydro-2,6-dioxo-1,3-dipropyl-1H-purin-8-yl)phenoxy]acetamide), proxyphylline, and caffeine, and the salts, solvates, and prodrugs thereof.

In yet another alternative, the method further comprises administration of a therapeutically effective quantity of an anti-IgE antibody. Typically, the anti-IgE antibody is a monoclonal antibody or a genetically engineered antibody that is derived from a monoclonal antibody; the antibody can be humanized, such as omalizumab.

In yet another alternative, the method further comprises administration of a therapeutically effective quantity of a leukotriene antagonist. Typically, the leukotriene antagonist is selected from the group consisting of ablukast, tipelukast, montelukast, pranlukast, and zafirlukast, and the salts, solvates, and prodrugs thereof.

In still another alternative, the method further comprises administration of a therapeutically effective quantity of a phosphodiesterase IV inhibitor. Typically, the phosphodiesterase IV inhibitor is selected from the group consisting of apremilast, atizoram, filaminast, mufemilast, roflumilast, cilomilast, piclamilast, and ibudilast, and the salts, solvates, and prodrugs thereof.

In yet another alternative, the method further comprises administration of a therapeutically effective quantity of a 5-lipoxygenase inhibitor. Typically, the 5-lipoxygenase inhibitor is selected from the group consisting of meclofenamate sodium, zileuton, and fenleuton, and the salts, solvates, and prodrugs thereof.

In still another alternative, the method further comprises administration of a therapeutically effective quantity of a biological. Typically, the biological is selected from the group consisting of an anti-IL4 antibody, an anti-IL-13 antibody, an inhibitor of both IL-4 and IL-13, an anti-IL-5 antibody, and an anti-IL-8 antibody.

In yet another alternative, the method further comprises administration of a therapeutically effective quantity of a MAPK inhibitor. Typically, the MAPK inhibitor is selected from the group consisting of PD 184352 (2-(2-chloro-4-iodoanilino)-N-(cyclopropylmethoxy)-3,4-difluorobenzamide), neflamapimod, SB 202190 (4-[4-(4-fluorophenyl)-5-pyridin-4-yl-1H-imidazol-2-yl]phenol hydrochloride), anisomycin, PD 98059 (2-(2-amino-3-methoxyphenyl)chromen-4-one), SB 203580 (4-[4-(4-fluorophenyl)-2-(4-methylsulfinylphenyl)-1H-imidazol-5-yl]pyridine hydrochloride), U0126 (1,4-diamino-2,3-dicyano-1,4-bis(2-aminophenylthio)butadiene), AG 126 (2-[(3-hydroxy-4-nitrophenyl)methylidene]propanedinitrile), apigenin, 5-iodotubercidin, olomoucine, iso-olomoucine, N⁹-isopropyl olomoucine, PD 169316 (4-[4-(4-fluorophenyl)-2-(4-nitrophenyl)-1H-imidazol-5-yl]pyridine), SB 202474 (4-[5-ethyl-2-(4-methoxyphenyl)-1H-imidazol-4-yl]pyridine), SB 202190 (4-[4-(4-fluorophenyl)-5-pyridin-4-yl-1H-imidazol-2-yl]phenol hydrochloride), SB 202474 (4-[5-ethyl-2-(4-methoxyphenyl)-1H-imidazol-4-yl]pyridine dihydrochloride), adezmapimod, SB 203580 sulfone (4-[4-(4-fluorophenyl)-2-(4-methylsulfonylphenyl)-1H-imidazol-5-yl]pyridine), SB 220025 (4-[5-(4-fluorophenyl)-3-piperidin-4-ylimidazol-4-yl]pyrimidin-2-amine), SC 68376 (2-methyl-4-phenyl-5-pyridin-4-yl-1,3-oxazole), SKF-86002 (6-(4-fluorophenyl)-5-pyridin-4-yl-2,3-dihydroimidazo[2,1-b][1,3]thiazole), U0124 ((2Z,3Z)-2,3-bis[amino(methylsulfanyl)methylidene]butanedinitrile), ZM 336372 (3-(dimethylamino)-N-[3-[(4-hydroxybenzoyl)amino]-4-methylphenyl]benzamide), doramapimod, ralimetinib dimesylate, PH-797804 (3-[3-bromo-4-[(2,4-difluorophenyl)methoxy]-6-methyl-2-oxopyridin-1-yl]-N,4-dimethylbenzamide), VX-702 (6-(N-carbamoyl-2,6-difluoroanilino)-2-(2,4-difluorophenyl)pyridine-3-carboxamide), losmapimod, SB 269063 (4-[4-(4-fluorophenyl)-5-(2-methoxypyrimidin-4-yl)imidazol-1-yl]cyclohexan-1-ol), BMS-582949 (4-[5-(cyclopropylcarbamoyl)-2-methylanilino]-5-methyl-N-propylpyrrolo[2,1-f][1,2,4]triazine-6-carboxamide), skepinone-L, TAK-715 (N-[4-[2-ethyl-4-(3-methylphenyl)-1,3-thiazol-5-yl]pyridin-2-yl]benzamide), pamapimod, pexmetinib, SD 0006 (1-[4-[3-(4-chlorophenyl)-4-pyrimidin-4-yl-1H-pyrazol-5-yl]piperidin-1-yl]-2-hydroxyethanone), and R1487 (6-(2,4-difluorophenoxy)-8-methyl-2-(oxan-4-ylamino)pyrido[2,3-d]pyrimidin-7-one hydrochloride), and the salts, solvates, and prodrugs thereof.

In still another alternative, the method further comprises administration of a therapeutically effective quantity of a mast cell stabilizer. Typically, the mast cell stabilizer is selected from the group consisting of bepotastine, alcaftadine, azelastine, cromoglicic acid, ketotifen, lodoxamide, nedocromil, olopatadine, and pemirolast, and the salts, solvates, and prodrugs thereof. Preferably, the mast cell stabilizer is selected from the group consisting of azelastine, cromoglicic acid, ketotifen, lodoxamide, nedocromil, olopatadine, and pemirolast, and the salts, solvates, and prodrugs thereof.

In yet another alternative, the method further comprises administration of a therapeutically effective quantity of an arrestin-2 inhibitor.

One alternative of an arrestin-2 inhibitor that can be used in methods according to the present invention is a protein fragment of arrestin-2.

Another alternative of an arrestin-2 inhibitor that can be used in methods according to the present invention is a compound of Formula (A-I) with alternatives for substituents as defined below in the Detailed Description of the Invention.

Another alternative of an arrestin-2 inhibitor that can be used in methods according to the present invention is an omega-3 fatty acid selected from the group consisting of DHA (docosahexaenoic acid), EPA (eicosapentaenoic acid), HTA (hexadecatrienoic acid), ALA (ax-linolenic acid), SDA (stearidonic acid), ETE (eicosatrienoic acid), ETA (eicosatetraenoic acid), HPA (heneicosapentaenoic acid), DPA (docosapentaenoic acid), tetracosapentaenoic acid, and tetracosahexaenoic acid.

Yet another alternative of an arrestin-2 inhibitor that can be used in methods according to the present invention is a CXCR2 inhibitor selected from the group consisting of SB225002 (N-(2-bromophenyl)-N′-(2-hydroxy-4-nitrophenyl)urea), AZD5069 (N-(2-((2,3-difluorobenzyl)thio)-6-(((2R,3S)-3,4-dihydroxybutan-2-yl)oxy)pyrimidin-4-yl)azetidine-1-sulfonamide); SB265610 (1-(2-bromophenyl)-3-(4-cyano-1H-benzo[d][1,2,3]triazol-7-yl)urea); navarixin; danirixin; CXCR2-IN-1 (1-(2-chloro-3-fluorophenyl)-3-[4-chloro-2-hydroxy-3-(1-methylpiperidin-4-yl)sulfonylphenyl]urea); SRT3109 (N-(2-((2,3-difluorobenzyl)thio)-6-((3,4-dihydroxybutan-2-yl)amino)pyrimidin-4-yl)azetidine-1-sulfonamide); and SRT3190 (N-[2-[(2,3-difluorophenyl)methylsulfanyl]-6-[[(2S,3R)-3,4-dihydroxybutan-2-yl]amino]pyrimidin-4-yl]azetidine-1-sulfonamide).

Still another alternative of an arrestin-2 inhibitor that can be used in methods according to the present invention is a MyD88 inhibitor. Typically, the MyD88 inhibitor is selected from the group consisting of ST2825 ((4R,7R,8aR)-1′-[2-[4-[[2-(2,4 -dichlorophenoxy)acetyl]aamino]phenyl]acetyl]-6-oxospiro[3,4,8,8a-tetrahydro-2H-pyrrolo[2,1-b][1,3]thiazine-7,2′-pyrrolidine]-4-carboxamide), and T6167923 (4-(3-bromophenyl)sulfonyl-N-(1-thiophen-2-ylethyl)piperazine-1-carboxamide).

Yet another alternative of an arrestin-2 inhibitor that can be used in methods according to the present invention is a MD2 inhibitor. Typically, the MD2 inhibitor is L48H37 ((3E,5E)-1-ethyl-3,5-bis[(2,3,4-trimethoxyphenyl)methylidene]piperidin-4-one).

Still another alternative of an arrestin-2 inhibitor that can be used in methods according to the present invention is inositol hexaphosphate (IP6).

Yet another alternative of an arrestin-2 inhibitor that can be used in methods according to the present invention is barbadin.

Still another alternative of an arrestin-2 inhibitor that can be used in methods according to the present invention is an inhibitor of protein kinase A. Typically, the inhibitor of protein kinase A is selected from the group consisting of: (i) H89 (N-[2-[[3-(4-bromophenyl)-2-propenyl]amino]ethyl]-5-isoquinolinesulfonamide dihydrochloride); (ii) N-(ω-undecylenoyl)phenylalanine; (iii) 3′,5′-cyclic monophosphorothioate-R; (iv) H-7 (5-(2-methylpiperazin-1-yl)sulfonylisoquinoline dihydrochloride); (v) H-9 (N-(2-aminoethyl)-5-isoquinolinesulfonamide; (vi) 6-22 amide; and (vii) a protein kinase A inhibitor selected from the group consisting of: fasudil; N-[2-(phosphorylated bromonitroarginylamino)ethyl]-5-isoquinoline sulfonamide; 1-(5-quinolinesulfonyl)piperazine; 4-cyano-3-methylisoquinoline; acetamido-4-cyano-3-methylisoquinoline; 8-bromo-2-monoacyladenosine-3,5-cyclic monophosphorothioate; adenosine 3,5-cyclic monophosphorothioate; 2-O-monobutyl-cyclic adenosine monophosphate; 8-chloro-cyclic adenosine monophosphate; N-[2-(cinnamoylamino acid)]-5-isoquinolinone; reverse phase-8-hexylamino adenosine 3,5-monophosphorothioate; reverse phase-8-piperidinyladenosine-cyclic adenosine monophosphate; reverse phase-adenosine 3,5-cyclic monophosphorothioate; 5-iodotuberculin; 8-hydroxyadenosine-3,5-monophosphorothioate; calphostin C; daphnetin; reverse phase-8-chlorophenyl-cyclic adenosine monophosphate; reverse phase-cyclic adenosine monophosphate; reverse phase-8-Br-cyclic adenosine monophosphate; 1-(5-isoquinolinesulfonyl)-2-methylpiperidine; 8-hydroxyadenosine-3′,5′-monophosphate; 8-hexylaminoadenosine-3′,5′-monophosphate; and reverse phase-adenosine 3′,5′-cyclic monophosphate.

Yet another alternative of an arrestin-2 inhibitor that can be used in methods according to the present invention is a phospholipase C inhibitor. Typically, the phospholipase C inhibitor is selected from the group consisting of sodium aristolochate; D609 (sodium tricyclodecan-9-yl xanthogenate); D-erythro-dihydrosphingosine; U-73122 (1-(6-((17β-3-methoxyestra-1,3,5(10)-trien-17-yl)amino)hexyl)-1H-pyrrole-2,5-dione); pyrrolidinethiocarbamate; neomycin sulfate; thielavin B; edelfosine; heterocyclyl-substituted anilino phospholipase C inhibitors; DCIC (3,4-dichloroisocoumarin); and calporoside or derivatives of calporoside.

In still another alternative, the method further comprises administration of a therapeutically effective quantity of an inhibitor of a GRK. Typically, the inhibitor of the GRK is a nitric oxide donor that donates nitric oxide or a related redox species. Alternatively, the inhibitor of the GRK can be selected from the group consisting of paroxetine and Cmpd101 (3-[(4-methyl-5-pyridin-4-yl-1,2,4-triazol-3-yl)methylamino]-N-[[2-(trifluoromethyl)phenyl]methyl]benzamide). The inhibitor of the GRK indirectly acts as an inhibitor of arrestin-2.

In yet another alternative, the additional agent is an anti-SARS-CoV-2 virus agent.

The anti-SARS-CoV-2 virus agent, in one alternative, is a vaccine. Typically, the vaccine is selected from the group consisting of: an mRNA-based vaccine; an adenovirus-based vaccine; an inactivated vaccine consisting of virus particles that are grown in culture and then inactivated; a subunit vaccine; a virus-like particle vaccine; a DNA plasmid vaccine; a lentivirus vector vaccine; a conjugate vaccine; and a vesicular stomatitis vaccine displaying the SARS-CoV-2 virus spike protein.

In another alternative, the additional agent is an agent for treating a SARS-CoV-2 infection. The agent for treating a SARS-CoV-2 infection can be selected from the group consisting of a monoclonal antibody and a small molecule. When the agent for treating a SARS-CoV-2 infection is a monoclonal antibody, the monoclonal antibody can be selected from the group consisting of: (i) monoclonal antibodies that block the infection of susceptible cells by the SARS-CoV-2 virus; and (ii) monoclonal antibodies that act by other mechanisms. Typically, the antigen of the SARS-CoV-2 virus to which the monoclonal antibody binds is an epitope of the spike protein. The monoclonal antibody can be selected from the group consisting of bebtelovimab, bamlanivimab, etesevimab, casirivimab, imdemivab, tixagevimab, cilgavimab, regdanvimab, sotrovimab, vilobelimab, sarilumab, lenzilumab, and levilimab. When the agent for treating a SARS-CoV-2 infection is a small molecule, the small molecule can be selected from the group consisting of a fixed-dose combination of nirmatrelvir and ritonavir; a combination of nirmaltrevir and a CYP3A4 inhibitor selected from the group consisting of boceprevir, indinavir, nelfinavir, saquinavir, clarithromycin, telithromycin, ceritinib, mibefradil, nefazodone, ribociclib, tucatinib, chloramphenicol, ketoconazole, itraconazole, posaconazole, voriconazole, and cobicistat; remdesivir; favipravir; molnupravir; T-1105 (2-oxo-1H-pyrazine-3-carboxamide); T-1106 (4-[(2R,3R,4S,5R)-3,4-dihydroxy-5-(hydroxymethyl)oxolan-2-yl]-3-oxopyrazine-2-carboxamide); lufotrelvir; merafloxacin; olgotrevir; ASC-09 ([(3aS,4R,6aR)-2,3,3a,4,5,6a-hexahydrofuro[2,3-b]furan-4-yl]N-[(2S,3R)-4-[[2-[(1-cyclopentylpiperidin-4-yl)amino]-1,3-benzothiazol-6-yl]sulfonyl-(2-methylpropyl)amino]-3-hydroxy-1-phenylbutan-2-yl]carbamate); danoprevir; deuremidevir; disulfiram; ensitrelvir; simnotrelvir; talfirastide; and tipiracil. Alternatively, the small molecule can be: apabetalone; an inhibitor of TMPRSS2 that is typically selected from the group consisting of camostat, nafamostat, bromhexine, and N-0385 ((2S)—N-[(2S)-1-[[(2S)-1-(1,3-benzothiazol-2-yl)-5-(diaminomethylideneamino)-1-oxopentan-2-yl]amino]-1-oxo-3-phenylpropan-2-yl]-2-(methanesulfonamido)pentanediamide); an agent that blocks the activity of human protein eEF1A that is typically plitidepsin; a selective serotonin reuptake inhibitor that is typically selected from the group consisting of fluoxetine and fluvoxamine; an anti-mitotic agent that is typically selected from the group cabazitaxel, docetaxel, epothilone, ixabepilone, larotaxel, neoxaline, ortataxel, paclitaxel, sabizabulin, tesetaxel, demecolcine, vinblastine, nocodazole, vincristine, vindesine, vinflunine, and vinorelbine; an agent that is a SGLT2 inhibitor that is typically selected from the group consisting of canagliflozin, dapagliflozin, empagliflozin, ertugliflozin, ipragliflozin, luseogliflozin, remogliflozin etabonate, sergliflozin etabonate, sotagliflozin, and tofogliflozin; an IL-6 inhibitor that is typically selected from the group consisting of sarilumab and siltuximab; an IL-1 inhibitor that is typically selected from the group consisting of anakinra and canakinumab; a JAK pathway inhibitor that is typically tofacitinib; a TNF-α inhibitor that is typically infliximab; an IL-17 inhibitor that is typically secukinumab; an agent that inhibits MUC5AC that is typically selected from the group consisting of niclosamide and carbocisteine; an EGFR tyrosine kinase inhibitor that is typically selected from the group consisting of gefitinib, afatinib, brigatinib, erlotinib, lapatinib, neratinib, olmutinib, osimertinib, rociletinib, and vandetanib; a p38 mitogen-activated protein kinase inhibitor that is typically acumapimod; an anti-IDO antibody, clone 10.1; a statin that is typically selected from the group consisting of simvastatin, atorvastatin, fluvastatin, lovastatin, pitavastatin, pravastatin, and rosuvastatin; and BIO-11006 acetate.

Another aspect of the present invention is a pharmaceutical composition comprising:
(1) a therapeutically effective quantity of nadolol or a derivative or analogue of nadolol to inhibit the β-arrestin pathway to treat infection with SARS-CoV-2 virus or sequelae of such infection; and
(2) a pharmaceutically acceptable carrier.

Typically, the pharmaceutical composition comprises a therapeutically effective quantity of nadolol. Alternatively, the pharmaceutical composition comprises a therapeutically effective quantity of a derivative or analogue of nadolol.

Typically, administration of the pharmaceutical composition exerts a therapeutic effect that is an upregulation of pulmonary β₂-adrenergic receptors. Typically, administration of the pharmaceutical composition exerts a therapeutic effect that is increased pulmonary airway relaxation responsiveness to β₂-adrenergic agonist drugs. Typically, administration of the pharmaceutical composition exerts a therapeutic effect that is a reversal of mucous metaplasia, mucus cell hyperplasia, or both mucous metaplasia and mucus cell hyperplasia. Typically, administration of the composition exerts a therapeutic effect that is a blockage of the ability of the SARS-CoV-2 virus to infect cells via the ACE2 receptor.

Typically, the pharmaceutical composition is formulated for administration by a route selected from the group consisting of oral, sustained-release oral, parenteral, sublingual, buccal, administration by insufflation, and administration by inhalation. Preferably, the pharmaceutical composition is formulated for administration by inhalation, oral administration, or sustained-release oral administration.

In one alternative, when the pharmaceutical composition is formulated for administration by inhalation and the nadolol or derivative or analogue of nadolol is nadolol, administration of the composition produces evanescent blood levels of nadolol or produces no detectable blood levels of nadolol.

In another alternative, when the composition is formulated for sustained-release oral administration, administration of the pharmaceutical composition results in continuous levels of the nadolol or the derivative or analogue of nadolol in the bloodstream.

In another alternative, when the nadolol or derivative or analogue of nadolol is nadolol, the quantity of nadolol per unit dose of the composition is selected from the group consisting of 1 mg, 3 mg, 5 mg, 10 mg, 15 mg, 30 mg, 50 mg, and 70 mg per unit dose.

In yet another alternative, the composition comprises a therapeutically effective quantity of an additional therapeutic agent. Suitable additional therapeutic agents are as described above with respect to methods according to the present invention.

In one alternative of a composition comprising an additional therapeutic agent, the additional therapeutic agent is a β₂-selective adrenergic agonist as described above.

In another alternative of a composition comprising an additional therapeutic agent, the additional therapeutic agent is a corticosteroid as described above.

In another alternative of a composition comprising an additional therapeutic agent, the additional therapeutic agent is an anticholinergic drug as described above.

In another alternative of a composition comprising an additional therapeutic agent, the additional therapeutic agent is a xanthine compound as described above.

In another alternative of a composition comprising an additional therapeutic agent, the additional therapeutic agent is an anti-IgE antibody as described above.

In another alternative of a composition comprising an additional therapeutic agent, the additional therapeutic agent is a leukotriene antagonist as described above.

In another alternative of a composition comprising an additional therapeutic agent, the additional therapeutic agent is a phosphodiesterase IV inhibitor as described above.

In another alternative of a composition comprising an additional therapeutic agent, the additional therapeutic agent is a 5-lipoxygenase inhibitor as described above.

In another alternative of a composition comprising an additional therapeutic agent, the additional therapeutic agent is a mast cell stabilizer as described above.

In another alternative of a composition comprising an additional therapeutic agent, the additional therapeutic agent is a biological as described above.

In another alternative of a composition comprising an additional therapeutic agent, the additional therapeutic agent is a MAPK inhibitor as described above.

In another alternative of a composition comprising an additional therapeutic agent, the additional therapeutic agent is an arrestin-2 inhibitor as described above. Typically, the arrestin-2 inhibitor is selected from the group consisting of: a protein fragment of arrestin-2; a compound of Formula (A-I); an omega-3 fatty acid; a CXCR2 inhibitor; a MyD88 inhibitor; a MD2 inhibitor; an inhibitor of a GRK, such as a nitric acid donor that donates nitric oxide or a related redox species; inositol hexaphosphate; barbadin; ML-339; an inhibitor of protein kinase A; and a phospholipase C inhibitor.

In another alternative of a composition comprising an additional therapeutic agent, the additional therapeutic agent is an agent for treating a SARS-CoV-2 infection as described above. Typically, the agent for treating a SARS-CoV-2 infection is selected from the group consisting of a monoclonal antibody and a small molecule as described above.

When the agent for treating a SARS-CoV-2 infection is a monoclonal antibody, the monoclonal antibody can be selected from the group consisting of: (i) monoclonal antibodies that block the infection of susceptible cells by the SARS-CoV-2 virus; and (ii) monoclonal antibodies that act by other mechanisms. Typically, the antigen of the SARS-CoV-2 virus to which the monoclonal antibody binds is an epitope of the spike protein. The monoclonal antibody can be selected from the group consisting of bebtelovimab, bamlanivimab, etesevimab, casirivimab, imdemivab, tixagevimab, cilgavimab, regdanvimab, sotrovimab, vilobelimab, sarilumab, lenzilumab, and levilimab. When the agent for treating a SARS-CoV-2 infection is a small molecule, the small molecule can be selected from the group consisting of a fixed-dose combination of nirmatrelvir and ritonavir; a combination of nirmaltrevir and a CYP3A4 inhibitor selected from the group consisting of boceprevir, indinavir, nelfinavir, saquinavir, clarithromycin, telithromycin, ceritinib, mibefradil, nefazodone, ribociclib, tucatinib, chloramphenicol, ketoconazole, itraconazole, posaconazole, voriconazole, and cobicistat; remdesivir; favipravir; molnupravir; T-1105 (2-oxo-1H-pyrazine-3-carboxamide); T-1106 (4-[(2R,3R,4S,5R)-3,4-dihydroxy-5-(hydroxymethyl)oxolan-2-yl]-3-oxopyrazine-2-carboxamide); lufotrelvir; merafloxacin; olgotrevir; ASC-09 ([(3aS,4R,6aR)-2,3,3a,4,5,6a-hexahydrofuro[2,3-b]furan-4-yl] N-[(2S,3R)-4-[[2-[(1-cyclopentylpiperidin-4-yl)amino]-1,3-benzothiazol-6-yl]sulfonyl-(2-methylpropyl)amino]-3-hydroxy-1-phenylbutan-2-yl]carbamate); danoprevir; deuremidevir; disulfiram; ensitrelvir; simnotrelvir; talfirastide; and tipiracil. Alternatively, the small molecule can be: apabetalone; an inhibitor of TMPRSS2 that is typically selected from the group consisting of camostat, nafamostat, bromhexine, and N-0385 ((2S)—N-[(2S)-1-[[(2S)-1-(1,3-benzothiazol-2-yl)-5-(diaminomethylideneamino)-1-oxopentan-2-yl]amino]-1-oxo-3-phenylpropan-2-yl]-2-(methanesulfonamido)pentanediamide); an agent that blocks the activity of human protein eEF1A that is typically plitidepsin; a selective serotonin reuptake inhibitor that is typically selected from the group consisting of fluoxetine and fluvoxamine; an anti-mitotic agent that is typically selected from the group cabazitaxel, docetaxel, epothilone, ixabepilone, larotaxel, neoxaline, ortataxel, paclitaxel, sabizabulin, tesetaxel, demecolcine, vinblastine, nocodazole, vincristine, vindesine, vinflunine, and vinorelbine; an agent that is a SGLT2 inhibitor that is typically selected from the group consisting of canagliflozin, dapagliflozin, empagliflozin, ertugliflozin, ipragliflozin, luseogliflozin, remogliflozin etabonate, sergliflozin etabonate, sotagliflozin, and tofogliflozin; an IL-6 inhibitor that is typically selected from the group consisting of sarilumab and siltuximab; an IL-1 inhibitor that is typically selected from the group consisting of anakinra and canakinumab; a JAK pathway inhibitor that is typically tofacitinib; a TNF-α inhibitor that is typically infliximab; an IL-17 inhibitor that is typically secukinumab; an agent that inhibits MUC5AC that is typically selected from the group consisting of niclosamide and carbocisteine; an EGFR tyrosine kinase inhibitor that is typically selected from the group consisting of gefitinib, afatinib, brigatinib, erlotinib, lapatinib, neratinib, olmutinib, osimertinib, rociletinib, and vandetanib; a p38 mitogen-activated protein kinase inhibitor that is typically acumapimod; an anti-IDO antibody, clone 10.1; a statin that is typically selected from the group consisting of simvastatin, atorvastatin, fluvastatin, lovastatin, pitavastatin, pravastatin, and rosuvastatin; and BIO-11006 acetate.

In a composition according to the present invention as described above, the pharmaceutically acceptable carrier is typically selected from the group consisting of a solvent, a dispersion medium, a coating, an antibacterial agent, an antifungal agent, an isotonic agent, an absorption delaying agent, a preservative, a sweetening agent for oral administration, a thickening agent, a buffer, a liquid carrier, a wetting, solubilizing, or emulsifying agent; an acidifying agent, an antioxidant, an alkalinizing agent, a carrying agent, a chelating agent, a colorant, a complexing agent, a suspending or viscosity-increasing agent, a flavor or perfume, an oil, a penetration enhancer, a polymer, a stiffening agent, a protein, a carbohydrate, a bulking agent, and a lubricating agent.

In a composition according to the present invention as described above, the nadolol or derivative or analogue of nadolol is typically nadolol.

### BRIEF DESCRIPTION OF THE DRAWINGS

These and other features, aspects, and advantages of the present invention will become better understood with reference to the following description, appended claims, and accompanying drawings where:

FIG. 1 is a diagram of a blister pack holding dosage forms of inverse agonists or combinations of inverse agonists with other therapeutic agents for the treatment of infection with SARS-CoV-19 virus or sequelae of such infection or according to the present invention.

FIG. 2A is a graph showing that methacholine provocation significantly enhances airway resistance (R_{aw}) in a mouse model of asthma, relevant to respiratory symptoms caused by infection with SARS-CoV-2 virus or its sequelae.

FIG. 2B is a similar graph showing that saline provocation, as a control, does not significantly enhance airway resistance in a mouse model of asthma, relevant to respiratory symptoms caused by infection with SARS-CoV-2 virus or its sequelae.

FIG. 2C is a similar graph showing that the administration of a single intravenous bolus of salbutamol to asthmatic mice reduced the level of airway responsiveness to methacholine provocation and the level of airway resistance, relevant to respiratory symptoms caused by infection with SARS-CoV-2 virus or its sequelae.

FIG. 2D is a similar graph showing that no protection was observed when salbutamol was delivered to the mice for 28 days before methacholine provocation, relevant to respiratory symptoms caused by infection with SARS-CoV-2 virus or its sequelae.

FIG. 2E is a similar graph showing that when asthmatic mice were given a single intravenous bolus of alprenolol, a β-adrenergic antagonist with partial agonist activity, their airway responsiveness was diminished, relevant to respiratory symptoms caused by infection with SARS-CoV-2 virus or its sequelae.

FIG. 2F is a similar graph showing that when asthmatic mice were exposed to alprenolol for 28 days, their average methacholine dose-response relationship was similar to that obtained in nontreated mice, demonstrating that this drug provides no benefit upon chronic administration, relevant to respiratory symptoms caused by infection with SARS-CoV-2 virus or its sequelae.

FIG. 2G is a similar graph showing that a single intravenous bolus of carvedilol enhanced the airway responsiveness in the mouse model of asthma, relevant to respiratory symptoms caused by infection with SARS-CoV-2 virus or its sequelae.

FIG. 2H is a similar graph showing that chronic administration of carvedilol reduced the airway responsiveness of asthmatic mice to methacholine provocation, relevant to respiratory symptoms caused by infection with SARS-CoV-2 virus or its sequelae.

FIG. 2I is a similar graph showing that a single intravenous bolus of nadolol also enhanced the airway responsiveness of asthmatic mice similar to the effect observed for carvedilol, relevant to respiratory symptoms caused by infection with SARS-CoV-2 virus or its sequelae.

FIG. 2J is a similar graph showing that chronic administration of nadolol reduced the airway responsiveness of asthmatic mice to methacholine provocation similar to the effect observed for carvedilol on chronic administration of that drug, relevant to respiratory symptoms caused by infection with SARS-CoV-2 virus or its sequelae.

FIG. 3 is a graph showing the effects of administration of β-adrenergic receptor ligands on the peak airway responsiveness to cholinergic stimulation: ((A), after treatment with the β-adrenergic agonist salbutamol; (B) after acute treatments with β-adrenergic receptor inverse agonists; and (C) after chronic treatments with β-adrenergic receptor inverse agonists); these results are relevant to respiratory symptoms caused by infection with SARS-CoV-2 virus or its sequelae.

FIG. 4A is a graph showing the effect of combination therapy with carvedilol and salbutamol on airway hyperresponsiveness in asthmatic mice challenged with methacholine, relevant to respiratory symptoms caused by infection with SARS-CoV-2 virus or its sequelae.

FIG. 4B is a summary graph showing the results presented in FIG. 4A.

FIG. 5 is a graph showing the effect of acute combination therapy with nadolol and aminophylline on airway hyperresponsiveness in asthmatic mice challenged with methacholine, relevant to respiratory symptoms caused by infection with SARS-CoV-2 virus or its sequelae.

FIG. 6 is a graph showing the ratio of phospholipase C to actin in mice treated with various treatments, including long-term nadolol administration, to show that long-term nadolol administration decreases the activity of phospholipase C, relevant to respiratory symptoms caused by infection with SARS-CoV-2 virus or its sequelae.

FIG. 7A is a graph showing the effects of salbutamol administration upon airway hyperresponsiveness, relevant to respiratory symptoms caused by infection with SARS-CoV-2 virus or its sequelae.

FIG. 7B is a graph showing the effects of high-dose alprenolol administration upon airway hyperresponsiveness, relevant to respiratory symptoms caused by infection with SARS-CoV-2 virus or its sequelae.

FIG. 7C is a graph showing the effects of low-dose alprenolol administration upon airway hyperresponsiveness, relevant to respiratory symptoms caused by infection with SARS-CoV-2 virus or its sequelae.

FIG. 7D is a graph showing the effects of high-dose carvedilol administration upon airway hyperresponsiveness, relevant to respiratory symptoms caused by infection with SARS-CoV-2 virus or its sequelae.

FIG. 7E is a graph showing the effects of low-dose carvedilol administration upon airway hyperresponsiveness, relevant to respiratory symptoms caused by infection with SARS-CoV-2 virus or its sequelae.

FIG. 7F is a graph showing the effects of high-dose nadolol administration upon airway hyperresponsiveness, relevant to respiratory symptoms caused by infection with SARS-CoV-2 virus or its sequelae.

FIG. 7G is a graph showing the effects of low-dose nadolol administration upon airway hyperresponsiveness, relevant to respiratory symptoms caused by infection with SARS-CoV-2 virus or its sequelae.

FIG. 8 is a set of graphs showing the effects of long-term dosage of metoprolol and timolol upon airway hyperresponsiveness in asthmatic mice: (A) experimental results with metoprolol and timolol; (B) historical controls with non-challenged mice (Ctrl) and with challenged mice with no treatment (NTX); these results are relevant to respiratory symptoms caused by infection with SARS-CoV-2 virus or its sequelae.

FIG. 9 is a photomicrograph showing the occurrence of a mucus plug in the bronchus of an 8-year-old girl with fatal asthma, relevant to respiratory symptoms caused by infection with SARS-CoV-2 virus or its sequelae, which can be fatal.

FIG. 10 is a series of photomicrographs showing that nadolol is effective in preventing mucous metaplasia while the antagonist alprenolol is ineffective in preventing mucous metaplasia: top left, control; top right, sensitized/challenged mice without treatment showing mucous metaplasia; bottom left, sensitized/challenged mice after treatment with alprenolol showing no improvement in mucous metaplasia; bottom right, sensitized/challenged mice after treatment with nadolol showing nearly complete elimination of mucous metaplasia, relevant to respiratory symptoms caused by infection with SARS-CoV-2 virus or its sequelae.

FIG. 11 is a schematic diagram showing the mechanism of action of nadolol as contrasted with the mechanism of action of long-acting β-adrenoceptor agonists (“LABA”) and that nadolol (“INV102”) reverses epithelial changes via inhibition of the β-arrestin pathway in β₂ airway receptors.

FIG. 12 is a graph showing the effect of nadolol on the level of mucin 5AC in smokers treated with nadolol versus the results with a placebo; these results are relevant to respiratory symptoms caused by infection with SARS-CoV-2 virus or its sequelae.

FIG. 13 is a graph showing that nadolol blocks the β-arrestin pathway, as compared with carvedilol, propranolol, and alprenolol, which is relevant to the treatment of respiratory symptoms caused by infection with SARS-CoV-2 virus or its sequelae.

FIG. 14 is a set of photomicrographs showing the respiratory epithelium in: normal subject without airway disease (upper left); severe asthma (upper right); chronic bronchitis (lower left); and cystic fibrosis (lower right); this is relevant to treatment of respiratory symptoms caused by infection with SARS-CoV-2 virus or its sequelae.

### DETAILED DESCRIPTION OF THE INVENTION

### Definitions

Although any methods and materials similar to or equivalent to those described herein can be used in the practice or testing of embodiments described herein or other embodiments within the scope of the invention, some preferred methods, compositions, materials, and devices are described herein. However, in this context, it must be understood that this invention is not limited to the particular molecules, compositions, methodologies, or protocols described herein, as these aspects of the invention may vary in accordance with routine experimentation and optimization as is generally known in the art. It is also to be understood that the terminology used in the description and the claims is for the purpose of describing the particular versions or embodiments only, and is not intended to limit the scope of the embodiments as described herein as understood by one of skill in the art. Unless otherwise defined, all technical and scientific terms used herein have the same meanings as commonly understood by one of ordinary skill in the art to which this invention belongs. However, in case of any conflict of meanings, the present specification and claims, including definitions therein, shall control. Accordingly, in the context of the embodiments described herein, the following definitions apply.

As used herein and in the appended claims, the singular forms “a,” “an,” and “the” include references to the plural unless the context clearly dictates otherwise. Thus, for example, a reference to “a β₂-selective adrenergic agonist” is a reference to one or more β₂-selective adrenergic agonists or equivalents thereof known to those skilled in the art.

As used herein, the terms “comprise,” “include,” and linguistic variations thereof denote the presence of recited features, elements, method steps, or other components of the invention without the exclusion of the presence of additional/recited features, elements, method steps, or other components. Conversely, the terms “consisting of” and linguistic variations thereof denote the presence of recited features, elements, method steps, or other components of the invention and exclude any unrecited recited features, elements, method steps, or other components of the invention except for ordinarily-associated impurities. The phrase “consisting essentially of” and linguistic variations thereof denote the presence of recited features, elements, method steps, or other components of the invention and any additional features, elements, method steps, or other components of the invention that do not materially affect the basic nature of the composition, system, or method. Many embodiments herein are described using open “comprising” language; such embodiments also encompass embodiments described in terms of “consisting essentially of” or “consisting of” language, which may be alternatively claimed or described using such language, unless the context clearly excludes “consisting essentially of” or “consisting of” language. The terms “comprising,” “consisting essentially of,” and “consisting of” as used herein are applicable to both composition claims and method claims.

All chemical names used herein, including names of substituents, should be interpreted in light of the chemical nomenclature conventions of IUPAC and/or a modified format in which functional groups within a substituent are read in the order in which they branch from the scaffold or main structure. For example, in the modified nomenclature, methylsulfonylpropanol refers to CH₂SO₂CH₂CH₂CH₂OH or

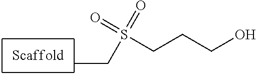

As another example, according to the modified nomenclature, a methylamine substituent is

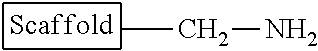

while an aminomethyl substituent is

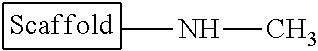

As used herein, the term “treating” means affecting a subject, tissue, organ, or cell to obtain a desired pharmacological and/or physiological effect, and can include inhibiting a condition, i.e., slowing or arresting its development, or relieving or ameliorating the effects of the condition, such as by causing reversal or regression of the effects of the condition. Such conditions can include, but are not necessarily limited to, respiratory conditions such as difficulty in breathing, abnormally low oxygen saturation, abnormal mucus hypersecretion, or other respiratory conditions associated with the sequelae of infection with SARS-CoV-2 virus.

As used herein, the term “preventing” means preventing a condition from occurring in a subject, tissue, organ, or cell that may be at risk of having the condition, but does not necessarily mean that the condition will not eventually develop, or that a subject, tissue, organ, or cell will not eventually develop a condition. Preventing includes delaying the onset of a condition in a subject, tissue, organ, or cell. Such conditions can include, but are not necessarily limited to, respiratory conditions such as difficulty in breathing, abnormally low oxygen saturation, abnormal mucus hypersecretion, or other respiratory conditions associated with the sequelae of infection with SARS-CoV-2 virus.

As used herein, the term “subject” broadly refers to any animal, including, but not limited to, humans and non-human mammals. The reference to non-human mammals includes, but is not limited to, socially or economically important animals or animals used for research including cattle, sheep, goats, horses, donkeys, pigs, llamas, alpacas, dogs, cats, rabbits, guinea pigs, rats, hamsters, gerbils, or mice. The reference to non-human mammals can also include captive wild animals such as foxes or deer and can also include non-human primates. Typically, the mammal is a human or a non-human primate. More typically, the mammal is a human. Unless specified, methods and compositions according to the present invention are not limited to treatment of humans. In general, when treatment of humans is intended, the term “patient” can be used in place of “subject.”

As used herein, the terms “effective amount,” “therapeutically effective amount,” or other equivalent terminology refer to the amount of a compound or compounds or to the amount of a composition sufficient to effect beneficial or desired results. The beneficial or desired results are typically a reduction in severity, symptoms, or duration of a disease or condition being treated and can generally be characterized as an amount of a therapeutic agent or composition effective to treat, ameliorate, or prevent a desired disease or condition, or to exhibit a detectable therapeutic or preventative effect. The use of such terminology cannot, unless specifically indicated, be interpreted as implying a complete cure for any disease or condition as recited herein. An effective amount can be administered in one or more administrations, applications, or dosages, and is not intended to be limited to a particular formulation or administration route unless a particular formulation or administration route is specified. The effect induced by the administration of a therapeutically effective amount can be detected by, for example, chemical markers, antigen levels, or changes in pathological indicators such as conventional indicators for lung function, including, but not limited to, tissue oxygen saturation, or other markers for other diseases or conditions. Therapeutic effects also can include subjective improvements in well-being, reduction of fatigue, reduction of pain, or increased energy noted by the subjects or their caregivers. The precise therapeutically effective amount for a subject will depend upon the subject's size, weight, and health, the nature and extent of the condition affecting the subject, the administration of other therapeutic agents administered to treat the particular disease or condition being treated or other diseases or conditions affecting the subject, as well as variables such as liver and kidney function that affect the pharmacokinetics of administered therapeutics. Thus, it is not useful to specify an exact effective amount in advance. However, the therapeutically effective amount for a given situation can be determined by routine experimentation and is within the judgment of the clinician.

As used herein, the terms “administration,” “administering,” or other equivalent terminology, refer to the act of giving a drug, prodrug, pharmaceutical composition, or other agent intended to provide therapeutic treatment to a subject or in vivo, in vitro, or ex vivo to cells, tissues, or organs. Exemplary routes of administration to the human body can be through space under the arachnoid membrane of the brain or spinal cord (intrathecal), the eyes (ophthalmic), mouth (oral), skin (topical or transdermal), nose (nasal), lungs or other portions of the respiratory tract (inhalant), oral mucosa (buccal), ear, rectal, vaginal, by injection (such as, but not limited to, intravenously, subcutaneously, intraperitoneally, or by other injection routes as known in the art). Suitable routes of administration will depend on the particular formulation being administered, the quantity of nadolol being administered, and the particular excipients in the formulation. Suitable routes of administration for therapeutic agents and pharmaceutical compositions described herein are as described below, particularly with respect to nadolol or with respect to derivatives or analogues of nadolol.

As used herein, the terms “co-administration” and “co-administering” refer to the administration of at least two agents, such as, for example, nadolol and a corticosteroid, or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful agent or agent, and/or when co-administration of two or more agents results in sensitization of a subject to beneficial effects of one of the agents via co-administration of the other agent. As used herein, the term “concurrent administration” refers to the administration of two or more active agents sufficiently close in time to achieve a combined therapeutic effect that is preferably greater than that which would be achieved by the administration of either agent alone. However, the occurrence of a combined therapeutic effect that is preferably greater than that which would be achieved by the administration of either agent alone is not a necessary requirement for concurrent administration.

As used herein, the term “pharmaceutical composition” refers to the combination of one or more therapeutically active agents with at least one carrier or excipient, inert or active, making the composition especially suitable for diagnostic or therapeutic use in vitro, in vivo or ex vivo. Typically, in the context of the present invention, the therapeutically active agent is nadolol or a derivative or analogue of nadolol as described herein. As detailed below, other agents can also be included.

As used herein, the terms “pharmaceutically acceptable” or “pharmacologically acceptable,” as used herein, refer to compositions, or components within compositions, that do not substantially produce adverse reactions, such as, but not limited to, toxic, allergic, or unwanted immunological reactions, when administered to a subject and also that do not substantially interact in a deleterious manner with any of the other components on the composition in which it is included.

As used herein, the term “pharmaceutically acceptable carrier” refers to any of the standard pharmaceutical carriers including, but not limited to, phosphate buffered saline solution, water, emulsions, such as oil/water or water/oil emulsions), and various types of wetting agents, any and all solvents, dispersion media, coatings, sodium lauryl sulfate, isotonic and absorption delaying agents, disintegrants such as potato starch or sodium starch glycolate), and the like. The carriers also can include stabilizers and preservatives. Suitable pharmaceutically acceptable carriers are described below. As used herein, the term “carrier” can include any and all solvents, dispersion media, vehicles, coatings, diluents, bulking agents, carrier solutions, suspensions, colloids, and forming and binding agents, any or all of which may include other pharmaceutical excipients as generally known in the art, including lubricants, antibacterial and antifungal agents, isotonic or absorption-delaying agents, buffers, antioxidants, other stabilizers including physical stabilizers such as thickeners or viscosity enhancers, coloring agents, flavoring or sweetening agents, and the like. The use of such media and agents with therapeutically active agents is well known in the art. Except insofar as any conventional medium or agent is incompatible with any therapeutically active agent or with any other medium or agent whose use is contemplated, its use in pharmaceutical compositions as described herein is contemplated.

As used herein, the term “pharmaceutically acceptable salt” refers to any pharmaceutically acceptable salt (e.g., acid or base) of a compound that is used in a method of the present invention or is a component of a pharmaceutical composition of the present invention, which, upon administration to a subject, is capable of providing a therapeutically active compound as described in the present application, including nadolol or another therapeutically active compound as described below, or an active metabolite or residue thereof. As is known to those of skill in the art, salts of the therapeutically active compounds described herein may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and other acids known in the art as suitable for formation of pharmaceutically acceptable salts. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metals (such as sodium or potassium) hydroxides, alkaline earth metals (such as calcium or magnesium), hydroxides, ammonia, and compounds of formula NW₄⁺, wherein W is C₁-C₄ alkyl, and the like. Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺, wherein W is a C₁-C₄ alkyl group), and the like. For therapeutic use, salts of the compounds herein are contemplated as being pharmaceutically acceptable. However, salts of acids and bases that are non-pharmaceutically acceptable may also find use, for example, in the preparation or purification of a pharmaceutically acceptable compound.

As used herein, the term “administration” or equivalent terminology refers to dispensing, applying, or tendering one or more agents to a subject. Administration can be performed using any of a number of methods known in the art, for example, administration via infusion (intravenous administration (i.v.)) or parenteral administration. By “parenteral administration” is typically meant intravenous, subcutaneous, or intramuscular administration, although other routes of administration are also possible.

As used herein, the term “instructions for administering a compound to a subject,” and grammatical equivalents thereof, includes instructions for using the compositions contained in a kit for the treatment of diseases or conditions such as those described herein. Such instructions, for example, provide dosing, routes of administration, or decision trees for treating physicians for correlating patient-specific characteristics with therapeutic courses of action. Such instructions may be part of a kit according to the present invention.

The following applies to analogues and derivatives of the compounds described in further detail below, including therapeutically active agents described herein for use with nadolol or a derivative or analogue of nadolol to treat sequelae of infection with SARS-CoV-2 virus as described herein. As used herein, “analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or hydrophobic or it may have altered reactivity as compared to the parent compound. The analogue may mimic the chemical and/or biological activity of the parent compound (i.e., it may have similar or identical activity), or, in some cases, may have increased or decreased activity. The analogue may be a naturally or non-naturally occurring variant of the original compound. Other types of analogues include isomers (enantiomers, diastereomers, and the like) and other types of chiral variants of a compound, as well as structural isomers. As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have different chemical or physical properties than the parent compound. For example, the derivative may be more hydrophilic or hydrophobic or it may have altered reactivity as compared to the parent compound. Derivatization (i.e., modification) may involve substitution of one or more moieties within the molecule (e.g., a change in functional group). The term “derivative” also includes conjugates and prodrugs of a parent compound (i.e., chemically modified derivatives which can be converted into the original compound under physiological conditions).

As used herein, the term “alkyl” refers to an unbranched, branched, or cyclic saturated hydrocarbyl residue, or a combination thereof, of from 1 to 12 carbon atoms, or in some cases up to 50 or more carbon atoms, that can be optionally substituted; the alkyl residues contain only C and H when unsubstituted. Typically, the unbranched or branched saturated hydrocarbyl residue is from 1 to 6 carbon atoms, preferably 1 to 4 carbon atoms, more preferably 1 to 3 carbon atoms, which is referred to herein as “lower alkyl.” When the alkyl residue is cyclic and includes a ring, it is understood that the hydrocarbyl residue includes at least three carbon atoms, which is the minimum number to form a ring. An alkyl group can be linear, branched, cyclic, or a combination thereof, and may contain from 1 to 50 or more carbon atoms, such as a straight chain or branched C₁-C₂₀ alkane. Examples of alkyl groups include but are not limited to methyl, ethyl, propyl, isopropyl, cyclopropyl, butyl isomers (e.g. n-butyl, isobutyl, and tert-butyl), cyclobutyl isomers (e.g. cyclobutyl, methylcyclopropyl, or other isomers), pentyl isomers, cyclopentane isomers, hexyl isomers, cyclohexane isomers, and the like. Unless specified otherwise (e.g., substituted alkyl group, heteroalkyl, alkoxy group, haloalkyl, alkylamine, thioalkyl, or other possible groups), an alkyl group contains carbon and hydrogen atoms only. As used herein, the term “linear alkyl” refers to a chain of carbon and hydrogen atoms (e.g., ethane, propane, butane, pentane, hexane, or other examples). A linear alkyl group may be referred to by the designation —(CH₂)_{q}CH₃, where q is 0-49. The designation “C₁-C₁₂ alkyl” or a similar designation refers to alkyl having from 1 to 12 carbon atoms such as methyl, ethyl, propyl isomers (e.g. n-propyl or isopropyl), butyl isomers, cyclobutyl isomers (e.g. cyclobutyl or methylcyclopropyl), pentyl isomers, cyclopentyl isomers, hexyl isomers, cyclohexyl isomers, heptyl isomers, cycloheptyl isomers, octyl isomers, cyclooctyl isomers, nonyl isomers, cyclononyl isomers, decyl isomers, cyclodecyl isomers, or other alternatives known in the art. Similar designations refer to alkyl with a number of carbon atoms in a different range. As used herein, the term “Cₓ-C_{y}” when used in conjunction with a chemical moiety, such as alkyl, alkenyl, alkynyl, or carbocycle is meant to include groups that contain from x to y carbons in the chain or ring. For example, the term “Cₓ-C_{y} alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, or other alternatives. The terms “Cₓ-C_{y} alkenyl” and “Cₓ-C_{y} alkynyl” refer to substituted or unsubstituted unsaturated aliphatic groups analogous in length and possible substitution to the alkyls described above, but that contain at least one double or triple bond respectively. The term “Cₓ-C_{y} carbocycle” refers to a substituted or unsubstituted carbocycle, that contain from x to y ring carbons. As used herein, the term “branched alkyl” refers to a chain of carbon and hydrogen atoms, without double or triple bonds, that contains a fork, branch, and/or split in the chain (e.g., 3,5-dimethyl-2-ethylhexane, 2-methyl-pentane, 1-methyl-cyclobutane, ortho-diethyl-cyclohexane, or other alternatives). “Branching” refers to the divergence of a carbon chain, whereas “substitution” refers to the presence of non-carbon/non-hydrogen atoms in a moiety. Unless specified otherwise (e.g., substituted branched alkyl group, branched heteroalkyl, branched alkoxy group, branched haloalkyl, branched alkylamine, branched thioalkyl, or other alternatives), a branched alkyl group contains carbon and hydrogen atoms only.

As used herein, the term “carbocycle,” “carbocyclyl,” or “carbocyclic” refers to a cyclic ring containing only carbon atoms in the ring, whereas the term “heterocycle” or “heterocyclic” refers to a ring comprising a heteroatom. The carbocycle can be fully saturated or partially saturated, but non-aromatic. For example, the general term “carbocyclyl” encompasses cycloalkyl. The carbocyclic and heterocyclic structures encompass compounds having monocyclic, bicyclic or multiple (polycyclic) ring systems; and such systems may mix aromatic, heterocyclic, and carbocyclic rings. Mixed ring systems are described according to the ring that is attached to the rest of the compound being described. Bicyclic or polycyclic rings may include fused or spiro rings. Carbocycles may include 3- to 10-membered monocyclic rings, 6- to 12-membered bicyclic rings, and 6- to 12-membered bridged rings. Each ring of a bicyclic or polycyclic carbocycle may be selected from saturated, unsaturated, and aromatic rings. In an exemplary embodiment, an aromatic carbocycle, e.g., phenyl, may be fused to a saturated or unsaturated ring, e.g., cyclohexane, cyclopentane, or cyclohexene. In some embodiments, the carbocycle is an aromatic carbocycle. In some embodiments, the carbocycle is a cycloalkyl. In some embodiments, the carbocycle is a cycloalkenyl. Exemplary carbocycles include cyclopentyl, cyclohexyl, cyclohexenyl, adamantyl, phenyl, indanyl, and naphthyl. An alkenyl group can be optionally substituted by one or more substituents such as those substituents described herein. A “non-aromatic carbocycle” includes rings and ring systems that are saturated, unsaturated, substituted or unsubstituted, but not aromatic or aryl rings or ring systems.

As used herein, the term “cycloalkyl” refers to a completely saturated mono- or multi-cyclic hydrocarbon ring system. When composed of two or more rings, the rings may be joined together in a fused, bridged or spiro-connected fashion. Cycloalkyl groups of the present application may range from three to ten carbons (C₃ to C₁₀). A cycloalkyl group may be unsubstituted, substituted, branched, and/or unbranched. Typical cycloalkyl groups include, but are not limited to, cyclopropyl, cyclobutyl, cyclopentyl, cyclohexyl, and the like. If substituted, the substituent(s) may be an alkyl or can be selected from those indicated above with regard to substitution of an alkyl group unless otherwise indicated. While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker.

As used herein, the term “heteroalkyl” refers to an alkyl group, as defined herein, wherein one or more carbon atoms are independently replaced by one or more heteroatoms (e.g., oxygen, sulfur, nitrogen, phosphorus, selenium, silicon, or combinations thereof). The alkyl group containing the non-carbon substitution(s) may be a linear alkyl, branched alkyl, cycloalkyl (e.g., cycloheteroalkyl), or combinations thereof. Non-carbons may be at terminal locations (e.g., 2-hexanol) or integral to an alkyl group (e.g., diethyl ether). In general, the “hetero” terms refer to groups that typically contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. In some cases, more than three heteroatoms may be present. Unless stated otherwise specifically in the specification, the heteroalkyl group may be optionally substituted as described herein. Representative heteroalkyl groups include, but are not limited to —OCH₂OMe, —OCH₂CH₂OMe, or —OCH₂CH₂OCH₂CH₂NH₂. For reasons of chemical stability, it is also understood that, unless otherwise specified, such groups do not include more than two contiguous heteroatoms except where an oxo group is present on N or S as in a nitro or sulfonyl group.

As used herein, the term “heteroalkylene” refers to an alkyl radical as described above where one or more carbon atoms of the alkyl is replaced with a heteroatom, e.g., O, N or S, or another heteroatom as described above. “Heteroalkylene” or “heteroalkylene chain” refers to a straight or branched divalent heteroalkyl chain linking the rest of the molecule to a radical group. Unless stated otherwise specifically in the specification, the heteroalkylene group may be optionally substituted as described herein. Representative heteroalkylene groups include, but are not limited to —OCH₂CH₂O—, —OCH₂CH₂OCH₂CH₂O—, or —OCH₂CH₂OCH₂CH₂OCH₂CH₂O—.

As used herein, the term “optionally substituted” indicates that the particular group or groups referred to as optionally substituted may have no non-hydrogen substituents, or the group or groups may have one or more non-hydrogen substituents consistent with the chemistry and pharmacological activity of the resulting molecule and such that a stable compound is formed thereby, i.e., a compound that does not spontaneously undergo transformation such as by rearrangement, cyclization, elimination, hydrolysis, lactone or lactam formation, or other reaction. If not otherwise specified, the total number of such substituents that may be present is equal to the total number of hydrogen atoms present on the unsubstituted form of the group being described; fewer than the maximum number of such substituents may be present. Where an optional substituent is attached via a double bond, such as a carbonyl oxygen (C═O), the group takes up two available valences on the carbon atom to which the optional substituent is attached, so the total number of substituents that may be included is reduced according to the number of available valences. As used herein, the term “substituted,” whether used as part of “optionally substituted” or otherwise, when used to modify a specific group, moiety, or radical, means that one or more hydrogen atoms are, each, independently of each other, replaced with the same or different substituent or substituents. Substitution of a structure depicted herein may result in removal or moving of a double bond or other bond, as will be understood by one in the field. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds that do not significantly alter the pharmacological activity of the compound in the context of the present invention. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. The heteroatoms such as nitrogen may have hydrogen substituents and/or any permissible substituents of organic compounds described herein which satisfy the valences of the heteroatoms.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, or I). When an alkyl group is substituted with more than one halogen radical, each halogen may be independently selected, e.g., 1-chloro, 2-fluoroethane.

As used herein, the term “aryl” refers to a monocyclic or fused bicyclic moiety having the well-known characteristics of aromaticity; examples include phenyl and naphthyl, which can be optionally substituted. Additional examples of aromatic rings include furan, benzofuran, isobenzofuran, pyrrole, indole, isoindole, thiophene, benzothiophene, benzo(c)thiophene, imidazole, benzimidazole, purine, pyrazole, indazole, oxazole, benzooxazole, isoxazole, benzisoxazole, thiazole, benzothiazole, benzene, naphthalene, pyridine, quinolone, isoquinoline, pyrazine, quinoxaline, pyrimidine, quinazoline, pyridazine, cinnoline, phthalazine, triazine (e.g., 1,2,3-triazine; 1,2,4-triazine; 1,3,5-triazine), and thiadiazole. The term “aromatic carbocycle” refers to an aromatic ring without heteroatoms present within the ring structure, such as, but not limited to benzene or naphthalene. Other terms that can be used include “aromatic ring,” “aryl group,” or “aryl ring.”

As used herein, the term “heterocycle,” “heterocyclyl,” “heterocyclic ring” or “heterocyclic group” is intended to mean a stable 4-, 5-, 6-, or 7-membered monocyclic or 7-, 8-, 9-, 10-, 11-, 12-, 13-, or 14-membered bicyclic heterocyclic ring which is saturated, partially unsaturated, or fully unsaturated or aromatic, and which consists of carbon atoms and 1, 2, 3 or 4 heteroatoms independently selected from N, O, and S; and including any bicyclic group in which any of the above-defined heterocyclic rings is fused to a benzene ring. Other heteroatoms, such as P, Se, B, or Si, can be included in some alternatives. The nitrogen and sulfur heteroatoms may optionally be oxidized. The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. When the term “heterocycle,” “heterocyclyl,” “heterocyclic ring” or “heterocyclic group” is used, it is intended to include heteroaryl unless heteroaryl is excluded. Examples of heterocycles include, but are not limited to, acridinyl, azocinyl, benzimidazolyl, benzofuranyl, benzothiofuranyl, benzoxazolyl, benzoxazolinyl, benzthiazolyl, benztriazolyl, benztetrazolyl, benzisoxazolyl, benzisothiazolyl, benzimidazolinyl, carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocycles.

As used herein, the term “non-aromatic heterocycle” refers to a cycloalkyl or cycloalkenyl, as defined herein, wherein one or more of the ring carbons are replaced by a moiety selected from —O—, —N═, —NR—, —C(O)—, —S—, —S(O)— or —S(O)₂—, wherein R is hydrogen, C₁-C₈ alkyl or a nitrogen protecting group, with the proviso that the ring of such a group does not contain two adjacent O or S atoms. In some alternatives, other heteroatoms including P, Se, B, or Si can be included. Non-limiting examples of non-aromatic heterocycles, as used herein, include morpholino, pyrrolidinyl, pyrrolidinyl-2-one, piperazinyl, piperidinyl, piperidinylone, 1,4-dioxa-8-aza-spiro(4.5)dec-8-yl, 2H-pyrrolyl, 2-pyrrolinyl, 3-pyrrolinyl, 1,3-dioxolanyl, 2-imidazolinyl, imidazolidinyl, 2-pyrazolinyl, pyrazolidinyl, 1,4-dioxanyl, 1,4-dithianyl, thiomorpholinyl, azepanyl, hexahydro-1,4-diazepinyl, tetrahydrofuranyl, dihydrofuranyl, tetrahydrothienyl, tetrahydropyranyl, dihydropyranyl, tetrahydrothiopyranyl, thioxanyl, azetidinyl, oxetanyl, thietanyl, oxepanyl, thiepanyl, 1,2,3,6-tetrahydropyridinyl, 2H-pyranyl, 4H-pyranyl, dioxanyl, 1,3-dioxolanyl, dithianyl, dithiolanyl, dihydropyranyl, dihydrothienyl, dihydrofuranyl, imidazolinyl, imidazolidinyl, 3-azabicyclo(3.1.0)hexanyl, and 3-azabicyclo(4.1.0)heptanyl, 3,8-diazabicyclo(3.2.1)octanyl, and 2,5-diazabicyclo(2.2.1)heptanyl. In certain embodiments, a non-aromatic heterocyclic ring is aziridine, thiirane, oxirane, oxaziridine, dioxirane, azetidine, oxetan, thietane, diazetidine, dioxetane, dithietane, pyrrolidine, tetrahydrofuran, thiolane, imidazolidine, pyrazolidine, oxazolidine, isoxazolidine, thiazolidine, isothiazolidine, dioxolane, dithiolane, piperdine, oxane, thiane, piperazine, morpholine, thiomorpholine, dioxane, dithiane, trioxane, thithiane, azepane, oxepane, thiepane, homopiperazine, or azocane.

As used herein, the terms “heteroaryl” or “heteroaromatic” refer to monocyclic, bicyclic, or polycyclic ring systems, wherein at least one ring in the system is aromatic and contains at least one heteroatom, for example, nitrogen, oxygen and sulfur. Each ring of the heteroaromatic ring systems may contain 3 to 7 ring atoms. Exemplary heteroaromatic monocyclic ring systems include 5- to 7-membered rings whose ring structures include one to four heteroatoms, for example, one or two heteroatoms. The inclusion of a heteroatom permits aromaticity in 5-membered rings as well as in 6-membered rings. Typical heteroaromatic systems include monocyclic C₅-C₆ heteroaromatic groups such as pyridyl, pyrimidyl, pyrazinyl, thienyl, furanyl, pyrrolyl, pyrazolyl, thiazolyl, oxazolyl, triazolyl, triazinyl, tetrazolyl, tetrazinyl, and imidazolyl, as well as the fused bicyclic moieties formed by fusing one of these monocyclic heteroaromatic groups with a phenyl ring or with any of the heteroaromatic monocyclic groups to form a C₅-C₁₀ bicyclic group such as indolyl, benzimidazolyl, indazolyl, benzotriazolyl, isoquinolyl, quinolyl, benzothiazolyl, benzofuranyl, pyrazolylpyridyl, quinazolinyl, quinoxalinyl, cinnolinyl, and other ring systems known in the art. Any monocyclic or fused ring bicyclic system that has the characteristics of aromaticity in terms of delocalized electron distribution throughout the ring system is included in this definition. This definition also includes bicyclic groups where at least the ring that is directly attached to the remainder of the molecule has the characteristics of aromaticity, including the delocalized electron distribution that is characteristic of aromaticity. Typically the ring systems contain 5 to 12 ring member atoms and up to four heteroatoms, wherein the heteroatoms are selected from the group consisting of N, O, and S. Frequently, the monocyclic heteroaryls contain 5 to 6 ring members and up to three heteroatoms selected from the group consisting of N, O, and S; frequently, the bicyclic heteroaryls contain 8 to 10 ring members and up to four heteroatoms selected from the group consisting of N, O, and S. The number and placement of heteroatoms in heteroaryl ring structures is in accordance with the well-known limitations of aromaticity and stability, where stability requires the heteroaromatic group to be stable enough to be exposed to water at physiological temperatures without rapid degradation. As used herein, the term “hydroxyheteroaryl” refers to a heteroaryl group including one or more hydroxyl groups as substituents; as further detailed below, further substituents can be optionally included. As used herein, the terms “haloaryl” and “haloheteroaryl” refer to aryl and heteroaryl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. As used herein, the terms “haloalkyl,” “haloalkenyl,” and “haloalkynyl” refer to alkyl, alkenyl, and alkynyl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. When a range of values is listed, such as for the number of carbon atoms in an alkyl group, it is intended to encompass each value and subrange within the range. For example, “C₁-C₆ alkyl” includes alkyl groups with 1, 2, 3, 4, 5, or 6 carbon atoms and all possible subranges.

As used herein, the term “hydroxyaryl” refers to an aryl group including one or more hydroxyl groups as substituents; as further detailed below, further substituents can be optionally included on the carbon atoms of the aryl group.

As used herein, the term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolyzable esters of the present compounds are the compounds whose carboxyls are present in the form of hydrolyzable ester groups. That is, these esters are pharmaceutically acceptable and can be hydrolyzed to the corresponding carboxy acid in vivo.

As used herein, the term “alkenyl” refers to an unbranched, branched or cyclic hydrocarbyl residue having one or more carbon-carbon double bonds. Typically, the hydrocarbyl residue has from 2 to 12 carbon atoms (C₂-C₁₂ alkenyl). In certain embodiments, an alkenyl comprises two to eight carbon atoms (C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. An alkenyl group can be optionally substituted by one or more substituents such as those substituents described herein. With respect to the use of “alkenyl,” the presence of multiple double bonds cannot product an aromatic ring structure.

As used herein, the term “alkynyl” refers to an unbranched, branched, or cyclic hydrocarbyl residue having one or more carbon-carbon triple bonds; the residue can also include one or more double bonds. Typically, the hydrocarbyl residue has from 2 to 12 carbon atoms (C₂-C₁₂ alkynyl). In certain embodiments, an alkenyl comprises two to eight carbon atoms (C₂-C₈ alkynyl). In certain embodiments, an alkenyl comprises two to six carbon atoms (i.e., C₂-C₆ alkynyl). In other embodiments, an alkenyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like. With respect to the use of “alkynyl,” the presence of multiple double bonds in addition to the one or more triple bonds cannot produce an aromatic ring structure.

As used herein, the term “alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₁₀ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises only one carbon atom (i.e., C₁ alkylene or a —CH₂ group). An alkylene group can be optionally substituted by one or more substituents such as those substituents described herein.

As used herein, the term “alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). An alkenylene group can be optionally substituted by one or more substituents such as those substituents described herein.

As used herein, “alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). An alkynylene group can be optionally substituted by one or more substituents such as those substituents described herein.

As used herein, the term “amine” or “amino” includes primary, secondary, and tertiary amines wherein each non-hydrogen group on nitrogen may be selected from alkyl, aryl, and the like. Amines include but are not limited to —NH₂, —NH-phenyl, —NH—CH₃, —NH—CH₂CH₃, and —N(CH₃)benzyl. The amino group can be optionally substituted. For example, the term can include NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “amide” or “amido” includes C- and N-amide groups, e.g., —C(O)NR₂, and —NRC(O)R groups, respectively, where R can be H, alkyl, aryl, or other groups, which can be optionally substituted. Amide groups therefore include but are not limited to —C(O)NH₂, —NHC(O)H, —C(O)NHCH₂CH₃, —NHC(O)CH₃, or —C(O)N(CH₂CH₃)phenyl.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

As used herein, similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

As used herein, the term “heteroatom” refers to any atom that is not carbon or hydrogen, such as nitrogen, oxygen or sulfur. When it is part of the backbone or skeleton of a chain or ring, a heteroatom must be at least divalent, and will typically be selected from N, O, P, and S, more typically from N, O, and P. The term “heteroatom” can include, in some contexts, other atoms, including selenium, silicon, or boron.

As used herein, the term “alkanoyl” refers to an alkyl group covalently linked to a carbonyl (C═O) group. The term “lower alkanoyl” refers to an alkanoyl group in which the alkyl portion of the alkanoyl group is C₁-C₆. The alkyl portion of the alkanoyl group can be optionally substituted as described above. The term “alkylcarbonyl” can alternatively be used. Similarly, the terms “alkenylcarbonyl” and “alkynylcarbonyl” refer to an alkenyl or alkynyl group, respectively, linked to a carbonyl group.

As used herein, the term “alkoxy” refers to an alkyl group covalently linked to an oxygen atom; the alkyl group can be considered as replacing the hydrogen atom of a hydroxyl group. The term “lower alkoxy” refers to an alkoxy group in which the alkyl portion of the alkoxy group is C₁-C₆. The alkyl portion of the alkoxy group can be optionally substituted as described above. As used herein, the term “haloalkoxy” refers to an alkoxy group in which the alkyl portion is substituted with one or more halo groups.

As used herein, the term “sulfo” refers to a sulfonic acid (—SO₃H) substituent.

As used herein, the term “sulfamoyl” refers to a substituent with the structure S(O₂)NH₂, wherein the nitrogen of the NH₂ portion of the group can be optionally substituted as described above.

As used herein, the term “carboxyl” refers to a group of the structure —C(O₂)H.

As used herein, the term “carbamyl” refers to a group of the structure —C(O₂)NH₂, wherein the nitrogen of the NH₂ portion of the group can be optionally substituted as described above.

As used herein, the terms “monoalkylaminoalkyl” and “dialkylaminoalkyl” refer to groups of the structure -Alk₁-NH-Alk₂ and -Alk₁-N(Alk₂)(Alk3), wherein Alk₁, Alk₂, and Alk₃ refer to alkyl groups as described above.

As used herein, the term “alkylsulfonyl” refers to a group of the structure —S(O)₂-Alk wherein Alk refers to an alkyl group as described above. The terms “alkenylsulfonyl” and “alkynylsulfonyl” refer analogously to sulfonyl groups covalently bound to alkenyl and alkynyl groups, respectively. The term “arylsulfonyl” refers to a group of the structure —S(O)₂—Ar wherein Ar refers to an aryl group as described above. The term “aryloxyalkylsulfonyl” refers to a group of the structure —S(O)₂-Alk-O—Ar, where Alk is an alkyl group as described above and Ar is an aryl group as described above. The term “arylalkylsulfonyl” refers to a group of the structure —S(O)₂-AlkAr, where Alk is an alkyl group as described above and Ar is an aryl group as described above.

As used herein, the term “alkyloxycarbonyl” refers to an ester substituent including an alkyl group wherein the carbonyl carbon is the point of attachment to the molecule. An example is ethoxycarbonyl, which is CH₃CH₂OC(O)—. Similarly, the terms “alkenyloxycarbonyl,” “alkynyloxycarbonyl,” and “cycloalkylcarbonyl” refer to similar ester substituents including an alkenyl group, alkenyl group, or cycloalkyl group respectively. Similarly, the term “aryloxycarbonyl” refers to an ester substituent including an aryl group wherein the carbonyl carbon is the point of attachment to the molecule. Similarly, the term “aryloxyalkylcarbonyl” refers to an ester substituent including an alkyl group wherein the alkyl group is itself substituted by an aryloxy group.

As used herein, the term “absent” when used in reference to a functional group or substituent, particularly in reference to the chemical structure of a compound, means that the particular functional group or substituent is not present in the compound being described. When used in reference to a substituent, the absence of the substituent typically means that the bond to the substituent is absent and that absence of the bond is compensated for with a H atom. When used in reference to a position within a chain or ring, the absence of the position typically means that the two positions otherwise connected by the absent position are instead directly connected by a covalent bond.

Other combinations of substituents are known in the art and, are described, for example, in U.S. Pat. No. 8,344,162 to Jung et al. or in PCT Patent Application Publication No. WO 2019/204768 by Kobilka et al. For example, the term “thiocarbonyl” and combinations of substituents including “thiocarbonyl” include a carbonyl group in which a double-bonded sulfur replaces the normal double-bonded oxygen in the group. The term “alkylidene” and similar terminology refer to an alkyl group, alkenyl group, alkynyl group, or cycloalkyl group, as specified, that has two hydrogen atoms removed from a single carbon atom so that the group is double-bonded to the remainder of the structure.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. The term “solvate” typically means a physical association of a compound involving varying degrees of ionic and/or covalent bonding, including hydrogen bonding. In certain instances the solvate will be capable of isolation, for example when one or more solvent atoms are incorporated into the crystal lattice of the crystalline solid. The term “solvate” encompasses both solution-phase and isolatable solvates. Suitable solvates in which the solvent is other than water include, but are not limited to, ethanolates or methanolates. When water is the solvent, the corresponding solvate is a “hydrate.” Examples of hydrates include, but are not limited to, hemihydrate, monohydrate, dihydrate, trihydrate, hexahydrate, and other hydrated forms. It should be understood by one of ordinary skill in the art that the pharmaceutically acceptable salt and/or prodrug of compounds described herein for use in methods or compositions according to the present invention may also exist in a solvate form. When the solvate is a hydrate, the hydrate is typically formed via hydration which is either part of the preparation of the present compound or through natural absorption of moisture by the anhydrous compound of the present invention. Additionally, compounds may exist as clathrates or other complexes, which are therapeutic agent-host inclusion complexes wherein the therapeutic agent and the host are present in stoichiometric or non-stoichiometric amounts.

Certain compounds described herein for use in methods and compositions according to the present invention possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present invention unless specific isomers are excluded. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. The term “tautomer,” as used herein, refers to one of two or more structural isomers that exist in equilibrium and that are readily converted from one form to another. Examples of tautomerism include, but are not limited to, keto-enol tautomerism, enamine-imine tautomerism, and lactam-lactim tautomerism. Unless one of the tautomeric alternatives is expressly excluded, all such tautomeric forms are intended to be within the scope of the invention.

Accordingly, in some alternatives, methods and compositions according to the present invention can encompass analogues and derivatives of small molecules that are optionally substituted, provided that the optionally substituted small molecules possess substantially equivalent pharmacological activity to the unsubstituted small molecules as defined in terms of their activity. The activity can be assayed by methods known in the art, including enzyme assays, assays to determine the effect of the analogue or derivative on respiratory function, or other methods known in the art. Such optionally substituted small molecules include, but are not necessarily limited to, molecules in which the substitutions are considered to be bioisosteric. Bioisosterism is a well-known tool for predicting the biological activity of compounds, based on the premise that compounds with similar size, shape, and electron density can have similar biological activity. To form a bioisostere of a given molecule, one can replace one or more atoms or groups in the original molecule with known bioisosteric replacements for that atom or group. Known bioisosteric replacements include, but are not necessarily limited to, the interchangeability of —F, —OH, —NH₂, —Cl, and —CH₃, the interchangeability of —Br and -i-C₃H₇; the interchangeability of —I and -t-C₄H₉; the interchangeability of —C—, —S—, —NH₂—, —CH₂—, and —Se—; the interchangeability of —N═, —CH═, and —P═ in cyclic or noncyclic moieties; the interchangeability of phenyl and pyridyl groups; the interchangeability of —C═C— and —S— (for example, benzene and thiophene); the interchangeability of an aromatic nitrogen (R₁—N(R₃)—R₂) for an unsaturated carbon ((R₁—C(═R₃)—R₂); and the interchangeability of —CO—, —SO—, and —SO₂—. Other alternatives for bioisosteric replacements are known in the art.

When a therapeutically active compound employed in methods or compositions according to the present application is a protein, protein fragment, polypeptide, or peptide, the protein, protein fragment, polypeptide, or peptide can be modified by the inclusion of one or more conservative amino acid substitutions, as long such conservative amino acid substitutions substantially preserve the biological activity of the therapeutically active compound. More specifically, in a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al., Molecular Biology of the Gene, 4th Edition, 1987, Benjamin/Cummings, p. 224). In particular, such a conservative variant has a modified amino acid sequence, such that the change or changes do not substantially alter the protein's (the conservative variant's) secondary or tertiary structure and/or activity, specifically binding activity in this context. Conservative amino acid substitution generally involves substitutions of amino acids with residues having similar properties (e.g., acidic, basic, positively or negatively charged, polar or non-polar, or other similarities) such that the substitutions of even critical amino acids do not substantially alter structure and/or activity. Conservative substitution tables providing functionally similar amino acids are well known in the art. For example, one exemplary guideline to select conservative substitutions includes (original residue followed by exemplary substitution): Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu. An alternative exemplary guideline uses the following six groups, each containing amino acids that are conservative substitutions for one another: (1) alanine (A or Ala), serine (S or Ser), threonine (T or Thr); (2) aspartic acid (D or Asp), glutamic acid (E or Glu); (3) asparagine (N or Asn), glutamine (Q or Gln); (4) arginine (R or Arg), lysine (K or Lys); (5) isoleucine (I or Ile), leucine (L or Leu), methionine (M or Met), valine (V or Val); and (6) phenylalanine (F or Phe), tyrosine (Y or Tyr), tryptophan (W or Trp); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. As another example, for some purposes, one may regard all non-polar amino acids as conservative substitutions for each other. In some alternatives, the amino acid substitution can be a D-amino acid or a non-canonical amino acid. Non-canonical amino acids are described in A. J. Link et al., “Non-Canonical Amino Acids in Protein Engineering,” Curr. Opin. Biotechnol. 14: 603-609 (2003).

As used herein, unless further defined or limited, the term “antibody” encompasses both polyclonal and monoclonal antibodies, as well as genetically engineered antibodies such as chimeric, humanized or fully human antibodies of the appropriate binding specificity. As used herein, unless further defined or limited, the term “antibody” also encompasses antibody fragments such as sFv, Fv, Fab, Fab′ and F(ab)′₂ fragments. In many cases, it is preferred to use monoclonal antibodies. In some contexts, antibodies can include fusion proteins comprising an antigen-binding site of an antibody, and any other modified immunoglobulin molecule comprising an antigen recognition site (i.e., antigen-binding site) as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins, therapeutic agents, antimetabolites, or radioisotopes; in some cases, conjugation occurs through a linker or through noncovalent interactions such as an avidin-biotin or streptavidin-biotin linkage.

In many cases, when antibodies that have been described and for which the amino acid sequence of both their heavy chains and light chains are completely known and are within the scope of the present invention, variants of such antibodies that have one or more conservative amino acid substitutions and substantially retain their binding affinity for the epitopes to which they specifically bind are also within the scope of the present invention. As used herein, the term “substantially retain their binding affinity” means that the variants that are within the scope of the invention retain at least 60% of the binding affinity for the epitope of the original antibody. Preferably, the variants retain at least 75% of the binding affinity for the epitope of the original antibody. More preferably, the variants retain at least 85% of the binding affinity for the epitope of the original antibody. Still more preferably, the variants retain at least 95% of the binding affinity for the epitope of the original antibody. Most preferably, variants retain at least 98% of the binding affinity for the epitope of the original antibody.

Therapeutically active compounds described herein, including many small-molecule therapeutically active compounds, can, in some alternatives, also be formulated and administered as a prodrug. As used herein, the term “prodrug” refers to compounds that are transformed in vivo to yield a disclosed compound or a pharmaceutically acceptable form of the compound. In some embodiments, a prodrug is a compound that may be converted under physiological conditions or by solvolysis to a biologically active compound as described herein. Thus, the term “prodrug” refers to a precursor of a biologically active compound that is pharmaceutically acceptable. A prodrug can be inactive when administered to a subject, but is then converted in vivo to an active compound, for example, by hydrolysis (e.g., hydrolysis in blood or a tissue). In certain cases, a prodrug has improved physical and/or delivery properties over a parent compound from which the prodrug has been derived. The prodrug often offers advantages of solubility, tissue compatibility, or delayed release in a mammalian organism (H. Bundgard, Design of Prodrugs (Elsevier, Amsterdam, 1988), pp. 7-9, 21-24), incorporated herein by this reference. A discussion of prodrugs is provided in T. Higuchi et al., “Pro-Drugs as Novel Delivery Systems,” ACS Symposium Series, Vol. 14 and in E. B. Roche, ed., Bioreversible Carriers in Drug Design (American Pharmaceutical Association & Pergamon Press, 1987). Exemplary advantages of a prodrug can include, but are not limited to, its physical properties, such as enhanced water solubility for parenteral administration at physiological pH compared to the parent compound, enhanced absorption from the digestive tract, or enhanced drug stability for long-term storage.

The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound in such a way that the modifications are cleaved, either in routine manipulation or in vivo, to yield the parent therapeutically active compound. Prodrugs include compounds wherein a hydroxy, amino, or mercapto group is covalently bonded to any group that, when the prodrug of the active compound is administered to a subject, cleaves to form a free hydroxy, free amino, or free mercapto group, respectively. Examples of prodrugs include, but are not limited to, formate or benzoate derivatives of an alcohol or acetamide, formamide or benzamide derivatives of a therapeutically active agent possessing an amine functional group available for reaction, and the like.

For example, if a therapeutically active agent or a pharmaceutically acceptable form of a therapeutically active agent contains a carboxylic acid functional group, a prodrug can comprise an ester formed by the replacement of the hydrogen atom of the carboxylic acid group with a group such as C₁₋₈ alkyl, C₂₋₁₂ alkanoyloxymethyl, 1-(alkanoyloxy)ethyl having from 4 to 9 carbon atoms, 1-methyl-1-(alkanoyloxy)ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, γ-butyrolacton-4-yl, di-N,N(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino-, or morpholino(C₂-C₃)alkyl.

Similarly, if a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1-((C₁-C₆))alkanoyloxy)ethyl, 1-methyl-1-((C₁-C₆)alkanoyloxy)ethyl (C₁-C₆)alkoxycarbonyloxymethyl, N(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, C(OH)C(O)OY¹ wherein Y¹ is H, (C₁-C₆)alkyl or benzyl, C(OY²)Y³ wherein Y² is (C₁-C₄) alkyl and Y³ is (C₁-C₆)alkyl, carboxy(C₁-C₆)alkyl, amino(C₁-C₄)alkyl or mono-N or di-N,N(C₁-C₆)alkylaminoalkyl, C(Y⁴)Y⁵ wherein Y⁴ is H or methyl and Y⁵ is mono-N or di-N,N(C₁-C₆)alkylamino, morpholino, piperidin-1-yl or pyrrolidin-1-yl.

The use of prodrug systems is described in T. Jarvinen et al., “Design and Pharmaceutical Applications of Prodrugs” in Drug Discovery Handbook (S. C. Gad, ed., Wiley-Interscience, Hoboken, NJ, 2005), ch. 17, pp. 733-796.

Pharmacology of Nadolol and its Use in Treating Sequelae of Infection with SARS-CoV-2 Virus

Airway mucus is an adhesive viscoelastic gel which, in healthy patients, is important in maintaining lung function and health. However, in patients with various diseases and conditions affecting the respiratory tract, including, but not limited to, infection with SARS-CoV-2 virus or the sequelae of such infection, pathological mucus hypersecretion may cause airway obstruction and lead to respiratory distress (W. Lu et al., “Elevated MUC1 and MUC5AC Mucin Protein Levels in Airway Mucus of Critically Ill COVID-19 Patients,” J. Med. Virol. 93: 582-584 (2021)). Mucin (MUC) glycoproteins are the major macromolecular components of mucus. These glycoproteins are classified into two major types: the gel-forming secreted mucins, such as MUC5AC, MUC2, and MUC5B, with MUC5AC being the most important, and the membrane-tethered mucins, such as MUC1, MUC4, and MUC16, with MUC1 being the most important. Compared to healthy controls, airway mucus from patients infected with SARS-CoV-2 virus had a higher level of MUC5AC, MUC1, and MUC1-CT fragment. MUC1 is a membrane-tethered mucin expressed on the apical surface of epithelial cells. Since the MUC1-CT fragment derived from MUC-1 is on the cytoplasmic side of epithelial cells, the elevated sputum concentrations of the MUC1-CT fragment in patients infected with SARS-CoV-2 virus could come from detached and disrupted epithelial cells, which is consistent with pathological findings of diffuse alveolar damage with fibromyxoid exudates and macrophage infiltration in the lung tissue of patients infected with SARS-CoV-2 virus. In fact, it has been recognized that the levels of MUC5AC, MUC5B, as well as the membrane mucins MUC4, MUC16, and MUC20, have all been elevated in the lung club cells of patients infected with SARS-CoV-2 virus.

Increased mucus production is increasingly recognized as an early sign in acute disease caused by SARS-CoV-2 virus (M. A. Khan et al., “Cytokine Storm and Mucus Hypersecretion in COVID-19: Review of Mechanisms,” Inflamm. Res. 14: 175-189 (2021)). In particular, nasal blockage and respiratory congestion are among the most common symptoms experienced by patients infected with SARS-CoV-2 virus. These symptoms can be particularly severe or even lethal due to the formation of mucus plugs. It has now been established that infection with SARS-CoV-2 virus causes an allergic reaction in respiratory tract mucosa, which activates mucin secretion and modulates its chemical structure to enable the virus to enter the cells. Thereafter, infection with the SARS-CoV-2 virus initiates neutrophil and mucus-mediated inflammatory pathways.

The increase in mucus production and secretion during the course of SARS-CoV-2 infection is likely due to mucus cell metaplasia. In the pulmonary system, mucus is a component of the endothelial lining fluid (ELF) or airway surface liquid (ASL). The major part of the respiratory tract is covered by mucus. The ASL comprises a sol layer and an overlying gel layer which are known as the periciliary liquid layer (PCL) and the mucus layer, respectively. Mucus consists of about 95% water. The major non-aqueous component is the protein mucin, while proteoglycans, lipids, proteins, and DNA are also present in smaller quantities. Mucin is secreted by goblet cells which are columnar epithelial cells present in the respiratory, gastrointestinal and reproductive tracts. Mucin-containing secretory vesicles are present at the upper surface of goblet cells. Mucin functions primarily as a barrier, and includes the protein components MUC5A and MUC5B. Chains of carbohydrates make up about 80% of the weight of mucins. The release of mucin is governed by fusion proteins such as SNARE (N-ethylmaleimide-sensitive factor attachment protein receptor) and MARCKS (myristoylated alanine-rich C kinase substrate); secretion occurs in an environment of high pH and low Ca²⁺ concentration.

It has been shown that the cytokines IL-4, IL-5, IL-9, and IL-13 upregulate mucin gene expression of mucus cell secretion. Other factors, including smoking, generate reactive oxygen species, which in turn upregulate numerous downstream cascades, which trigger multiple signaling pathways via mitogen-activated protein kinase and other signaling cascades. These signaling cascades induce goblet cell differentiation in the airway, causing excessive synthesis and secretion of mucin. This process occurs after infection with SARS-CoV-2 virus. This results in goblet cell differentiation and hyperplasia in the airway, leading to the synthesis of MUC proteins, especially MUC5AC, MUC5B, MUC1, MUC2, and MUC4, followed by their secretion. The SARS-CoV-2 virus can cause formation of mucus plugs in infected patients, causing airway obstruction and respiratory failure in a significant portion of patients infected with the SARS-CoV-2 virus. Severe mucoid tracheitis is detected in about one-third of autopsies of patients who died with infection with the SARS-CoV-2 virus.

Infection with the SARS-CoV-2 virus is believed to stimulate mucus hypersecretion through a number of pathways, including: (i) the JAK-STAT signaling pathway, activated directly or indirectly by IL-4, IL-8, IL-9, and IL-2, as well as IL-7, IL-10, IL-1β, IL-6, IL-13, and TNF-α; (ii) MAPK-mediated signaling, activated directly or indirectly by IL-6, IL-5, IL-2, IL-17, and IL-1β; (iii) NF-κB-mediated signaling, increasing the transcription of IL-1, IL-6, IL-8, and TNF-α; and (iv) leukotriene-mediated mucus oversecretion, particularly involving binding of leukotriene LTE4 to its receptor, cysteinyl leukotriene receptor 1 (CysLT1).

Additionally, it has been determined that several cytokines actually exert an anti-inflammatory role and can protect against inflammation and the occurrence of a cytokine storm following infection by the SARS-CoV-2 virus. These anti-inflammatory cytokines include IL-37, IL-27, IL-35, and IL-38.

The interleukin IL-37 is capable of inhibiting pro-inflammatory effects that are mediated through activation of receptors belonging to the interleukin-1 receptor/toll-like receptor (TIR) superfamily such as TIRs 2 and 4 and the IL-1 receptor. IL-37 has also been shown to modulate inflammation by downregulating the responses of Th₁, Th₂, and Th₁₇ cells. Local administration of IL-37 can reduce eosinophil levels in bronchoalveolar (BAL) fluid and respiratory tract tissues. Upon binding to IL-18R, IL-37 suppresses the expression of IL-la, IL-6, IL-1β, TNF-α, G-CSF, and GM-CSF via the JAK/STAT pathway. IL-37 also inhibits the NF-κB-mediated activation of S100A9 via STAT3 and p62.

The interleukin IL-27 is a heterodimeric cytokine. IL-27 is primarily secreted by activated macrophages and dendritic cells. The binding of IL-27 to its receptor IL-27R and gp130 leads to the activation of STAT1 and inhibits GATA-3 which further downregulates the expression of IL-4 and reduces mucin production via MUC5AC.

The interleukin IL-35 acts as an anti-inflammatory cytokine which is secreted by T cells and B cells. Studies have found that IL-35 induces proliferation of regulatory T cells, inhibiting CD4⁺ effector cells, and suppressing the development of Th₁₇ cells. Upon binding to IL-12Rβ2/gp130, IL-35 activates the JAK/STAT pathway to inhibits GATA3, thereby regulating the expression of MUC5AC.

The interleukin IL-38 belongs to the interleukin-1 family and plays a significant role in inflammation and immune responses, acting against pathogenic microorganisms. IL-38 has binding affinity for IL-1R and IL-36R and inhibits MAPK-mediated downstream signaling, leading to the decreased activation of cytokines through AP1 and modulating inflammation.

Inhibition of specific inflammatory pathways has therefore been shown to result in the diminished production of pro-inflammatory cytokines such as IL-1α, IL-6, IL-1β, and IL-17. Additionally, the use of IL-6 and IL-1 drugs has been suggested for control of cytokine storms in patients infected with SARS-CoV-2 virus.

It has also been established that inflammation in the mucosa is the main pathophysiological mechanism leading to congestion in a number of respiratory tract diseases; such inflammation is particularly heightened in infections with SARS-CoV-19 virus due to the presence of elevated pro-inflammatory cytokines. The resulting buildup of mucus can also contribute to other complications associated with infections with SARS-CoV-2 virus such as venous engorgement, elevation in volume of nasal secretions, and pulmonary edema.

One alternative for treatment of infections with SARS-CoV-2 virus, addressed in further detail below, is the administration of drugs that inhibit key inflammatory signaling molecules, in particular IL-1β, IL-5, IL-6, and TNF-α, as well as pathways associated with inflammation. Such drugs include the IL-5 inhibitor mepolizumab, the IL-5 inhibitor reslizumab, the IL-5 inhibitor benralizumab, the IL-6 inhibitor sarilumab, the IL-6 inhibitor siltuximab, the IL-6 inhibitor tocilizumab, the IL-1 inhibitor anakinra, the JAK pathway inhibitor tofacitinib, the TNF-α inhibitor infliximab, the IL-17 inhibitor secukinumab, and the IL-1β inhibitor canakinumab.

Another alternative for treatment of infections with SARS-CoV-19 virus, addressed in further detail below, is the administration of drugs that inhibit MUC5AC. Such drugs include niclosamide and carbocisteine.

Infection with SARS-CoV-2 virus can additionally cause dysregulation in other processes associated with the innate immune system and with the interaction between the innate immune system and the adaptive immune system (Y. Li & X. X. Tang, “Abnormal Airway Mucus Secretion Induced by Virus Infection,” Frontiers Immunol. 12: 701443 (2021)). In particular, the first-line signals including interferon (IFN) signals and the NOD-like receptor family pyrin domain containing 3 (NLRP3) inflammasome may be the most dominant subsequent to infection with SARS-CoV-2 virus, which correlates with the uncoordinated IFN responses that amplify TNF-α/IL-1β-centered hyperinflammatory signatures in patients with severe with SARS-CoV-2 virus infections.

Therefore, mucus targeting has emerged as a potential approach in the treatment of patients infected with SARS-CoV-2 virus (S. S. Kumar et al., “Mucus Targeting as a Plausible Approach to Improve Lung Function in COVID-19 Patients,” Med. Hypoth. 156: 110680 (2021)). Existing therapies for mucus targeting include carbocisteine and N-acetylcysteine. Other potential therapies for mucus targeting include: the CXCR2 antagonist AZD5069 (N-[2-[(2,3-difluorophenyl)methylsulfanyl]-6-[(2R,3S)-3,4-dihydroxybutan-2-yl]oxypyrimidin-4-yl]azetidine-1-sulfonamide), which inhibits neutrophil movement and thereby inhibits mucin secretion; EGFR tyrosine kinase inhibitors such as gefitinib and other EGFR inhibitors, including, but not limited to, afatinib, brigatinib, erlotinib, lapatinib, neratinib, olmutinib, osimertinib, rociletinib, and vandetanib; p38 mitogen-activated protein kinase inhibitors such as acumapimod, which inhibit mucin secretion by airway epithelium by inhibiting MARCKs protein; anti-IDO antibody, clone 10.1 (IDO is an enzyme that catalyzes the conversion of tryptophan to kynurenines and is upregulated by interferon γ, by inhibiting this mechanism, the expression of genes encoding MUC proteins via the KYN AhR signaling pathway is downregulated); statins such as simvastatin, which inhibit mucus production and goblet cell hyperplasia; and BIO-11006 acetate ((2S)-2-[[(2S)-2-[[(2S)-2-[[(2S,3R)-2-[[(2S)-2-[[(2S)-2-[[(2S)-2-[[(2S)-2-[[(2S)-2-[(2-acetamidoacetyl)amino]propanoyl]amino]-5-amino-5-oxopentanoyl]amino]-3-phenylpropanoyl]amino]-3-hydroxypropanoyl]amino]-6-aminohexanoyl]amino]-3-hydroxybutanoyl]amino]propanoyl]amino]propanoyl]amino]-6-aminohexanoic acid acetate), which is an analogue of Myristolated N-Terminal Sequence (MANS) and inhibits MARCKS. Blocking interferon signaling may also be a potential strategy to reduce virus-induced airway mucus production and accumulation.

Additionally, mucus hypersecretion may be associated with an increased risk of so-called “long COVID,” which refers to the persistence of many symptoms associated with infection with SARS-CoV-2 virus, such as fatigue, shortness of breath, cough, joint pain, chest pain, difficulty with cognition and concentration, depression, muscle pain, headache, intermittent fever, and heart palpitations (P. Manckoundia & E. Franon, “Is Persistent Thick Copious Mucus a Long-Term Symptom of COVID-19,” EJCRIM 7: doi:102890/2020_002145 (2020)).

As detailed below, one new therapeutic modality for treating infections caused by SARS-CoV-2 and their sequelae is based on the use of nadolol and analogues and derivatives thereof to inhibit mucus hypersecretion. In particular, methods and compositions employing nadolol can be employed.

Particularly, the methods and compositions of the present invention are based on the ability of the β-adrenergic inverse agonist nadolol to inhibit the activity of the arrestin-2 (β-arrestin) pathway, particularly in blocking signaling at airway epithelial cell β₂ receptors.

### I. Properties of Nadolol

The structure of nadolol is shown below as Formula (I):

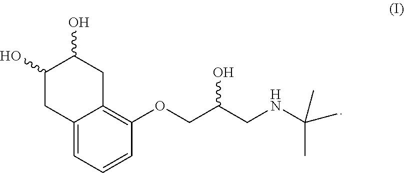 [SMILES: CC(C)(C)NCC(O)COc1cccc2c1CC(O)C(O)C2]

Nadolol is a mixture of four stereoisomers, shown below as Formulas (II(a)), (II(b)), (II(c)), and (II(d)):

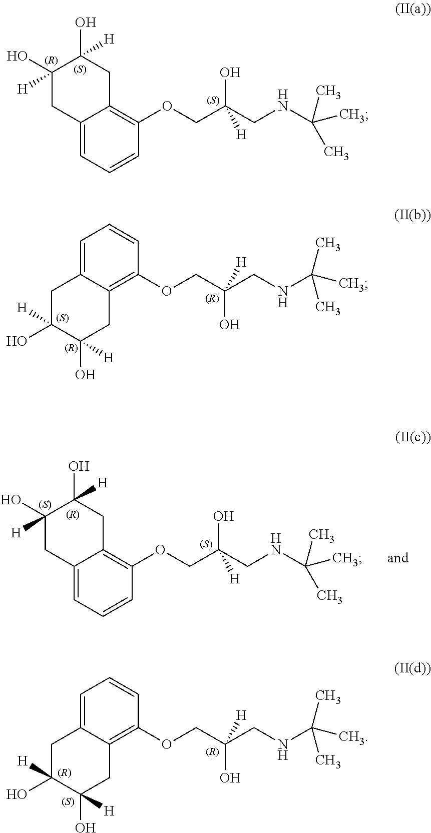

The most active stereoisomer of nadolol is the RSR stereoisomer.

Nadolol is polar and hydrophilic, with low lipid solubility.

As described below, in some alternatives, a derivative or analogue of nadolol can be used. A particular derivative or analogue of nadolol is a compound of Formula (III):

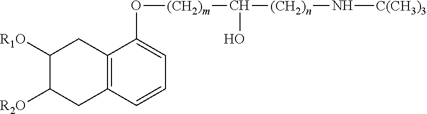

(III), wherein R₁ is hydrogen or lower alkyl, R₂ is hydrogen or lower alkyl, and m and n are 1 to 3, with the proviso that wherein R₁ and R₂ are both hydrogen and m is 1, n is other than 1.

One embodiment of the invention is a method for treating an infection caused by SARS-CoV-19 virus by administering a therapeutically effective quantity of an inverse agonist for a β-adrenergic receptor, particularly a β₂-adrenergic receptor, whose modulation is involved in the disease or condition. As detailed further below, a method according to the present invention particularly treats mucus hypersecretion associated with an infection caused by SARS-CoV-19 virus. Additionally, a method according to the present invention also particularly prevents the occurrence of cytokine storms associated with an infection caused by SARS-CoV-19 virus.

As detailed below, the therapeutic activity of nadolol is based on its inverse agonist activity at β-adrenergic receptors, particularly at β₂-adrenergic receptor, more particularly in the respiratory tract.

In classical receptor theory, two classes of G protein-coupled receptor (GPCR) ligands were considered: agonist and antagonist. Receptors were believed to exist in a single quiescent state that could only induce cellular signaling upon agonist binding to produce an activated receptor state. In this model, binding by antagonists produced no cellular signaling but simply prevented receptors from being bound and activated by agonists. Then, Costa and Herz demonstrated that receptors could be manipulated into a constitutive or spontaneously active state that produced cellular signaling in the absence of agonist occupation. They also provided evidence that certain compounds inactivate those spontaneously active receptors (T. Costa & A. Herz, “Antagonists with Negative Intrinsic Activity at 8 Opioid Receptors Coupled to GTP-Binding Proteins,” Proc. Natl. Acad. Sci. USA 86: 7321-7325 (1989)). There is further evidence that GPCRs exist in constitutively or spontaneously active states that are inactivated to some degree by inverse agonists (R. A. de Ligt et al., “Inverse Agonism at G Protein-Coupled Receptors: (Patho)physiological Relevance and Implications for Drug Discovery,” Br. J. Pharmacol. 130: 1-12 (2000); G. Milligan et al., “Inverse Agonism: Pharmacological Curiosity or Potential Therapeutic Strategy?,” Trends Pharmacol. Sci. 16: 10-13 (2000)).

The basis of the use of inverse agonists such as nadolol to modulate the activity of β-adrenergic receptors, particularly β₂-adrenergic receptors, in general, involves the recognition of the existence of inverse agonists and the understanding of the effect that chronic treatment with inverse agonists has on receptor function. What is an inverse agonist and how does it function? Receptors, such as β-adrenergic receptors (“β-adrenoceptors”) that respond to adrenalin (epinephrine), typically exist in an equilibrium between two states, an active state and an inactive state. When receptors bind to agonists, such as adrenalin for the β-adrenoceptor, they stop them from cycling back into the inactive state, thus shifting the equilibrium between the active and inactive states according to the Law of Mass Action. This occurs because those receptors bound to agonists are removed from the equilibrium. Typically, antagonists bind to the receptors, but prevent the binding of agonists. However, molecules known as “inverse agonists” bind to the receptors in the inactive state, causing the equilibrium between the active and the inactive state to shift toward the inactive state. This is not merely a matter of blocking agonist binding. Moreover, there is a population of spontaneously active receptors in vivo. These receptors provide a baseline constitutive level of activity; the activity is never entirely “off.”

As indicated above, it has been well demonstrated that chronic administration of β-adrenergic agonists causes agonist-dependent desensitization. Upon acute administration of 3-agonists, adrenergic receptors are internalized, thereby preventing them from being restimulated further for pulmonary relaxation. With chronic administration of β-agonists, there is actually a downregulation in the total number of β-adrenergic receptors. The consequence may be the observed loss of responsiveness seen in asthmatic patients or in other patients with chronic respiratory disease on long-acting β-agonists, and referred to as tolerance or tachyphylaxis, as described above.

One aspect of treatment methods according to the present invention is based on the discovery that a chronic administration of an inverse agonist has the effect of upregulating the population of active β-adrenoceptors. The observed activity may be due to the receptor's constitutive baseline activity or the combined effect of increased level of receptors responding to endogenous agonists. This leads to the seemingly paradoxical result that the administration of a drug that would appear, at first blush, to degrade a physiological function, such as by causing airway hyperresponsiveness in asthma, can, if administered chronically, enhance that physiological function by upregulating the population of spontaneously active β-adrenergic receptors associated with that physiological function. This is a specific application of the principle of “paradoxical pharmacology” (R. Lin et al., “Changes in β₂-Adrenoceptor and Other Signaling Proteins Produced by Chronic Administration of “Beta-Blockers” in a Murine Asthma Model,” Pulm. Pharmacol. Ther. 21: 115-124 (2008)), and is particularly applicable to infections caused by SARS-CoV-2 virus affecting the respiratory tract.

Along these lines, the use of cardioselective β-adrenergic inverse agonists (those with a preference for the β₁-adrenergic receptor subtype) has been demonstrated to be safe in hypertensive and congestive heart failure (CHF) patients with chronic obstructive pulmonary disease (COPD).

As stated below, the present invention provides for the use of the active β-adrenoceptor receptor binding forms of β-adrenergic inverse agonists in the treatment of infections with SARS-CoV-2 virus and of sequelae of such infections. A particularly preferred β-adrenergic inverse agonist is nadolol, which, as stated below, has the additional property of inhibiting arrestin-2 (β-arrestin). The inverse agonists can be in pure or substantially pure enantiomeric or diastercomeric form or can be racemic mixtures. In many cases, the active form of such compounds is the L form when the compound has only one chiral center. In the case of nadolol, which has three chiral centers and potentially 12 isomers, though, typically, only two are formed during synthesis, the most active form is the RSR form of nadolol (the designation of chiral centers in nadolol is according to the Cahn-Ingold-Prelog system). As stated above, in some alternatives, a derivative or analogue of nadolol such as, but not limited to, the compound of Formula (III) can be used. As used herein, “analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or hydrophobic or it may have altered reactivity as compared to the parent compound. The analogue may mimic the chemical and/or biological activity of the parent compound (i.e., it may have similar or identical activity), or, in some cases, may have increased or decreased activity. The analogue may be a naturally or non-naturally occurring variant of the original compound. Other types of analogues include isomers (enantiomers, diastereomers, and the like) and other types of chiral variants of a compound, as well as structural isomers. As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have different chemical or physical properties than the parent compound. For example, the derivative may be more hydrophilic or hydrophobic or it may have altered reactivity as compared to the parent compound. Derivatization (i.e., modification) may involve substitution of one or more moieties within the molecule (e.g., a change in functional group). The term “derivative” also includes conjugates and prodrugs of a parent compound (i.e., chemically modified derivatives which can be converted into the original compound under physiological conditions).

Although a number of inverse agonists having activity at β-adrenergic receptors in tissues of the respiratory tract are known, including: nadolol, typically used as the hydrochloride; bupranolol, typically used as the hydrochloride; carazolol, typically used as the hydrochloride; carvedilol, typically used as the hydrochloride; ICI-118,551 (3-(isopropylamino)-1-[(7-methyl-4-indanyl)oxy]butan-2-ol), typically used as the hydrochloride; levobunolol, typically used as the hydrochloride; metoprolol, typically used as the tartrate or succinate; sotalol, typically used as the hydrochloride; and timolol, typically used as the hydrochloride, as well as solvates of these compounds and other salts of these compounds and prodrugs of these compounds, the properties of nadolol make it the preferred compound to be used in methods and compositions of the present invention. The properties of nadolol that make it the preferred compound to be used in methods and compositions according to the present invention include the inhibition of arrestin-2 (β-arrestin) as discussed below.

### II. Arrestin-2 (β-Arrestin)

Arrestins, which include arrestin-2 (β-arrestin), and other members of the family, including arrestin-1, arrestin-3, and arrestin-4, are members of a small family of proteins important for regulating signal transduction at G-protein-coupled receptors (GPCRs). In response to a stimulus, GPCRs activate heterotrimeric G proteins. In order to turn off this response, or to adapt to a persistent stimulus, active receptors need to be sensitized. The first step in desensitization is phosphorylation of G-protein-coupled receptors by a series of serine/threonine kinases called G protein coupled receptor kinases (GRKs). GRK-mediated phosphorylation specifically prepares the activated receptor for arrestin binding. Arrestin binding to the receptor then blocks further G-protein-mediated signaling and targets receptors for internalization, and redirects signaling to alternative G-protein-independent pathways, such as 3-arrestin-mediated signaling. According to J. S. Smith et al., “Biased Signaling: From Simple Switches to Allosteric Microprocessors,” Nat. Rev. Drug Discovery 17: 243-260 (2018), G protein-coupled receptors (GPCRs) are the most common receptors encoded in the genome, comprising greater than 1% of the coding human genome with approximately 800 members and expressed within every organ system. All GPCRs share a common architecture consisting of an extracellular N-terminal sequence, seven transmembrane-spanning (TM) domains (TM1-TM7) that are connected by three extracellular and three intracellular loops, and an intracellular C-terminal domain. GPCRs are sensors of a wide array of extracellular stimuli, including proteins, hormones, small molecules, neurotransmitters, ions, and light. GPCR signaling is primarily controlled by interactions with three protein families: G proteins, G protein receptor kinases (GRKs) and β-arrestins (arrestin-2 proteins) which perform distinct functions at the receptor. Upon stimulation, GPCRs activate heterotrimeric G proteins. Classically, agonist binding causes a conformational change in a GPCR, inducing guanine exchange factor (GEF) activity that catalyzes the exchange of GTP for GDP on Gα subunits of the heterotrimeric G-protein. This, in turn, leads to the dissociation of the heterotrimeric complex into Gα and Gβγ subunits. The dissociated subunits promote the formation of second messenger effectors such as cyclic adenosine monophosphate (cAMP), inositol triphosphate (IP3), diacylglycerol (DAG), and other second messengers, as well as modulation of other receptors and channels, such as activation of inward-rectifying potassium channels.

Similar to most biological systems, negative feedback loops have evolved to quench sustained second messenger signaling following receptor stimulation for maintenance of biological homeostasis. After ligand binding and G protein activation, the receptor is phosphorylated on its cytoplasmic loops and C-terminus, primarily by GRKs, which enhance arrestin-2 binding to the receptor. Arrestins, including arrestin-2, were first discovered for their role in mediating receptor desensitization, the process whereby repeated stimulation decreases the signaling response over seconds to minutes, through steric hindrance of GPCR interaction with the G proteins. Arrestin-2 also mediates receptor internalization via interactions with clathrin coated pits. This can result in downregulation, a sustained decrease in receptor number over minutes to hours due to trafficking of these internalized receptors to proteasomes or lysosomes. Internalized receptors that are not degraded also can be recycled to the plasma membrane. It is now established that in addition to acting as negative regulators of G protein signaling, arrestin-2 also couples to numerous signaling mediators including mitogen-activated protein kinases (MAPKs), AKT, SRC, nuclear factor-κB (NF-κB) and phosphoinositide 3-kinase (PI3K) by acting as adaptors and scaffolds. These pathways are separate from classical G protein signaling, but can involve similar signaling cascades that are often temporally distinct. More recently, it has also been appreciated that some receptors tightly interacting with arrestin-2 maintain catalytic GEF (guanine nucleotide exchange factor) activity on endosomes, continuing to promote G protein signaling after internalization. Thus, arrestin-2 can regulate nearly all aspects of receptor activity, including desensitization, downregulation, trafficking and signaling.

Most drugs that activate or block GPCRs are thought to equally, or substantially equally, target distinct signaling pathways mediated by different G proteins and β-arrestins. These agonists are thought to amplify downstream signaling pathways in a similar fashion to that of the endogenous reference agonist, which are frequently referred to as balanced agonists, while most antagonists are believed to inhibit all second-messenger systems activated by those agonists. However, it was appreciated previously that selective agonists or antagonists could specifically target a particular receptor-linked effector system or a limited number of receptor-linked effector systems. Indeed, a number of ligands have been described that selectively activate some pathways while blocking others downstream of a receptor. Compared with the balanced agonists described above, these “functionally selective” or “biased” agonists can selectively activate G proteins while blocking arrestins, or vice versa. This behavior was initially identified in a number of GPCR systems, including pituitary adenylate cyclase-activating peptide (PACAP) receptor ligands that differentially activated different G proteins as measured by a reversal in potencies. Biased agonism has become an increasingly active area of research since the discovery of β-arrestin-mediated signaling, with a plethora of biased ligands identified for multiple GPCRs.

The discovery of biased agonism has had important implications for understanding of GPCR biology. First, biased agonism is not consistent with two-state models for receptor signaling, and therefore it alters the conventionally understood concept of efficacy. As used herein, the term “efficacy” is defined as the ability of a ligand to generate a quantifiable response after binding to a receptor. The quantifiable response is typically, but not necessarily, a response that promotes a normal physiological function or a treatment for a disease or condition. In the context of this patent application, the disease or condition is infection with the SARS-CoV-2 virus or the consequences of such infection, particularly those that affect the respiratory tract.

Second, biased signaling suggests that GPCRs should not be modelled as binary switches but instead should be modelled as allosteric microprocessors that generate a multitude of conformations in response to different ligands. There are also important clinical implications for these ligands, as selectively activating or inhibiting specific signaling cascades could yield more targeted drugs with reduced side effects. In particular, such targeted drugs could be used to treat infection with the SARS-CoV-2 virus or the consequences of such infection, particularly the consequences that affect the respiratory tract.

The factors that result in the development of a biased signaling response are as follows. Using the ternary complex as a general model for receptor activity, agonist activation of a receptor requires three principal components for initiating signaling: a ligand, a receptor, and at least one transducer or transducers (the number of transducers depends on the particular system). These three (or more in some cases) components act allosterically; for example, a ligand can increase the affinity of a receptor for a transducer with which it interacts, such as a G protein or β-arrestin, while the binding of the transducer to an intracellular receptor domain can stabilize a conformation that increases the affinity for a specific ligand. Allostery is a widespread phenomenon that describes the ability of interactions occurring at a site of a macromolecule to modulate interactions at a spatially distinct binding site on the same macromolecule in a reciprocal manner. The concept of allostery was first developed with regard to enzymes, but is also applicable to many other proteins, particularly, but not exclusively, multi-subunit proteins. As used herein “affinity,” is a measurement of how well a ligand binds to a receptor, commonly expressed in terms of a dissociation constant (K_{d}). The smaller the dissociation constant (expressed in molar units), the more tightly bound the ligand is to the receptor, and thus the higher the affinity between the ligand and the receptor. Affinity depends on cellular context, and therefore, affinity for a G protein-coupled receptor, such as the β₂-adrenergic receptor, is influenced by transducers, such as G proteins or β-arrestin, also known as arrestin-2.

In two-state models of ligand-receptor interaction and signaling, there are only binary conditions for the receptor: the inactive state, which is incapable of signaling, and the active state, which can bind to and activate transducers. The receptor is modelled as a switch, with agonists stabilizing the “on” state and antagonists stabilizing the “off” state. Agonist efficacy can be defined as the ability of a ligand to modify the signaling state of the receptor by stabilizing the active receptor conformation. The phenomenon of biased agonism demonstrates that receptors are not acting as simple switches that merely encode states of activity across a binary spectrum, that is, either agonists or antagonists that equally activate or inhibit all signaling pathways downstream of a receptor. Rather, ligand binding results in the activation or inhibition of multiple GPCR-mediated effectors, including, in this context, β-adrenergic receptors such as the β₂ receptor. These effectors often rely on distinct phases of G protein, GRK and β-arrestin signaling. Instead of encoding binary “on” or “off” signals, a more appropriate model is one where a GPCR acts as an allosteric microprocessor with pluridimensional efficacy, responding to different molecules with different transducer coupling efficiencies. Any site on the receptor surface that binds to a molecule can, in theory, stabilize a distinct receptor conformation and induce a particular pharmacological output. Therefore, the physiological activity of a drug need not necessarily be linked to an interaction at the orthosteric binding site. Any of the three components of a ternary complex as described above, the ligand, the receptor, and the transducer or transducers, can contribute to such a biased response. The general understanding of ligand bias is that it is transmitted through the receptor to downstream transducers as a result of ligand-induced generation of distinct conformations of an allosteric receptor. This may involve participation of a number of mechanisms, including changes in the secondary or tertiary structure of the receptor and the recruitment of proteins that post-translationally modify the intracellular loops and the C-terminal of the receptor, such as by phosphorylation or ubiquitylation.

Arrestin-2 blocks GPCR coupling in two ways. First, arrestin-2 binding to the cytoplasmic face of the receptor occludes the binding site for a member of the class of heterotrimeric G-proteins, thus preventing its activation and resulting in desensitization. Secondly, arrestin-2 links the receptor to elements of the internalization machinery, clathrin (which is a protein that builds small vesicles in order to transport molecules within cells) and clathrin adaptor AP2, which promotes receptor internalization via coated pits and subsequent transport to internal cellular compartments, called endosomes. Subsequently, the receptor can either be directed to degradation compartments (lysosomes) or recycled back to the plasma membrane where it can again participate in signaling. The strength of arrestin-receptor interactions plays a role in which of these alternatives occur: tighter complexes tend to increase the probability of receptor degradation (Class B), while more transient complexes favor recycling (Class A), although the strength of the interaction is not completely determinative.

Arrestins, including arrestin-2, are elongated molecules in which several intramolecular interactions determine the relative origins of the two domains of the protein. In an unstimulated cell, arrestins are localized in the cytoplasm in a basal, “inactive” conformation. Active phosphorylated GPCRs recruit arrestin to the plasma membrane. Receptor binding induces a global conformational change that involves the movement of the two arrestin domains and the release of its C-terminal tail that contains clathrin and AP2 binding sites. Increased accessibility of these sites in receptor-bound arrestin targets the arrestin-receptor complex to the coated pit as described above. Arrestins also bind microtubules (part of the cellular “skeleton”), where they assume yet another conformation, different from both free and receptor-bound form. Microtubule-bound arrestins recruit certain proteins to the cytoskeleton, which affects their activity and/or redirects it to microtubule-associated proteins. Arrestins shuttle between the cell nucleus and the cytoplasm. Their nuclear functions are not fully understood, but it has been shown that all four mammalian arrestin subtypes remove some of their partners, such as protein kinase JNK3 or the ubiquitin ligase Mdm2, from the nucleus. Arrestins also modify gene expression by enhancing transcription of certain genes.

Desensitization of GPCRs, a phenomenon described above that is responsible for the loss of activity of agents such as β-adrenergic agonists typically occurring with chronic administration of such agents, has several mechanisms. Briefly, the classical paradigm for desensitization involves the dual step of receptor phosphorylation by second messenger-stimulated protein kinases (i.e., PKA or PKC, termed heterologous desensitization) or specific G-protein-coupled receptor kinases (GRKs, termed homologous desensitization) and subsequent binding of arrestin to sterically interdict further coupling between the receptor and the G protein. Together, these two actions regulate functional coupling of receptors to effector molecules and control the subcellular localization of the receptor during the process of agonist-induced homologous desensitization. However, another arrestin-2-mediated regulatory mechanism for desensitization involves the degradation of a second messenger, such as adenylyl cyclase-generated cAMP, by scaffolding phosphodiesterases (PDEs) to the vicinity of the effector. Whereas these two desensitization processes were thought in the past to be independent of one another, recruitment of both arrestin-2 and PDE to activated β-adrenoceptors showed very similar kinetics, suggesting that arrestin-2 may serve as shuttles for the translocation of PDE to the activated receptor. This observation was strengthened by the fact that cells lacking arrestin-2 expression were unable to recruit PDE following β-adrenoceptor stimulation, a function that was rescued by the introduction of exogenous arrestin-2 to cells. Arrestin-2-mediated recruitment of PDE to activated β-adrenoceptors was shown in a subsequent study performed in cardiac myocytes to promote the switching from Gs to Gi coupling, thus shifting the receptor toward a pathway that further limits cAMP production, thus blocking further signal transmission by the β-adrenoceptors.

The exposure of agonist results in trafficking of GPCRs into intracellular compartments in a process of sequestration or internalization. Internalization was initially identified as a critical step in resensitization of desensitized receptors but more recently was shown to initiate cellular signaling such as mitogenic pathways. A central role for arrestin-2 in the internalization process was discovered by showing that overexpression of arrestin-2 rescues internalization-deficient β-adrenoceptor mutants impaired in their sequestration ability and conversely that β-adrenoceptor internalization can be inhibited with arrestin-2-defective mutants.

The mechanism by which arrestin-2 mediates receptor internalization is via its ability to interact with proteins of the clathrin-coated pit (CCP) machinery. It is now understood that arrestin-2 functions as an adaptor molecule that binds directly to clathrin via the adaptor protein AP-2. AP-2 recruitment to the plasma membrane is facilitated by a direct agonist-dependent interaction between GRK2 and PI3 kinase in the cytosol, followed by rapid translocation of both enzymes to the plasma membrane where they interact with agonist-activated receptors. The generation of 3,4,5-phosphatidylinositols at the membrane by PI3 kinase enhances the recruitment of AP-2, thus promoting endocytosis. Interestingly, the internalization process also requires protein kinase activity of PI3 kinase, which acts to phosphorylate cytoskeletal tropomyosin, allowing for actin polymerization.

Complex formation between the receptor and the components of the sequestration machinery depends on the phosphorylation state of arrestin-2. In the unstimulated state, arrestin-2 is constitutively phosphorylated by the mitogen-activated protein kinase (MAPK) ERK1/2, thus reducing its capacity to interact with clathrin and thus reducing sequestration of GPCRs. On agonist stimulation of GPCRs such as β-adrenoceptors, cytoplasmic arrestin-2 is recruited to the plasma membrane, where it is rapidly dephosphorylated, an action that promotes its binding to actin and subsequent endocytosis.

Heterotrimeric G proteins constitute a class of membrane-associated G proteins that form a heterotrimeric complex. The biggest non-structural difference between heterotrimeric and monomeric G proteins is that heterotrimeric proteins bind to their cell-surface receptors, called G protein-coupled receptors (GPCRs), directly. These G proteins are comprised of α, β, and γ (alpha, beta, and gamma) subunits. The α subunit is attached directly to either a GTP or a GDP molecule, which serves as an on-off switch for the activation of the G protein. When ligands bind a GPCR, the GPCR acquires GEF (guanine nucleotide exchange factor) ability, which activates the G protein by exchanging the GDP on the α subunit with GTP by releasing the GDP, allowing the binding of GTP. The binding of GTP to the α subunit results in a structural change and its dissociation from the remainder of the G protein. Generally, the α subunit binds membrane-bound effector proteins for the downstream signaling cascade, but the β-γ complex can carry out this function also. G-proteins are involved in pathways such as the cAMP/PKA pathway, pathways involving ion channels, the MAPK signaling pathway, or the PI3K signaling pathway.

Once internalized into intracellular compartments, receptors, such as β-adrenoceptors, are destined to either recycle back to the plasma membrane or, alternatively, become targeted for postendocytic degradation. One of the cellular processes that determines the fate of the receptor is ubiquitination—the adding of multiple ubiquitin molecules to lysine residues of the substrate protein, an action that marks it for degradation by the proteasome. Indeed, several GPCRs, including β-adrenoceptors, undergo agonist-mediated ubiquitination. However, although ubiquitination of the β₂-adrenoceptor is necessary for receptor degradation, concomitantly occurring ubiquitination of arrestin-2 on agonist stimulation is also required for β₂-adrenoceptor internalization. Results with mutants suggest that the ubiquitination status of arrestin-2 determines the stability of the receptor-arrestin-2 complex in addition to its trafficking pattern. Applicant further emphasizes that β₂-adrenoceptors are abundant in the respiratory tract, which is typically the first organ system affected by infection with SARS-CoV-2 virus.

Arrestin-2 also functions as an adaptor molecule that promotes the formation of multi-protein signaling complexes with proteins such as ERK and receptor and nonreceptor tyrosine kinases. This interaction is G-protein-independent and functions to activate mitogenic pathways, such as those mediated by the ERK1/2-MAPK cascade.

The interaction of arrestin-2 with an activated receptor is mediated by the members of the family of GRKs; for the β₂-adrenoceptors, these members include at least GRK5 and GRK6. These GRKs induce receptor phosphorylation that leads to agonist-stimulated ERK activation in the absence of G-protein activation for β₂-adrenoceptors.

In addition, the CXC4 receptor has a role in activating inflammation through activation of arrestin-2-mediated pathways. As noted above, inflammation, particularly, although not exclusively, affecting the respiratory tract, is a hallmark of infection by SARS-CoV-2 virus and also characterizes sequelae of such infection. Infection as a consequence of infection by SARS-CoV-2 virus can also affect other tissues and organs and can occur in long COVID.

These different roles and activities of arrestin-2 are further addressed below in terms of therapeutic methods and compositions, including therapeutic methods and compositions involving proteins with which arrestin-2 has been shown to interact.

### III. Methods of Treatment of Infection by SARS-CoV-2 Virus and Its Sequelae Affecting the Respiratory Tract

Accordingly, one aspect of the present invention is a method for treating infection by SARS-CoV-2 virus and sequelae of such infection affecting the respiratory tract as described above by administration of a therapeutically effective quantity of nadolol or a derivative or analogue thereof to a patient with COPD to inhibit the activity of arrestin-2, thereby preventing the desensitization of β₂-adrenoceptors.

Preferably, the nadolol or derivative or analogue of nadolol is nadolol itself. Alternatively, within the scope of the invention are analogues of nadolol of Formula (III):

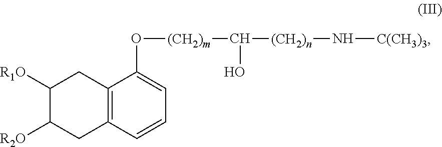

wherein R₁ is hydrogen or lower alkyl, R₂ is hydrogen or lower alkyl, and m and n are 1 to 3, with the proviso that where R₁ and R₂ are both hydrogen and m is 1, n is other than 1. As used herein, the term “lower alkyl” is defined as a straight or branched hydrocarbyl residue of 1 to 6 carbon atoms, more preferably 1 to 3 carbon atoms. Other derivatives or analogues of nadolol are described below.

The derivatives or analogues of nadolol that can be used in methods according to the present invention can include salts where a moiety present in the derivative or analogue of nadolol has one or more groups that can either accept or donate protons, depending on the pH of the solution in which they are present. These moieties include carboxyl moieties, hydroxyl moieties, amino moieties, sulfonic acid moieties, and other moieties known to be involved in acid-base reactions. The recitation of a derivative or analogue of nadolol include such salt forms as occur at physiological pH or at the pH of a pharmaceutical composition unless specifically excluded. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, β-hydroxybutyrates, glycolates, tartrates, methane-sulfonates, propanesulfonates, naphthalene-1-sulfonates, naphthalene-2-sulfonates, and mandelates. If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

Similarly, prodrug esters can be formed by reaction of either a carboxyl, a hydroxyl, or a sulfonic acid moiety on compounds or analogues or derivatives thereof suitable for methods according to the present invention with either an acid (for hydroxyl moieties) or an alcohol (for carboxyl or sulfonic acid moieties) to form an ester. Typically, the acid or alcohol includes a lower alkyl group of 1 to 6 carbons, such as methyl, ethyl, propyl, isopropyl, butyl, isobutyl, and tertiary butyl. These groups can be further substituted with substituents such as hydroxy or other substituents as described above as long as such substituents would not substantially impair the hydrolysis of the prodrug or the bioavailability of the resulting hydrolysis product. Such prodrugs are well known in the art and need not be described further here. The prodrug is converted into the active compound by hydrolysis of the ester linkage, typically by intracellular enzymes. Other suitable moieties that can be used to form prodrug esters are well known in the art. For example, prodrugs can include amides prepared by reaction of the parent acid compound with a suitable amine. In some cases it is desirable to prepare double ester type prodrugs such as (acyloxy) alkyl esters or ((alkoxycarbonyl)oxy)alkyl esters. Suitable esters as prodrugs include, but are not necessarily limited to, methyl, ethyl, propyl, isopropyl, n-butyl, isobutyl, t-butyl, morpholinoethyl, and N,N-diethylglycolamido. Methyl ester prodrugs may be prepared by reaction of the acid form of a compound having a suitable carboxylic acid group in a medium such as methanol with an acid or base esterification catalyst (e.g., NaOH or H₂SO₄). Ethyl ester prodrugs are prepared in similar fashion using ethanol in place of methanol. Morpholinylethyl ester prodrugs may be prepared by reaction of the sodium salt of a suitable compound (in a medium such as dimethylformamide) with 4-(2-chloroethyl)morphine hydrochloride (available from Aldrich Chemical Co., Milwaukee, Wis. USA). The use of prodrug systems is described in T. Jarvinen et al., “Design and Pharmaceutical Applications of Prodrugs” in Drug Discovery Handbook (S. C. Gad, ed., Wiley-Interscience, Hoboken, NJ, 2005), ch. 17, pp. 733-796.

Pharmaceutically acceptable salts include salts as described above. Other pharmaceutically acceptable salts are known in the art.

The subject to be treated can be a human patient or a socially or economically important animal as described above. Methods and compositions according to the present invention are not limited to the treatment of humans unless so specified. As stated above, infection by SARS-CoV-2 virus is not limited to humans, and it has been postulated that infected non-human mammals may form a reservoir for subsequent human infection.

Typically, when sustained-release oral administration of the nadolol, the derivative or analogue of nadolol, or the prodrug of the nadolol or of the derivative or analogue of nadolol is used, the administration results in continuous levels of the nadolol, the derivative or analogue of nadolol, or, in the case of administration of a prodrug, an active agent resulting from the in vivo metabolism of the prodrug in the bloodstream of the subject. Typically, the method exerts a therapeutic effect that is an upregulation of pulmonary β₂-adrenoceptors. Typically, the method also exerts a therapeutic effect that is an inhibition of the activity of arrestin-2 at pulmonary β₂-adrenoceptors. Typically, as described in further detail below, the method also directly blocks the entry of SARS-CoV-2 through the ACE2 receptor by inhibition of arrestin-2 activity. Typically, the method also exerts a therapeutic effect that is increased pulmonary relaxation responsiveness to β₂-adrenergic agonist drugs. This provides for combination therapy, described in detail below. Typically, the nadolol is administered by inhalation, as described in detail below.

The nadolol, the derivative or analogue of nadolol, or the prodrug of the nadolol or the derivative or analogue of nadolol can be administered in conjunction with one or more pharmaceutical excipients, such as in a pharmaceutical composition. The formulation of pharmaceutical compositions is well known in the art. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. The nadolol, the derivative or analogue of nadolol, or the prodrug of the nadolol or the derivative or analogue of nadolol can be administered in conjunction with one or more pharmaceutically acceptable carriers. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. Other pharmaceutical excipients and carriers are known in the art, and include, but are not limited to: preservatives; sweetening agents for oral administration; thickening agents; buffers; liquid carriers; wetting, solubilizing, or emulsifying agents; acidifying agents; antioxidants; alkalinizing agents; carrying agents; chelating agents; colorants; complexing agents; suspending or viscosity-increasing agents; flavors or perfumes; oils; penetration enhancers; polymers; stiffening agents; proteins; carbohydrates; bulking agents; and lubricating agents. The use of such agents for pharmaceutically active substances is well known in the art, and suitable agents for inclusion into dosage forms can be chosen according to factors such as the quantity of nadolol or other active agent to be included per unit dose, the intended route of administration, the physical form of the dosage form, and optimization of patient compliance with administration. Except insofar as any conventional medium, carrier, or agent is incompatible with the active ingredient or ingredients, its use in a composition according to the present invention is contemplated. Supplementary active ingredients can also be incorporated into the compositions, especially as described below under combination therapy. For administration of any of the compounds used in the present invention, preparations should meet sterility, pyrogenicity, general safety, and purity standards as required by the FDA Office of Biologics Standards or by other regulatory organizations regulating drugs.

When a pharmaceutical composition according to the present invention includes a protein, a fragment of a protein, or a peptide, the pharmaceutical composition is formulated so that the protein can be successfully administered. If the pharmaceutical composition is formulated for oral administration, suitable carrier or carriers need to be in the formulation to protect the protein, the fragment of the protein or the peptide from digestive enzymes that would otherwise hydrolyze the protein, the fragment of the protein or the peptide, as well as from acidic pH values in the intestine and other factors interfering with the absorption of these molecules. Various carriers have been described, including protease inhibitors and omega-3 fatty acids (U.S. Pat. No. 10,058,593 to Kidron). Additional carriers and methods are described in M. Nicze et al., “The Current and Promising Oral Delivery Methods for Protein- and Peptide-Based Drugs,” Int. J. Mol. Sci. 25: 815 (2024).

Thus, the nadolol, the derivative or analogue of nadolol, or the prodrug of the nadolol or of the derivative or analogue of the nadolol can be formulated for oral, sustained-release oral, buccal, sublingual, inhalation, insufflation, or parenteral administration.

If the nadolol, the derivative or analogue of nadolol, or the prodrug of the nadolol or of the derivative or analogue of the nadolol is administered orally, either in a conventional or a sustained-release preparation, it is typically administered in a conventional unit dosage form such as a tablet, a capsule, a pill, a troche, a wafer, a powder, or a liquid such as a solution, a suspension, a tincture, or a syrup. Oral formulations typically include such normally employed excipients as, for example, pharmaceutical grades of mannitol, lactose, starch, magnesium stearate, sodium saccharin, cellulose, magnesium carbonate, and other conventional pharmaceutical excipients. In certain defined embodiments, oral pharmaceutical compositions will comprise an inert diluent and/or assimilable edible carrier, and/or they may be enclosed in hard or soft shell gelatin capsules. Alternatively, they may be compressed into tablets. As another alternative, particularly for veterinary practice, they can be incorporated directly into food. For oral therapeutic administration, they can be incorporated with excipients or used in the form of ingestible tablets, buccal tablets, dragees, pills, troches, capsules, wafers, or other conventional dosage forms.

The tablets, pills, troches, capsules, wafers, or other conventional dosage forms can also contain the following: a binder, such as gum tragacanth, acacia, cornstarch, sorbitol, mucilage of starch, polyvinylpyrrolidone, or gelatin; excipients or fillers such as dicalcium phosphate, lactose, microcrystalline cellulose, or sugar; a disintegrating agent such as potato starch, croscarmellose sodium, or sodium starch glycolate, or alginic acid; a lubricant such as magnesium stearate, stearic acid, talc, polyethylene glycol, or silica; a sweetening agent, such as sucrose, lactose, or saccharin; a wetting agent such as sodium lauryl sulfate; or a flavoring agent, such as peppermint, oil of wintergreen, orange flavoring, or cherry flavoring. When the dosage unit form is a capsule, it can contain, in addition to materials of the above types, a liquid carrier. Various other materials can be present as coatings or to otherwise modify the physical form and properties of the dosage unit. For instance, tablets, pills, or capsules can be coated with shellac, sugar, or both. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical preparations for oral use can be obtained by combining the active compound or compounds with solid excipient, optionally grinding a resulting mixture, and processing the mixture of granules, after adding suitable auxiliary components, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses or different doses of a single active compound.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycol. In addition, compatible stabilizers may be added. Such stabilizers do not hinder oral administration.

In one alternative, an extended-release formulation is used. Extended-release formulations are well-known in the art. For example, they can include the use of polysaccharides such as xanthan gum and locust bean gum in conjunction with carriers such as dimethylsiloxane, silicic acid, a mixture of mannans and galactans, xanthans, and micronized seaweed, as disclosed in U.S. Pat. No. 6,039,980 to Baichwal. Other extended-release formulations incorporate a biodegradable polymer, such as the lactic acid-glycolic acid polymer disclosed in U.S. Pat. No. 6,740,634 to Saikawa et al. Still other extended-release formulations incorporate an expandable lattice that includes a polymer based on polyvinyl alcohol and polyethylene glycol, as disclosed in U.S. Pat. No. 4,428,926 to Keith. Still other extended-release formulations are based on the Eudragit™ polymers of Rohm & Haas that include copolymers of acrylate and methacrylates with quaternary ammonium groups as functional groups as well as ethylacrylate methylmethacrylate copolymers with a neutral ester group. A particularly-preferred extended release composition suitable for use in methods according to the present invention is an extended-release composition that contains nadolol as its active ingredient.

Oral liquid preparations can be in the form of, for example, aqueous or oily suspensions, solutions, emulsions, syrups, tinctures, or elixirs, or can be presented as a dry product for reconstitution with water or other suitable vehicles before use. Such liquid preparations can contain conventional additives such as suspending agents, for example, sorbitol syrup, methylcellulose, glucose/sugar syrup, gelatin, hydroxymethylcellulose, carboxymethylcellulose, aluminum stearate gel, or hydrogenated edible fats; emulsifying agents, such as lecithin, sorbitan monooleate, or acacia; non-aqueous vehicles (which may include edible oils), for example, almond oil, fractionated coconut oil, oily esters, propylene glycol, or ethyl alcohol; or preservatives, for example, methylparaben, propylparaben, or sorbic acid. The preparations can also contain buffer salts, flavoring, coloring, or sweetening agents (such as mannitol or another conventional sweetening agent compatible with oral administration) as appropriate.

One skilled in the art recognizes that the route of administration is an important determinant of the rate of efficiency of absorption for any particular therapeutically active compound. For example, the alimentary route, e.g., oral, rectal, sublingual, or buccal, is generally considered the safest route of administration. The delivery of the drugs into the circulation is slow, thus eliminating rapid high blood levels of the drugs that could potentially have adverse acute effects. Although this is considered the safest route of administration, there are several disadvantages. One important disadvantage is that the rate of absorption varies, which is a significant problem if a small range in blood levels separates a drug's desired therapeutic effect from its toxic effect, i.e., if the drug has a relatively low therapeutic index. Also, patient compliance is not always ensured, especially if the rectal route of administration is chosen or if oral administration is perceived by the patient as unpleasant. Furthermore, with oral administration, extensive hepatic metabolism can occur before the drug reaches its target site. Another route of administration is parenteral, which bypasses the alimentary tract. One important advantage of parenteral administration is that the time for the drug to reach its target site is decreased, resulting in a rapid response, which is essential in an emergency. Furthermore, parenteral administration allows for delivery of a more accurate dose. Parenteral administration also allows for more rapid absorption of the drug, which can result in increased adverse effects. Unlike alimentary administration, parenteral administration requires a sterile formulation of the drug and aseptic techniques are essential. The most significant disadvantage to parenteral administration is that it is not suitable for insoluble substances. In addition to alimentary and parenteral administration routes, topical and inhalation administrations can be useful. Topical administration of a drug is useful for treatment of local conditions; however, there is usually little systemic absorption. Inhalation of a drug provides rapid access to the circulation and is the common route of administration for gaseous and volatile drugs, or drugs that can be vaporized or nebulized. It is also a desired route of administration when the targets for the drug are present in the pulmonary system, which is the case for compositions and methods according to the present invention.

A particularly preferable route of administration for nadolol is by inhalation. Typically, administration of nadolol by inhalation comprises administration of a dose administered by use of a pressurized metered dose inhaler (pMDI), dry powder inhaler, or nebulizer; the administration of the dose by inhalation may or may not generate measurable blood levels of nadolol in the range associated by oral dosing. Typically, the inhaled dose will be delivered by pMDI in the range of from about 1% to about 10% of the minimally effective oral dose.

A pressurized metered dose inhaler consists of three major components: the canister which is produced in aluminum or stainless steel by means of deep drawing, where the formulation resides; the metering valve, which allows a metered quantity of the formulation to be dispensed with each actuation; and an actuator (or mouthpiece) which allows the patient to operate the device and directs the aerosol into the patient's lungs. The formulation itself is made up of the drug, a liquefied gas propellant and, in many cases, stabilizing excipients. The actuator contains the mating discharge nozzle and generally includes a dust cap to prevent contamination. To use the inhaler the patient presses down on the top of the canister, with their thumb supporting the lower portion of the actuator. Actuation of the device releases a single metered dose of the formulation which contains the medication either dissolved or suspended in the propellant. Breakup of the volatile propellant into droplets, followed by rapid evaporation of these droplets, results in the generation of an aerosol consisting of micrometer-sized medication particles that are then inhaled. Pressurized metered dose inhalers are disclosed in U.S. Pat. No. 10,806,701 to Bonelli et al. Further details about metered dose inhalers are provided in P. B. Myrdal et al., “Advances in Metered Dose Inhaler Technology: Formulation Development,” AAPS Pharm. Sci. Tech. 15: 434-455 (2014), which discloses alternatives for formulations suitable to be administered by metered dose inhalers.

Dry powder inhalers commonly hold the medication either in a capsule for manual loading or in a proprietary form inside the inhaler. Once the inhaler is loaded or actuated, the operator inserts the mouthpiece of the inhaler into his or her mouth and takes a sharp, deep inhalation, ensuring that the medication reaches the lower parts of the lungs, holding his or her breath for 5-10 seconds. Some powder inhalers use lactose as an excipient. Dry powder inhalers are disclosed in U.S. Pat. No. 10,842,952 to Bilgic.

Nebulizers use oxygen, compressed air, or ultrasonic power to break up solutions or suspensions into small aerosol droplets that are inhaled from the mouthpiece of the device. An aerosol is a mixture of gas and solid or liquid particles. The most common nebulizers are jet nebulizers, sometimes referred to as atomizers. Other forms of nebulizers are soft mist inhalers, ultrasonic wave nebulizers, and vibrating mesh nebulizers. Nebulizers are disclosed in U.S. Pat. No. 10,799,902 to Maeda et al., U.S. Pat. No. 10,786,638 to Alizoti et al., and U.S. Pat. No. 10,716,907 to Eicher et al.

When compounds, such as pharmaceutically acceptable agents such as nadolol, are formulated for parenteral administration, e.g., formulated for injection via the intravenous, intramuscular, subcutaneous, intralesional, or intraperitoneal routes or other routes known in the art, many options are possible. The preparation of an aqueous composition that contains an effective amount of the nadolol, the derivative or analogue of nadolol, or the prodrug of the nadolol or the derivative or analogue of nadolol as an active ingredient will be known to those of skill in the art. Typically, such compositions can be prepared as injectables, either as liquid solutions and/or suspensions. Solid forms suitable for use to prepare solutions and/or suspensions upon the addition of a liquid prior to injection can also be prepared. The preparations can also be emulsified. The pharmaceutical forms suitable for injectable use include sterile aqueous solutions and/or dispersions; formulations including sesame oil, peanut oil, synthetic fatty acid esters such as ethyl oleate, triglycerides, and/or aqueous propylene glycol; and/or sterile powders for the extemporaneous preparation of sterile injectable solutions and/or dispersions. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. In all cases the form must be sterile and/or must be fluid to the extent that the solution will pass readily through a syringe and needle of suitable diameter for administration. It must be stable under the conditions of manufacture and storage and must be preserved against the contaminating action of microorganisms, such as bacteria or fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and use, these preparations contain a preservative to prevent the growth of microorganisms. Suitable non-sensitizing and non-allergenic preservatives are well known in the art.

The carrier can also be a solvent and/or dispersion medium containing, for example, water, ethanol, a polyol (for example, glycerol, propylene glycol, and/or liquid polyethylene glycol, and/or the like), suitable mixtures thereof, and/or vegetable oils. The proper fluidity can be maintained for example, by the use of a coating, such as lecithin, by the maintenance of a suitable particle size in the case of a dispersion, and/or by the use of surfactants. The prevention of the action of microorganisms can be brought about by the inclusion of various antibacterial and/or antifungal agents, for example, parabens, chlorobutanol, phenol, sorbic acid, or thimerosal. In many cases it will be preferable to include isotonic agents, for example, sugars or sodium chloride. In many cases, it is preferable to prepare the solution in physiologically compatible buffers such as Hanks's solution, Ringer's solution, or physiological saline buffer. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by sterilization if desired or necessary. Sterilization is typically performed by filtration. However, alternative methods of sterilization are known in the art, such as by heating or autoclaving, if the ingredients in the solution are stable to such conditions and do not degrade or oxidize under such conditions. Generally, dispersions are prepared by incorporating the various sterilized active ingredients into a sterile vehicle which contains the basic dispersion medium and/or the other required ingredients. In the case of sterile powders for the preparation of sterile injectable solutions, the preferred methods of preparation are vacuum-drying and/or freeze-drying techniques that yield a powder of the active ingredients plus any additional desires ingredients from a previously sterile-filtered solution thereof. The preparation of more-concentrated or highly-concentrated solutions for direct injection is also contemplated, where the use of dimethyl sulfoxide (DMSO) as solvent is envisioned to result in extremely rapid penetration, delivering high concentrations of the active agents to a small area if desired.

For parenteral administration of an aqueous solution, for example, the solution should be suitably buffered if necessary and/or the liquid diluent first rendered isotonic with sufficient saline, glucose, or other tonicity agent. These particular aqueous solutions are especially suitable for intravenous, intramuscular, subcutaneous, or intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected into the proposed site of infusion (see, e.g., “Remington's Pharmaceutical Sciences” (15^{th} ed.), pp. 1035-1038, 1570-1580). Some variation in dosage will necessarily occur depending on the condition of the subject being treated. The person responsible for administration will, in any event, determine the appropriate dose for the individual subject. Compounds and compositions according to the invention can also be formulated for parenteral administration by bolus injection or continuous infusion and can be presented in unit dose form, for instance as ampules, vials, small volume infusions, or pre-filled syringes, or in multi-dose containers with an added preservative.

Another route of administration of compositions according to the present invention is nasally, using dosage forms such as nasal solutions, nasal sprays, aerosols, or inhalants. Nasal solutions are usually aqueous solutions designed to be administered to the nasal passages in drops or sprays. Nasal solutions are typically prepared so that they are similar in many respects to nasal secretions, so that normal ciliary action is maintained. Thus, the aqueous nasal solutions usually are isotonic and/or slightly buffered in order to maintain a pH of from about 5.5 to about 6.5. In addition, antimicrobial preservatives, similar to those used in ophthalmic preparations, and/or appropriate drug stabilizers, if required, can be included in the formulation. Various commercial nasal preparations are known and can include, for example, antibiotics or antihistamines. Spray compositions can be formulated, for example, as aqueous solutions or suspensions or as aerosols delivered from pressurized packs, with the use of a suitable propellant, such as dichlorodifluoromethane, trichlorofluoromethane, dichlorotetrafluoroethane, 1,1,1,2,3,3,3-heptafluoropropane, 1,1,1,2-tetrafluoroethane, carbon dioxide, or other suitable gas. In many alternatives according to the present invention, nasal administration is preferred as that route of administration advantageously provides access to the respiratory tract, which, as stated above, is a major site of inflammation caused by infection with SARS-CoV-2 virus.

Additional formulations that are suitable for other modes of administration include vaginal suppositories and/or pessaries. A rectal pessary or suppository can also be used. Suppositories are solid dosage forms of various weights or shapes, usually medicated, for insertion into the rectum, vagina, or urethra. After insertion, suppositories soften, melt, and/or dissolve into the cavity fluids. In general, for suppositories, conventional binders or carriers can include polyalkylene glycols, cocoa butter, or triglycerides.

Other dosage forms, including but not limited to liposomal formulations, ointments, creams, lotions, powders, or creams, can alternatively be used. Ointments and creams can, for example, be formulated with an aqueous or oily base with the addition of suitable gelling agents and/or solvents. Such bases, can thus, for example, include water and/or an oil such as liquid paraffin or a vegetable oil such as arachis (peanut) oil or castor oil or a solvent such as a polyethylene glycol. Thickening agents which can be used include soft paraffin, aluminum stearate, cetostearyl alcohol, polyethylene glycols, microcrystalline wax, and beeswax. Lotions can be formulated with an aqueous or oily base and will in general also contain one or emulsifying agents, stabilizing agents, dispersing agents, suspending agents, or thickening agents.

Powders for external application can be formed with the aid of any suitable powder base, for example, talc, lactose, or starch.

Because of the nature of the interaction between inverse agonists and the 3-adrenergic receptors with which they interact, the therapeutic response develops gradually over time as the receptor density in the affected tissues increases in response to the administration of inverse agonists. Therefore, in one particularly preferred alternative, the dosage is titrated at the start of administration with gradual increases. In other words, the β-adrenergic inverse agonist, specifically the nadolol, the derivative or analogue of nadolol, or the prodrug of the nadolol or the derivative or the analogue of the nadolol, is administered over time in a series of graduated doses starting with the lowest dose and increasing to the highest dose. When the highest dose is reached, the β-adrenergic inverse agonist continues to be administered at that dose (the maintenance dose). For example, with nadolol administered orally, treatment can begin with 1 mg dosages, then progress through 3 mg, 5 mg, 10 mg, 15 mg, and then to higher maintenance dosages such as 25 mg, 30 mg, 50 mg, 70 mg, 100 mg, 150 mg, or higher as deemed necessary, depending on the particular condition to be treated, the severity, and the response of the condition to the treatment. In the context of the present invention, the “particular condition to be treated” is an infection caused by SARS-CoV-2 virus or sequelae of such an infection, particularly respiratory sequelae of such an infection.

Accordingly, another aspect of the invention is a blister pack that includes a range of dosages from the lowest initial dose to the highest maintenance dose of the nadolol, the derivative or analogue of nadolol, or the prodrug of the nadolol or the derivative or analogue of the nadolol. In general, such a blister pack comprises:
(1) a lower substrate;
(2) an intermediate dosage holder that is shaped to generate a plurality of cavities and that is placed over the lower substrate, the cavities being shaped to hold dosage forms of the nadolol, the derivative or analogue of nadolol, or the prodrug of the nadolol or the derivative or analogue of the nadolol;
(3) an upper substrate placed over the intermediate dosage holder that has a plurality of apertures, each aperture being located to accommodate a corresponding cavity; wherein the dosage forms are of graduated dosages starting with a lowest dose and proceeding to a highest dose; and
(4) dosage forms of the nadolol, the derivative or analogue of nadolol, or the prodrug of the nadolol or of the derivative or analogue of the nadolol, placed in the cavities.

When the blister pack includes doses of nadolol, the doses of nadolol can be selected from the alternatives of 1 mg, 3 mg, 5 mg, 10 mg, 15 mg, 30 mg, 50 mg, and 70 mg per unit dose. In some alternatives, particularly where the blister pack is designed for administering nadolol to patients in which the maximum dosage of nadolol has been reached, the blister pack can contain multiple doses of the same amount of nadolol, such as 50 mg or 70 mg per unit dose.

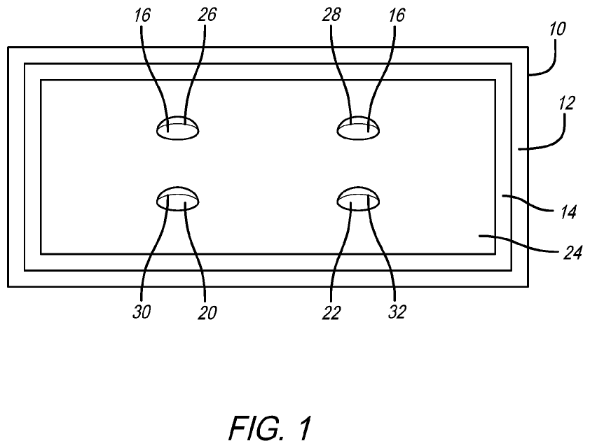

A suitable blister pack 10 is shown in FIG. 1 and includes a lower substrate 12 that is typically foil, an intermediate dosage holder 14 that is shaped to generate a plurality of cavities 16, 18, 20, and 22 shaped to hold the pills, capsules, or other dosage forms that is placed over the lower substrate, and an upper substrate 24 placed over the intermediate dosage holder 14 that has apertures 26, 28, 30, and 32, each aperture being located to accommodate the cavities 16, 18, 20, and 22. Only four cavities and apertures are shown here, but blister packs 10 according to the present invention can hold a larger number of dosage forms, such as 10, 20, or 30. Typically, either the lower substrate 12, the upper substrate 24, or both have printed instructions on it to identify the dosage of each pill, capsule, or other dosage forms, and to provide guidance to the patient as to the sequence to be followed in taking the pills, capsules, or other dosage forms. The intermediate dosage holder 14 is typically made of a transparent plastic or other transparent material so that the dosage forms can be viewed. The dosage forms can be of graduated doses, starting with a lowest dose and proceeding to a highest dose, which is generally the maintenance dose, as described above. Alternatively, the dosage forms can be of at least two dosages: (1) a maintenance dose that is the highest in a series of graduated doses; and (2) at least one backup restoration dose (to be used, e.g., if a dose is missed) or a lower dose to be taken in a specified condition. The specified condition can be, for example, the administration of an antibiotic, such as erythromycin or neomycin or other therapeutic agent, such as an antiviral agent, where lower dosages may be required or when kidney or liver malfunction increases the half-life of the drug necessitating a lower dose to achieve the same serum concentration when kidney and liver function were both normal.

Various factors must be taken into account in setting suitable dosages for the nadolol, the derivative or analogue of the nadolol, or the prodrug of the derivative or the analogue of the nadolol. These factors include whether the patient is taking other medications that can alter the pharmacokinetics of the nadolol, the derivative or analogue of the nadolol, or the prodrug of the derivative or the analogue of the nadolol, either causing them to be degraded more rapidly or more slowly. These medications can, for example, affect either liver or kidney function or may induce the synthesis of one or more cytochrome P450 enzymes that can metabolize the nadolol, the derivative or analogue of the nadolol, or the prodrug of the derivative or the analogue of the nadolol. In particular, if the patient is taking the antibiotics erythromycin or neomycin or certain other medications, it is typically necessary to decrease the maintenance dose. Another aspect of the invention is therefore a blister pack that has backup restoration doses and lower doses for use when the patient is taking these antibiotics or other medications that reduce the rate of catabolism of the nadolol, the derivative or analogue of the nadolol, or the prodrug of the derivative or the analogue of the nadolol. In another alternative, when the patient is taking a medication that induces the synthesis of one or more cytochrome P450 enzymes that can metabolize the nadolol, the derivative or analogue of the nadolol, or the prodrug of the derivative or the analogue of the nadolol, a blister pack according to the present invention can include one or more higher doses for temporary use.

Toxicity and therapeutic efficacy of β-adrenergic inverse agonists used in methods and compositions according to the present invention, in particular, nadolol or a derivative or analogue of nadolol, or a prodrug of the derivative or the analogue of the nadolol, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀; compounds which exhibit large therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays. For example, a dose can be formulated in animal models to achieve a circulating plasma concentration range that includes the IC₅₀ as determined in cell culture (i.e., the concentration of the test compound which achieves a half-maximal improvement in receptor signaling when chronic effects are considered). Such information can be used to more accurately determine useful doses in humans. Levels in plasma may be measured, for example, by HPLC or other methods known in the art, such as gas chromatography.

The exact formulation, route of administration and dosage can be chosen by the individual physician in view of the patient's condition. (See, e.g., Fingl et al., in The Pharmacological Basis of Therapeutics, 1975, ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated, in particular, infection by the SARS-CoV-19 virus or sequelae of such infection, and to the route of administration. The severity of the condition may, for example, be evaluated, in part, by standard prognostic evaluation methods. Further, the dose and perhaps the dose frequency or the dose duration, will also vary according to the age, body weight, and response of the individual patient. A program comparable to that discussed above may be used in veterinary medicine. As stated above, the SARS-CoV-19 virus has been shown to infect mammals other than humans.

Depending on the specific conditions being treated, such agents may be formulated and administered systemically or locally. Typically, administration is systemic. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; parenteral delivery, including intramuscular, subcutaneous, or intramedullary injections, as well as intrathecal, direct intraventricular, intravenous, intraperitoneal, intranasal, or intraocular injections, just to name a few. Typically, oral administration or administration by inhalation, as addressed above, is preferred for administration of nadolol, derivatives or analogues of nadolol, or prodrugs of the nadolol or the derivatives or analogues of nadolol, for treatment of conditions, in particular, infection by the SARS-CoV-19 virus or sequelae of such infection.

For injection, the agents of the invention may be formulated in aqueous solutions. For transmucosal administration, penetrants appropriate to the barrier to be permeated are used in the formulation. Such penetrants are generally known in the art. The most important class of penetrants is surfactants, including bile salts such as sodium deoxycholate, fatty acids such as oleic acid, and phospholipids (J. K. W. Lam et al., “Transmucosal Drug Administration as an Alternative Route in Palliative and End-of-Life Care During the COVID-19 Pandemic,” Adv. Drug Deliv. Rev. 160: 234-243 (2020)).

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Typically, in methods according to the present invention, the inverse agonist, in particular, the nadolol, the derivative or analogue of nadolol, or the prodrug of the nadolol or the derivative or prodrug of the nadolol, is administered in a daily dose or multiple times per day, depending on the half-life of the inverse agonist. Alternatively, the inverse agonist can be administered less frequently, such as every other day, every third day, every fourth day, every week, and the like. One skilled in the art of pharmacokinetics will recognize the importance of understanding the bioavailability and the half-life of a drug in relation to dosing of the particular drug. It is well known that a drug accumulates in the body if the time interval between doses is less than four of its half-lives, in which case, the total body stores of the drug are increased exponentially to a plateau or steady-state concentration. The average total body store of a drug at the plateau is a function of the dose, the interval between doses, the bioavailability of the drug, and the rate of the elimination of the drug. Thus, one of ordinary skill in the art is capable of determining the dose and interval of the dose for a given drug to achieve the desired effect.

Additionally, the activity of nadolol in blocking the activity of arrestin-2 can function to directly block the entry of SARS-CoV-2 virus into susceptible cells, including cells of the respiratory tract (M. S. Maginnis, “β-Arrestins and G Protein-Coupled Receptor Kinases in Viral Entry: A Graphical Review,” Cell Signal 102: 110558 (2023)).

Viruses rely on host-cell machinery in order to invade host cells and carry out a successful infection. G-protein coupled receptor (GPCR)-mediated signaling pathways are master regulators of cellular physiological processing and are an attractive target for viruses to rewire cells during infection. In particular, the GPCR-associated scaffolding proteins β-arrestins and GPCR signaling effectors G-protein receptor kinases (GRKs) have been identified as key cellular factors that mediate viral entry and orchestrate signaling pathways that reprogram cells for viral replication. Interestingly, a broad range of viruses have been identified to activate and/or require GPCR-mediated pathways for infection, including, but not limited to SARS-CoV-2 virus, demonstrating that such viruses may have conserved mechanisms of host-cell invasion. Thus, GPCR-mediated pathways highlight an attractive target for the development of broad antiviral therapies.

GPCRs are seven-transmembrane spanning receptors that transmit signals from extracellular stimuli into intracellular signals that exert essential physiological effects for cellular function. Once the receptor is activated by a ligand such as a neurotransmitter, chemokine, or a pathogen like a virus, the GPCR recruits intracellular effectors that bind to the intracellular loops of the receptor, such as heterotrimeric G proteins, GRKs, or β-arrestins. Specifically, GPCRs are phosphorylated by GRKs, priming the receptor for recruitment of β-arrestins, which serve as scaffolding proteins for endocytosis. The binding of β-arrestins leads to further recruitment of endocytic proteins, including AP2 and clathrin. The recruitment of β-arrestins also leads to activation of mitogen-activated protein kinases (MAPKs). In addition to priming GPCRs through phosphorylation, the members of the GRK family are responsible for agonist-driven receptor desensitization or recycling and activation of other cellular proteins through phosphorylation.

Specifically, with respect to SARS-CoV-2 virus, SARS-CoV-2 infection is initiated by interactions with multiple cellular receptors including angiotensin-converting enzyme 2 (ACE2) receptor (J. Yang et al., “Molecular Interaction and Inhibition of SARS-CoV-2 Binding to the ACE2 Receptor,” Nature Commun. 11: 4541 (2020)). Viral entry occurs through clathrin-mediated endocytosis and deposition of virions in cellular lysosomes in the endocytic pathway (C. B. Jackson et al., “Mechanisms of SARS-CoV-2 Entry into Cells,” Nat. Rev. Mol. Cell. Biol. 23: 3-20 (2022)). The β-arrestins bind to the ACE2 receptor with high affinity and facilitate ACE2 receptor-mediated internalization (Q. Zhang et al., “ACE2 Interaction with Cytoplasmic PDC Protein Enhances SARS-CoV-2 Invasion,” iScience 24: 102770 (2021)). The binding of β-arrestins to ACE2 receptor is supported through a PDZ domain in Na⁺/H⁺ exchanger regulatory factor 1 (NHERF1) that interacts with the PDZ-binding domain in ACE2 receptor, resulting in a super-complex and enhancing the internalization of SARS-CoV-2 virus (S. P. Nisar et al., “Arrestin Scaffolds NHEFR1 to the P2Y12 Receptor to Regulate Receptor Internalization,” J. Biol. Chem. 287: 20405-20415 (2012); R. Yan et al., “Structural Basis for the Recognition of SARS-CoV-2 by Full-Length Human ACE2,” Science 1444-1448 (2020)). In addition, a role for GRKs has recently been described for SARS-CoV-2 infection through GRK-mediated phosphorylation of the viral N protein (B. Chatterjee & S. S. Thakur, “Infection Triggers Phosphorylation: Potential Target for Anti-COVID-19 Therapeutics,” Front. Immun. 13: 829474 (2022)). Therefore, the method of administering a therapeutically effective quantity of nadolol or a derivative or analogue of nadolol to inhibit the β-arrestin pathway exerts a therapeutic effect that is a blockage of the ability of the SARS-CoV-2 virus to infect cells via the ACE2 receptor.

Another embodiment of the present invention is methods and compositions that incorporate multiple-drug or combination therapy for the treatment of infections caused by SARS-CoV-2 virus or sequelae of such infections. Patients with pulmonary airway diseases, including infections caused by SARS-CoV-2 virus or sequelae of such infections, which typically affect the respiratory tract, often are prescribed multiple drugs that work in combination to control their symptoms.

Although Applicant does not intend to be bound by this theory, it is believed that, in many circumstances, co-treatment with an inverse agonist, particularly the nadolol, the derivative or the analogue of the nadolol, or the prodrug of the nadolol or the derivative or the analogue of the nadolol, and with an agonist is superior to treatment with the agonist alone. These results suggest that co-treatment with the inverse agonist may increase the therapeutic efficacy of the agonist and prevent desensitization of the relevant GPCR. One rationale for this form of combination therapy may lie in the treatment of acute episodes or exacerbations of the disease or condition. Even if treatment with inverse agonists decreases the frequency of such acute episodes or exacerbations, there is still a need to treat the acute episode or exacerbation. This can be done by co-administration of the inverse agonist and the agonist. This treatment modality can be applied to treatment of infections caused by SARS-CoV-2 virus or sequelae of such infections.

In one particularly desirable combination, the β-adrenergic inverse agonist is administered in combination with β₂-selective adrenergic agonists for the treatment of infections caused by SARS-CoV-2 virus or sequelae of such infections. The β₂-selective adrenergic agonists are typically selected from the group consisting of abediterol, arformoterol, bambuterol, bitolterol, broxaterol, carbuterol, carmoterol, clenbuterol, colterol, dobutamine, fenoterol, formoterol, indicaterol, isoprenaline, isoxsuprine, levabuterol, mabuterol, metaproterenol, methoxyphenamine, navafenterol, olodaterol, pirbuterol, procaterol, ritodrine, salbutamol, salmeterol, terbutaline, vilanterol, and zilpaterol, as well as the salts, solvates, and prodrugs thereof. These agents vary with respect to their duration of action, generally being classified as short-acting, long-acting, or ultra-long-acting agents. Particularly preferred β₂-selective adrenergic agonists for use in combination with nadolol include isoproterenol, salbutamol, and salmeterol. The principle of combination therapy is supported by the data that shows that treatment with inverse agonists causes upregulation of the receptor number. In that case, co-treatment with an agonist would be expected to increase cellular signaling and restore normal function in those circumstances in which the pathological response is characterized by a deficiency in signaling. Along these lines, the inhibitory response of inverse agonists on airway resistance would be increased in magnitude by the co-administration of agonists. The potency of these agonists may be reduced due to the presence of the inverse agonist, but the overall magnitude of the response would be increased. This would prevent the desensitization often associated with chronic agonist treatment.

When combination therapy is used, the dosages of each member of the combination can be determined according to the principles described above. However, in many cases, fixed dose combinations are desirable and can be used. In the fixed dose combinations, the dosages of the β-adrenergic inverse agonists are as described above, while the desirable dosage of the β₂-selective adrenergic agonist can be determined as described above. The determination of the dosages of each member of the combination or the use of fixed dose combinations also apply to other combinations of β-adrenergic inverse agonists and other therapeutic agents as described below.

In another desirable combination, β-adrenergic inverse agonists are administered together with corticosteroids. Corticosteroids include, but are not necessarily limited to, beclomethasone, budesonide, ciclesonide, deflazacort, flunisolide, fluticasone, methylprednisolone, mometasone, prednisolone, prednisone, betamethasone, dexamethasone, triamcinolone, acrocinonide, alclometasone, amcinafal, amcinafide, amebucort, amelometasone, benzodrocortisone, butixocort, chloroprednisone, ciclometasone, ciprocinonide, clobetasol, clorcotolone, cormetasone, cortobenzolone, deprodone, descinolone, dexbudesonide, dimesone, domoprednate, doxibetasol, drocinonide, endrisone, etiprednol, fluazacort, flucorolone, flumetasone, flumoxonide, fluocinolone, fluocinonide, fluocortin, fluocortolone, 9α-fluorocortisone, fluorometholone, flupamesone, fluperolone, fluprednidene, halcinonide, halocortolone, halopredone, halocortamate, hydrocortisone, icometasone, isoflupredone, isoprednidene, itrocinonide, locicortolone, mazipredone, meclorisone, meprednisone, naflocort, nicocortonide, nivacortol, oxisopred, paramethasone, prednylidene, procinonide, resocortol, rofleponide, ticabesone, timobesone, tipredane, tixocortol, tralonide, triclonide, and the salts, solvates, and prodrugs thereof. The corticosteroids especially preferred for use according to the invention include, but are not necessarily limited to, betamethasone, beclomethasone, budesonide, ciclesonide, flunisolide, fluticasone, methylprednisolone, prednisolone, prednisone, dexamethasone, and triamcinolone, and the salts, solvates, and prodrugs thereof. In particular, betamethasone and dexamethasone are useful in combination with the β-adrenergic inverse agonist for treatment of infections caused by SARS-CoV-2 virus or sequelae of such infections.

In yet another desirable combination, β-adrenergic inverse agonists are administered together with anticholinergic drugs. The anticholinergic drugs especially preferred for use according to the invention include, but are not necessarily limited to, muscarinic receptor antagonists, especially quaternary ammonium muscarinic receptor antagonists such as ipratropium bromide, tiotropium bromide, oxitropium bromide, aclidinium bromide, glycopyrronium bromide, umeclidinium bromide, and abediterol, and the salts, solvates, and prodrugs thereof.

In yet another desirable combination, β-adrenergic inverse agonists are administered together with a xanthine compound. Xanthine compounds include theophylline, extended-release theophylline, aminophylline, theobromine, enprofylline, diprophylline, isbufylline, choline theophyllinate, albifylline, arofylline, bamifylline, caffeine, 8-chlorotheophylline, diprophylline, doxofylline, enprofylline, etamiphylline, furafylline, 3-isobutyl-1-methylxanthine, IBMX (1-methyl-3-(2-methylpropyl)-7H-purine-2,6-dione), MRS-1706 (N-(4-acetylphenyl)-2-[4-(2,3,6,7-tetrahydro-2,6-dioxo-1,3-dipropyl-1H-purin-8-yl)phenoxy]acetamide), ambuphylline, proxyphylline, and xanthinol, and the salts, solvates, and prodrugs thereof. Xanthine compounds especially preferred for use according to the invention include, but are not necessarily limited to, theophylline, extended-release theophylline, aminophylline, theobromine, enprofylline, diprophylline, isbufylline, choline theophyllinate, albifylline, arofylline, bamifylline, ambuphylline, 8-chlorotheophylline, doxofylline, furafylline, IBMX (1-methyl-3-(2-methylpropyl)-7H-purine-2,6-dione), MRS-1706 (N-(4-acetylphenyl)-2-[4-(2,3,6,7-tetrahydro-2,6-dioxo-1,3-dipropyl-1H-purin-8-yl)phenoxy]acetamide), proxyphylline, and caffeine, and the salts, solvates, and prodrugs thereof.

In yet another desirable combination, β-adrenergic inverse agonists are administered together with an anti-IgE antibody. As used herein, unless further defined or limited, the term “antibody” encompasses both polyclonal and monoclonal antibodies, as well as genetically engineered antibodies such as chimeric, humanized or fully human antibodies of the appropriate binding specificity. As used herein, unless further defined, the term “antibody” also encompasses antibody fragments such as sFv, Fv, Fab, Fab′ and F(ab)′₂ fragments. In many cases, it is preferred to use monoclonal antibodies. In some contexts, antibodies can include fusion proteins comprising an antigen-binding site of an antibody, and any other modified immunoglobulin molecule comprising an antigen recognition site (i.e., antigen-binding site) as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins, therapeutic agents, antimetabolites, or radioisotopes; in some cases, conjugation occurs through a linker or through noncovalent interactions such as an avidin-biotin or streptavidin-biotin linkage. Typically, the anti-IgE antibody is a monoclonal antibody or a genetically engineered antibody that is derived from a monoclonal antibody. Preferably, the anti-IgE antibody is humanized. A particularly preferred humanized anti-IgE antibody is an IgG1κ monoclonal antibody that specifically binds to human IgE and is marketed under the name of omalizumab.

In yet another desirable combination, β-adrenergic inverse agonists are administered together with a leukotriene antagonist. Leukotriene antagonists include, but are not necessarily limited to, ablukast, tipelukast, montelukast, pranlukast, and zafirlukast, and the salts, solvates, and prodrugs thereof.

In yet another desirable combination, β-adrenergic inverse agonists are administered together with a phosphodiesterase IV inhibitor. Phosphodiesterase IV inhibitors include, but are not necessarily limited to, apremilast, atizoram, filaminast, mufemilast, roflumilast, cilomilast, piclamilast, and ibudilast, and the salts, solvates, and prodrugs thereof. The phosphodiesterase IV inhibitors especially preferred according to the present invention include, but are not necessarily limited to, roflumilast, cilomilast, piclamilast, and ibudilast, and the salts, solvates, and prodrugs thereof. Phosphodiesterase IV is the predominant isoform of phosphodiesterase in the lung.

In yet another desirable combination, β-adrenergic inverse agonists are administered together with a 5-lipoxygenase inhibitor. The 5-lipoxygenase inhibitors especially preferred according to the present invention include, but are not limited to, meclofenamate sodium, zileuton, and fenleuton, and the salts, solvates, and prodrugs thereof.

In yet another desirable combination, β-adrenergic inverse agonists are administered together with a mast cell stabilizer. Mast cell stabilizers include, but are not necessarily limited to, selected from the group consisting of bepotastine, alcaftadine, azelastine, cromoglicic acid, ketotifen, lodoxamide, nedocromil, olopatadine, and pemirolast, and the salts, solvates, and prodrugs thereof. The mast cell stabilizers especially preferred according to the present invention include, but are not limited to, azelastine, cromoglicic acid, ketotifen, lodoxamide, nedocromil, olopatadine, and pemirolast, and the salts, solvates, and prodrugs thereof.

In yet another desirable combination, β-adrenergic inverse agonists are administered together with a biological (B. L. Walker & R. Leigh, “Use of Biologicals as Immunotherapy in Asthma and Related Diseases,” Expert Rev. Clin. Immunol. 4: 753-756 (2008)). Typically, the at least one biological is selected from the group consisting of an anti-IL-4 antibody, an anti-IL-13 antibody, an inhibitor of both IL-4 and IL-13, an anti-IL-5 antibody, and an anti-IL-8 antibody. Anti-IL4 antibodies include, but are not limited to, a humanized anti-IL-4 monoclonal antibody, pasolizumab. Anti-IL-13 antibodies include, but are not limited to, a human anti-IL-13 monoclonal antibody, tralokinumab. An inhibitor of both IL-4 and IL-13 is the monoclonal antibody dupilumab, which is a monoclonal antibody that binds to the α subunit of the interleukin-4 receptor (IL4Rα), which modulates the signaling of both the IL-4 and the IL-13 pathways; it therefore acts as a receptor antagonist (A. L. Kau & P. E. Korenblat, “Anti-Interleukin 4 and 13 for Asthma Treatment in the Era of Endotypes,” Curr. Opin. Allergy Clin. Immunol. 14: 570-575 (2014)). Although the invention of the present invention is directed to treatment of infections caused by SARS-CoV-2 virus or sequelae of such infections rather than asthma, such infections and their sequelae as well as asthma are characterized by inflammation affecting the respiratory tract. Anti-IL-5 antibodies include, but are not limited to, the monoclonal antibodies benralizumab, mepolizumab, and reslizumab. Anti-IL-8 antibodies include, but are not limited to, the human monoclonal antibody BMS-986253.

In yet another desirable combination, β-adrenergic inverse agonists are administered together with MAPK kinase inhibitors. MAPK inhibitors, include, but are not limited to, PD 184352 (2-(2-chloro-4-iodoanilino)-N-(cyclopropylmethoxy)-3,4-difluorobenzamide), neflamapimod, SB 202190 (4-[4-(4-fluorophenyl)-5-pyridin-4-yl-1H-imidazol-2-yl]phenol hydrochloride), anisomycin, PD 98059 (2-(2-amino-3-methoxyphenyl)chromen-4-one), SB 203580 (4-[4-(4-fluorophenyl)-2-(4-methylsulfinylphenyl)-1H-imidazol-5-yl]pyridine hydrochloride), U0126 (1,4-diamino-2,3-dicyano-1,4-bis(2-aminophenylthio)butadiene), AG 126 (2-[(3-hydroxy-4-nitrophenyl)methylidene]propanedinitrile), apigenin, 5-iodotubercidin, olomoucine, iso-olomoucine, N⁹-isopropyl olomoucine, PD 169316 (4-[4-(4-fluorophenyl)-2-(4-nitrophenyl)-1H-imidazol-5-yl]pyridine), SB 202474 (4-[5-ethyl-2-(4-methoxyphenyl)-1H-imidazol-4-yl]pyridine), SB 202190 (4-[4-(4-fluorophenyl)-5-pyridin-4-yl-1H-imidazol-2-yl]phenol hydrochloride), SB 202474 (4-[5-ethyl-2-(4-methoxyphenyl)-1H-imidazol-4-yl]pyridine dihydrochloride), adezmapimod, SB 203580 sulfone (4-[4-(4-fluorophenyl)-2-(4-methylsulfonylphenyl)-1H-imidazol-5-yl]pyridine), SB 220025 (4-[5-(4-fluorophenyl)-3-piperidin-4-ylimidazol-4-yl]pyrimidin-2-amine), SC 68376 (2-methyl-4-phenyl-5-pyridin-4-yl-1,3-oxazole), SKF-86002 (6-(4-fluorophenyl)-5-pyridin-4-yl-2,3-dihydroimidazo[2,1-b][1,3]thiazole), U0124 ((2Z,3Z)-2,3-bis[amino(methylsulfanyl)methylidene]butanedinitrile), and ZM 336372 (3-(dimethylamino)-N-[3-[(4-hydroxybenzoyl)amino]-4-methylphenyl]benzamide) (U.S. Pat. No. 7,998,507 to Bosch et al.). Other MAPK inhibitors include doramapimod, ralimetinib dimesylate, PH-797804 (3-[3-bromo-4-[(2,4-difluorophenyl)methoxy]-6-methyl-2-oxopyridin-1-yl]-N,4-dimethylbenzamide), VX-702 (6-(N-carbamoyl-2,6-difluoroanilino)-2-(2,4-difluorophenyl)pyridine-3-carboxamide), losmapimod, SB 269063 (4-[4-(4-fluorophenyl)-5-(2-methoxypyrimidin-4-yl)imidazol-1-yl]cyclohexan-1-ol), BMS-582949 (4-[5-(cyclopropylcarbamoyl)-2-methylanilino]-5-methyl-N-propylpyrrolo[2,1-f][1,2,4]triazine-6-carboxamide), skepinone-L, TAK-715 (N-[4-[2-ethyl-4-(3-methylphenyl)-1,3-thiazol-5-yl]pyridin-2-yl]benzamide), pamapimod, pexmetinib, SD 0006 (1-[4-[3-(4-chlorophenyl)-4-pyrimidin-4-yl-1H-pyrazol-5-yl]piperidin-1-yl]-2-hydroxyethanone), and R1487 (6-(2,4-difluorophenoxy)-8-methyl-2-(oxan-4-ylamino)pyrido[2,3-d]pyrimidin-7-one hydrochloride).

The route of administration of the β-adrenergic inverse agonist and of the additional therapeutic agent can be chosen by one of ordinary skill in the art to optimize therapeutic efficiency, as described above. However, in one preferred alternative, both the β-adrenergic inverse agonist and the additional therapeutic agent are administered by inhalation. In another preferred alternative, the β-adrenergic inverse agonist is administered orally, while the additional therapeutic agent is administered by inhalation. The administration of the additional therapeutic agent by inhalation is typically preferred because of possible toxicity of some of these additional therapeutic agents. However, other routes are possible.

Aerosol therapy allows an almost ideal benefit to risk ratio to be achieved because very small doses of inhaled medication provide optimal therapy with minimal adverse effects. A variety of additional therapeutic agents suitable for use in methods according to the present invention are available in aerosol formulation, including β₂-adrenergic agonists, corticosteroids, and anticholinergics. However, the therapeutic efficiency of drugs administered by aerosolization depends not only on the pharmacological properties of the drugs themselves, but also on the characteristics of the delivery device. The characteristics of the delivery device influence the amount of drug deposited in the lungs and the pattern of drug distribution in the airways.

Aerosols are airborne suspensions of fine particles. The particles may be solids or liquids. Aerosol particles are heterodisperse (i.e. the particles are of a range of sizes) and aerosol particle size distribution is best described by a log normal distribution. Particles tend to settle (sediment), adhere to each other (coagulate), and adhere to structures such as tubing and mucosa (deposit). The particles delivered by aerosol can be conveniently characterized on the basis of their aerodynamic behavior. One parameter is the mass median aerodynamic diameter (MMAD). By definition, a particle distribution with an MMAD of 1 μm has the same average rate of settling as a droplet of unit density and 1 μm diameter.

The size of an aerosol particle, as well as variables affecting the respiratory system, influence the deposition of inhaled aerosols in the airways. On one hand, particles larger than 10 μm in diameter are unlikely to deposit in the lungs. However, particles smaller than 0.5 μm are likely to reach the alveoli or may be exhaled. Therefore, particles that have a diameter of between 1 μm and 5 μm are most efficiently deposited in the lower respiratory tract. Particles of these sizes are most efficient for the delivery of therapeutic agents for diseases and conditions such as infections caused by SARS-CoV-2 virus or sequelae of such infections.

The percentage of the aerosol mass contained within respirable droplets (i.e., droplets with a diameter smaller than 5 μm), depends on the inhalation device being used. Slow, steady inhalation increases the number of particles that penetrate the peripheral parts of the lungs. As the inhaled volume is increased, the aerosol can penetrate more peripherally into the bronchial tree. A period of breath-holding, on completion of inhalation, enables those particles that have penetrated to the lung periphery to settle into the airways via gravity. Increased inspiratory flow rates, typically observed in patients with acute asthma, result in increased losses of inhaled drug. This occurs because aerosol particles impact in the upper airway and at the bifurcations of the first few bronchial divisions. Other factors associated with pulmonary airway disease may also alter aerosol deposition. Airway obstruction and changes in the pulmonary parenchyma are often associated with pulmonary deposition in the peripheral airways in patients with diseases or conditions affecting the respiratory tract, such as infections caused by SARS-CoV-2 virus or sequelae of such infections.

In aerosol administration, the nose efficiently traps particles before their deposition in the lung; therefore, mouth breathing of the aerosolized particles is preferred. The aerosolized particles are lost from many sites. Generally, the amount of the nebulized dose reaching the small airways is <15%. In many cases, approximately 90% of the inhaled dose is swallowed and then absorbed from the gastrointestinal tract. The small fraction of the dose that reaches the airways is also absorbed into the blood stream. The swallowed fraction of the dose is, therefore, absorbed and metabolized in the same way as an oral formulation, while the fraction of the dose that reaches the airways is absorbed into the blood stream and metabolized in the same way as an intravenous dose.

When drugs are administered topically (via aerosol delivery to the lungs), the desired therapeutic effects depend on local tissue concentrations, which may not be directly related to plasma drug concentrations. If a sufficiently large dosage of any drug is given, systemic activity can easily be demonstrated with any inhaled β₂-agonists or corticosteroid. This has several implications. First, for the selection of a drug to be inhaled, topical drugs must combine a high intrinsic activity within the target organ and rapid inactivation of the systemically absorbed drug. Secondly, fewer systemic adverse effects should be expected with drugs that have a low oral bioavailability (whether due to poor gastrointestinal absorption or high first-pass hepatic metabolism). Because most inhaled drugs are administered at a low dosage and have a low oral bioavailability, plasma concentrations of these drugs are much lower than after oral administration. Furthermore, factors influencing pulmonary absorption should be considered. It was recently demonstrated that terbutaline was absorbed through the lung more rapidly in healthy smokers than in healthy nonsmokers. This may affect the onset of action of the drug. It has also been found that the bioavailability of inhaled salbutamol in 10 patients with cystic fibrosis was greater than that in healthy adults. One proposed mechanism for this difference in bioavailability is that the chronically diseased tracheobronchial tree in patients with cystic fibrosis results in higher permeability of salbutamol in this tissue.

Finally, the absolute pulmonary bioavailability of inhaled drugs is difficult to assess because blood concentrations are low, and pulmonary and oral absorption should be distinguished from absorption due to inhalation for pulmonary bioavailability to be determined as accurately as possible. Charcoal can be used to adsorb the swallowed fraction of inhaled terbutaline to discriminate the pulmonary absorption of the drug. Recently, it was shown that a urine collection during the 30 minutes after inhalation of salbutamol represents the amount of drug delivered to the lungs. This technique may be applicable for the determination of bioavailability of other inhaled drugs. Other techniques for the determination of bioavailability of inhaled drugs are also known in the art; these include pharmacodynamic methods using FEV1 measurements, lung deposition studies using radiolabeled formulations, or pharmacokinetic studies using predominantly urinary excretion measurements.

Therapeutic aerosols are commonly produced by atomization of liquids within jet nebulizers or by vibration of a standing pool of liquid (ultrasonic nebulization). Preformed aerosols may also be administered. Examples of the latter include MDIs and dry powder devices. Whatever delivery device is used, patients should be taught to use it correctly.

All jet nebulizers work via a similar operating principle, represented by the familiar perfume atomizer. A liquid is placed at the bottom of a closed container, and the aerosol is generated by a jet of air from either a compressor or a compressed gas cylinder passing through the device. Ultrasonic nebulizers produce an aerosol by vibrating liquid lying above a transducer at frequencies of about 1 mHz. This produces a cloud of particles that is carried out of the device to the patient by a stream of air. Aerosols varying in quantity, size and distribution of panicles can be produced by nebulizers, depending upon the design of the nebulizer and how it is operated. It should be noted that not all nebulizers have the required specifications (MMAD, flow, output) to provide optimum efficacy. A recent study compared the lung deposition from 4 nebulizers in healthy volunteers and showed that median lung aerosol deposition, expressed as percentages of the doses initially loaded into the nebulizers, ranged from 2 to 19%. Nebulized aerosols are particularly useful for children under 5 years of age and in the treatment of severe asthma or chronic obstructive pulmonary disease where respiratory insufficiency may impair inhalation from an MDI or dry powder inhaler. To minimize adverse effects, pH and osmolarity of the nebulized solution should be controlled.

Metered dose inhalers (MDIs), because of their convenience and effectiveness, are probably the most widely used therapeutic aerosol used for inhaled drug delivery to outpatients. Most MDIs in current use contain suspensions of drug in propellant. There are 2 major components of an MDI: (i) the canister, a closed plastic or metal cylinder that contains propellant, active medication, and the metering chamber; and (ii) the actuator, a molded plastic container that holds the canister and directs the released aerosol towards the patient's airway.

Propellant mixtures are selected to achieve the vapor pressure and spray characteristics desired for optimal drug delivery. Chlorofluorocarbons were previously used, but non-chlorinated propellants are now employed because of environmental concerns. Finely divided particles of drug, usually less than 1 μm, are suspended in the pressurized (liquefied) propellant. To prevent the drug from coagulating, a surface-active agent such as sorbitan oleate, lecithin or oleic acid is typically added; other surface-active agents are known in the art. Metering chambers ordinarily contain 25 to 100 μL. The contents of the metering chamber are released when the canister is depressed into the actuator. Almost instantaneously, the propellants begin to evaporate, producing disintegration of the discharged liquid into particles that are propelled forward with great momentum. For optimal pulmonary drug deposition, the medication should be released at the beginning of a slow inspiration that lasts about 5 seconds and is followed by 10 seconds of breath-holding. Several inhalation aids have been designed to improve the effectiveness of an MDI. These are most useful in patients who have poor hand-to-breath coordination. A short tube (e.g. cones or spheres) may direct the aerosol straight into the mouth or collapsible bags may act as an aerosol reservoir holding particles in suspension for 3 to 5 seconds, during which time the patient can inhale the drug. However, when any of these devices is used, aerosol velocity upon entering the oropharynx is decreased and drug availability to the lungs and deposition in the oropharynx is decreased.

Dry powder inhalers have been devised to deliver agents to patients who have difficulty using an MDI (such as children and elderly patients). In general, the appropriate dosage is placed in a capsule along with a flow aid or filler such as large lactose or glucose panicles. Inside the device, the capsule is initially either pierced by needles (e.g., Spinhaler®) or sheared in half (e.g., Rotohaler®). During inhalation the capsule rotates or a propeller is turned, creating conditions that cause the contents of the capsule to enter the inspired air and be broken up to small particles suitable for delivery to the airways. The energy required to disperse the powder is derived from the patient's inspiratory effort. Recently, more convenient multidose dry powder inhalers have been introduced (e.g. Diskhaler®, Turbuhaler®). Potential problems associated with dry powder inhalers include esophageal irritation and, consequently, cough due to the direct effect of powder in airways. Furthermore, the walls of the capsule may be coated with drug as a result of either failure of the capsule to release the drug or failure of the aggregated powder to break up. This may cause virtually all of the drug to be deposited in the mouth. These powder devices do not contain chlorofluorocarbons and may provide an alternative to MDIs.

The clinical use of aerosols for treatment of diseases and conditions affecting the respiratory tract such as, but not limited to, chronic obstructive pulmonary disease and asthma, has been proposed for several compounds proposed herein as additional therapeutic agents, including β₂-agonists and corticosteroids. Such techniques could readily be adapted by one of ordinary skill in the art to the treatment of infections caused by SARS-CoV-2 virus or sequelae of such infections by modifying factors such as dosages and frequencies of administration for such drugs based on the severity of the infections or sequelae and the response to the drugs, as well as the ability of the affected tissues to absorb the drugs, among other factors known in the art.

For β₂-agonists, limited pharmacokinetic data are available in humans mostly because the low dosages of inhaled drugs required for therapeutic activity produce drug concentrations in body fluids that are below assay limits. Little is known about pulmonary bioavailability of those drugs. It is generally argued that an average of 10% of an inhaled dose reaches the lung when given by a MDI. The mean pulmonary bioavailability of terbutaline from an MDI was reported to be 9.1%. When the oral component (swallowed fraction of the dose) was added, the value rose to 16.5%, i.e., an increase of 6.7%. The drugs salmeterol and formoterol have different mechanisms of action underlying their prolonged duration of bronchodilatory effect (12 to 18 hours). Salmeterol appears unique because it has a long side chain that anchors the β₂-agonist molecule to the receptor. Formoterol appears to be an extremely potent classical β₂-agonist. The elimination half-life of formoterol after inhalation was calculated to be between 1.7 and 2.3 hours on the basis of urinary excretion data. A glucuronic acid conjugate was identified. However, it is possible that formoterol has a prolonged elimination half-life that is yet to be detected in humans. Salmeterol is formulated as the xinafoate (hydroxynaphthoic acid) salt. Little is known about the pharmacokinetic properties of this drug. Salmeterol is extensively metabolized by hydroxylation, with the majority of a dose being eliminated predominantly in the feces within 72 hours. The hydroxynaphthoic acid part of the molecule accumulates in plasma during repeated administration as a consequence of its long elimination half-life (12 to 15 days).

For anticholinergic agents, the parent compound of this class is atropine. Synthetic agonists of the muscarinic receptors of acetylcholine are quaternary ammonium compounds and, therefore, cross membrane barriers with difficulty. Because systemic absorption of atropine after inhalation of the drug is nearly complete, this route of administration can produce significant systemic toxicity (Harrison et al. 1986). Ipratropium bromide is the only well studied representative of this class. Absorption through the gastrointestinal tract is slow, as peak plasma concentrations have been recorded 3 hours after oral intake of the drug. The absolute bioavailability after oral intake is only 30%. Elimination of metabolized drug occurs in the urine and bile. Whatever the route of administration, the mean elimination half-life is about 3.5 hours. Plasma concentrations observed with inhaled ipratropium were a thousand times lower than those observed with an equipotent bronchodilatory dose administered orally. This explains why systemic anticholinergic effects do not occur following inhalation of therapeutic doses of ipratropium bromide. These properties are probably shared by other quaternary ammonium anticholinergic agents such as oxitropium bromide, an alternative as described above.

Corticosteroids are frequently administered by inhalation, which can prevent some of the adverse effects usually associated with systemic corticosteroid therapy. To produce a compound with marked topical activity, some of the hydroxyl groups in the hydrocortisone molecule were substituted with acetonide or ester groups. Topically active corticosteroid drugs used for the treatment of patients with respiratory diseases or conditions include beclomethasone, betamethasone valerate, budesonide, triamcinolone, fluticasone and flunisolide; dexamethasone has also been used for the treatment of sequelae of infection by SARS-CoV-2 virus. Of these, beclomethasone and budesonide are the most extensively used in general for treatment of respiratory diseases or conditions. The results of numerous clinical studies have shown that there is little difference between the efficacy of beclomethasone and budesonide. Oropharynx deposition is reduced by using a spacing device, and the development of candidiasis can be prevented by mouth rinsing. Plasma clearance of budesonide was calculated to be 84±27 L/h, which is about 10-fold higher than the average clearance of prednisolone. As a consequence of this high clearance, the elimination half-life of budesonide is short (2.8±1.1 hours). The systemic availability of the swallowed fraction is 10.7±4.3%, indicating that there is extensive first-pass metabolism. Stereoselective metabolism was demonstrated and plasma clearance of the two enantiomers, when calculated on a per kilogram of bodyweight basis, were about 50% higher in 6 children with asthma than in 11 healthy adults. Therefore, administration of budesonide by inhalation should reduce the risk of systemic adverse effects compared with administration of the drug orally. Lung esterases are known to hydrolyze beclomethasone. The absorbed beclomethasone and esterase-hydrolysis products (beclomethasone 17-propionate and beclomethasone) are rapidly converted to less active metabolites during passage through the liver. First-pass hepatic metabolism of the systemically absorbed fluticasone is almost complete, and therefore the inhaled drug has a favorable pharmacokinetic profile. Little data has been published regarding the pharmacokinetic properties of flunisolide, triamcinolone, and betamethasone valerate.

To ensure maximal effects from inhaled drugs, the pharmacological characteristics of the drugs, the physical characteristics of pharmaceutical compositions including the drugs, and the device used to aerosolize the drugs should all be considered. With respect to β₂-agonists, different formulations, with different pulmonary disposition techniques, are available, such as for MDI administration, for administration with a dry powder inhaler, or a solution for nebulization. A unit dose from a dry powder inhaler is twice that release from an MDI, but they have equivalent bronchodilatory effects. The characteristics of the devices vary. For a metered-dose inhaler, typically 12-40% of the dose is deposited in the lung, but up to 80% in the oropharynx. When an MDI is used with a spacer, typically about 20% of the dose is deposited in the lung, but only up to 5% in the oropharynx; thus, the use of a spacer can reduce the proportion of the drug that is deposited in the oropharynx. For a dry powder inhaler, typically 11-16% of the dose is deposited in the lung and 31-72% in the oropharynx. For a nebulizer, typically 7-32% of the dose is deposited in the lung and 1-9% is deposited in the oropharynx. One of ordinary skill in the art can ensure that the proper inhalation therapy device is used and can prepare suitable instructions. Considerations for the use of inhalation therapy are described in A.-M. Tabaret & B. Schmit, “Pharmacokinetic Optimisation of Asthma Treatment,” Clin. Pharmacokinet. 26: 396-418 (1994).

For all of these combinations, the invention further encompasses blister packs that contain either a fixed-dose combination of the β-adrenergic inverse agonist and the additional therapeutic agent, such as the β₂-selective adrenergic agonist, the corticosteroid, the anticholinergic agent, the xanthine compound, the anti-IgE antibody, the leukotriene antagonist, the phosphodiesterase-4 inhibitor, the 5-lipoxygenase inhibitor, the mast cell stabilizer, or the biological; or, in separate pills, capsules, or other dosage forms, the β-adrenergic inverse agonist and the additional therapeutic agent as described above. The use of these blister packs is appropriate when oral administration of the inverse agonist and additional therapeutic agent is desired. The blister packs follow the general design described above and in FIG. 1, and typically include appropriate instructions to the patient or to the physician or other administrator of the medication. In general, when a fixed-dose combination is used, the blister pack comprises:
(1) a lower substrate;
(2) an intermediate dosage holder that is shaped to generate a plurality of cavities and that is placed over the lower substrate, the cavities being shaped to hold dosage forms of the pharmaceutical composition described above containing a β-adrenergic inverse agonist and at least one additional therapeutic agent as described above;
(3) an upper substrate placed over the intermediate dosage holder that has a plurality of apertures, each aperture being located to accommodate a corresponding cavity; and;
(4) dosage forms of the pharmaceutical composition placed in the cavities.

When the β-adrenergic inverse agonist and the additional therapeutic agent are to be administered in separate dosage forms, the blister pack, in general, comprises:
(1) a lower substrate;
(2) an intermediate dosage holder that is shaped to generate a plurality of cavities and that is placed over the lower substrate, the cavities being shaped to hold dosage forms of: (a) a first pharmaceutical composition that comprises: (i) a therapeutically effective amount of a β-adrenergic inverse agonist; and (ii) a first pharmaceutically acceptable carrier; and (b) a second pharmaceutical composition that comprises: (i) a therapeutically effective amount of a second therapeutic agent effective to treat a pulmonary airway disease, the second therapeutic agent being selected from the group consisting of a β₂-selective adrenergic agonist, a corticosteroid, a anticholinergic agent, a xanthine compound, an anti-IgE antibody, a leukotriene antagonist, a phosphodiesterase-4 inhibitor, a 5-lipoxygenase inhibitor, a mast cell stabilizer, and a biological and (ii) a second pharmaceutically acceptable carrier;
(3) an upper substrate placed over the intermediate dosage holder that has a plurality of apertures, each aperture being located to accommodate a corresponding cavity; and;
(4) dosage forms of the first and second pharmaceutical compositions placed in the cavities.

The dosage forms of the first and second pharmaceutical compositions are as described above. Typically, in this arrangement, the dosage forms of the first pharmaceutical composition include dosages starting at a low dose and including a range of dosages up to the highest, maintenance, dose. Other dosage form arrangements are possible depending on the second therapeutic agent or the characteristics of the patient being treated.

Other arrangements are possible for the blister packs, depending on the range of doses to be included and the particular additional agents to be included; factors to be considered include the stability of the additional agents, the form of the dosage of the additional agents to be administered, and the number of doses of the a first pharmaceutical composition that comprises: (i) a therapeutically effective amount of a β-adrenergic inverse agonist; and (ii) a first pharmaceutically acceptable carrier and of the second pharmaceutical composition to be included in the blister pack.

In yet another alternative according to the present invention, the nadolol, the derivative or analogue of nadolol, or the prodrug of the nadolol or the derivative or analogue of the nadolol is administered together with a therapeutically effective quantity of an arrestin-2 inhibitor. As used herein, the term “arrestin-2 inhibitor” refers to any compound that directly or indirectly blocks one or more effects of arrestin-2 on β-adrenergic receptors, particularly β₂-adrenergic receptors, and thus potentiates the activity of such receptors when bound to agonists. For example, U.S. Pat. No. 10,172,907 to Lymperopoulos et al. discloses protein fragments of arrestin-2 as arrestin-2 inhibitors, particularly a fragment with the sequence: Glu-Thr-Pro-Val-Asp-Thr-Asn-Leu-Ile-Glu-Leu-Asp-Thr-Asn-Asp-Asp-Asp-Ile-Val-Phe-Glu-Asp-Phe-Ala-Arg-Gln-Arg-Leu-Lys-Gly-Met- Lys-Asp-Asp-Lys-Asp-Glu-Glu-Asp-Asp-Gly-Thr-Gly-Ser-Pro-His-Leu-Asn-Asn-Arg (SEQ ID NO: 1). Other inhibitors of arrestin-2 are disclosed subsequently.

U.S. Pat. No. 9,926,275 to Thakur et al. discloses small molecule inhibitors of arrestin-2, including compounds of Formula (A-I):

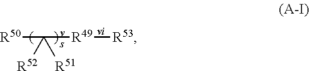

wherein:
(1) v and vi designate the particular bonds indicated in Formula (A-I);
(2) R⁴⁹ is selected from Formulas (A-I(a)), (A-I(b)), (A-I(c)), and (A-I(d)):

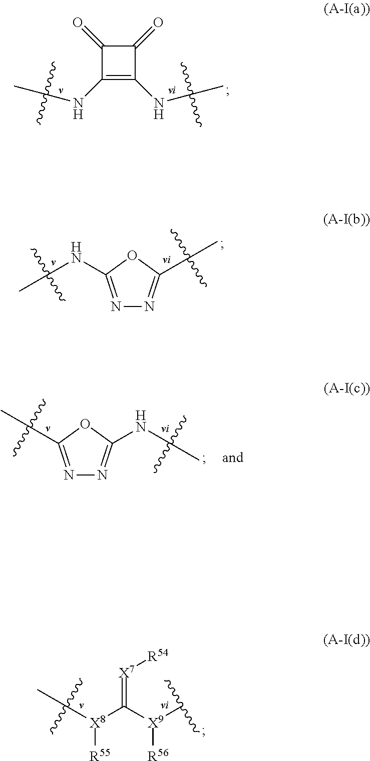

wherein X⁷, X⁸, and X⁹ are each independently O, N, or S;
(2) R⁵⁴, R⁵⁵, and R⁵⁶ are each independently H, cyano, amino, or a substituted or unsubstituted alkyl, alkanoyl, alkanoyloxy, or aryl group when X⁷, X⁸, or X⁹ are respectively N and are absent when X⁷, X⁸, or X⁹ are respectively O or S;
(3) R⁵⁰ is a substituted or unsubstituted aryl or heteroaryl group;
(4) R⁵¹ and R⁵² are each independently H or a substituted or unsubstituted alkyl group, or R⁵¹ and R⁵² together form a 3- or 4-membered cycloalkyl ring;
(5) R⁵³ is a substituted aryl group where one and only one of the substituents is a moiety of Formula (A-I(e)):

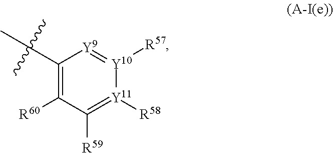

(i) wherein one of R⁵⁷ or R⁵⁸ is a moiety of Formula (A-I(f)):

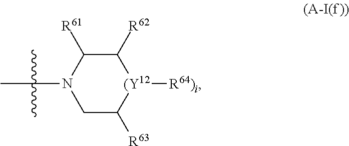

and the other is H, azido, trifluoromethyldiazirido, isocyano, isothiocyano, pentafluorosulfanyl, or a substituted or unsubstituted alkyl, alkanoyl, alkanoyloxy, aryloyl, or aryloyloxy group;
(ii) R⁵⁹ and R⁶⁰ are each independently H, halo, azido, trifluoromethyldiazirido, isocyano, isothiocyano, pentafluorosulfuryl, or a substituted or unsubstituted alkyl, alkanoyl, alkanoyloxy, aryloyl, or aryloyloxy group;
(iii) Y⁹ is CH;
(iv) Y¹⁰ and Y¹¹ are each independently C or N, provided that when Y¹⁰ or Y¹¹ is N then R⁵⁷ or R⁵⁸ respectively is absent;
(v) Y¹² is CH, N, O, S, S(O), or S(O)₂;
(vi) R⁶¹, R⁶², and R⁶³ are each independently H, azido, trifluoromethyldiazirido, isocyano, isothiocyano, or a substituted or unsubstituted alkyl group;
(vii) R⁴ is H or a substituted or unsubstituted alkyl group when Y¹² is CH or N and is absent when Y¹² is O, S, S(O), or S(O)₂; and
(viii) t is 0 or 1; and
(6) s is 0 or 1.

U.S. Pat. No. 8,987,332 to Olefsky et al. discloses omega-3 fatty acids as inhibitors of arrestin-2, including DHA (docosahexaenoic acid) and EPA (eicosapentaenoic acid).

United States Patent Application Publication No. 2016/0311911 by Sur et al., discloses that CXCR2 inhibitors can inhibit the activity of arrestin-2 due to the interaction between CXCR2 and arrestin. CXCR2 inhibitors include, but are not limited to: SB225002 (N-(2-bromophenyl)-N′-(2-hydroxy-4-nitrophenyl)urea), AZD5069 (N-(2-((2,3-difluorobenzyl)thio)-6-(((2R,3S)-3,4-dihydroxybutan-2-yl)oxy)pyrimidin-4-yl)azetidine-1-sulfonamide); SB265610 (1-(2-bromophenyl)-3-(4-cyano-1H-benzo[d][1,2,3]triazol-7-yl)urea); navarixin; danirixin; CXCR2-IN-1 (1-(2-chloro-3-fluorophenyl)-3-[4-chloro-2-hydroxy-3-(1-methylpiperidin-4-yl)sulfonylphenyl]urea); SRT3109 (N-(2-((2,3-difluorobenzyl)thio)-6-((3,4-dihydroxybutan-2-yl)amino)pyrimidin-4-yl)azetidine-1-sulfonamide); and SRT3190 (N-[2-[(2,3-difluorophenyl)methylsulfanyl]-6-[[(2S,3R)-3,4-dihydroxybutan-2-yl]amino]pyrimidin-4-yl]azetidine-1-sulfonamide).

Another category of inhibitors of arrestin is MyD88 inhibitors. MyD88 inhibitors include, but are not limited to: ST2825 ((4R,7R,8aR)-1′-[2-[4-[[2-(2,4-dichlorophenoxy)acetyl]amino]phenyl]acetyl]-6-oxospiro[3,4,8,8a-tetrahydro-2H-pyrrolo[ 2,1-b][1,3]thiazine-7,2′-pyrrolidine]-4-carboxamide) and T6167923 (4-β-bromophenyl)sulfonyl-N-(1-thiophen-2-ylethyl)piperazine-1-carboxamide).

Yet another category of arrestin-2 inhibitors is MD2 inhibitors. MD2 inhibitors include, but are not limited to L48H37 ((3E,5E)-1-ethyl-3,5-bis[(2,3,4-trimethoxyphenyl)methylidene]piperidin-4-one).

United States Patent Application 2004/0053852 by Stamler et al. discloses methods for preventing desensitization of G-protein coupled receptors. These G-protein coupled receptors (GPCRs) include β-adrenergic receptors as well as α-adrenergic receptors, opioid receptors, and prostaglandin receptors. The GPCRs have G-protein receptor kinases (GRKs) associated with them. The GRKs phosphorylate agonist-occupied receptors, thereby promoting binding of β-arrestin molecules, including arrestin-2, which inhibit interactions between the receptors and their associated G-proteins, while also promoting internalization of the receptors. The activity of GRKs thus dampens signaling by the GPCRs. The typical response is decreased levels of GPCRs and the desensitization of the GPCRs, in other words, inability of the agonist that normally binds to the specific GPCR to activate the receptor, which, in certain circumstances, can lead to the inability of the GPCRs to control a disease event associated with lack of activity of a particular GPCR or multiple GPCRs. Nitric oxide donors (NO donors) that donate nitric oxide or a related redox species and provide bioactivity that is identified with nitric oxide, preferably S-nitrosoglutathione (GSNO) inhibit the activity of GRKs thereby allowing GPCRs to signal and to be recycled to the cell surface. This prevents desensitization of the GPCRs and thereby allows GPCRs to be available and active in sufficient quantity to control a disease event that is associated with the lack of activity of a particular GPCR or multiple GPCRs.

NO donors include C-nitroso compounds in which the nitroso moiety is attached to a tertiary carbon, such as the compounds disclosed in U.S. Pat. No. 6,359,182 to Stamler et al. These compounds include C-nitroso compounds having a molecular weight ranging from about 225 to about 1000, or from about 225 to about 600 for oral administration, on a monomeric basis wherein a nitroso group is attached to a tertiary carbon, which is obtained by nitrosylation of a carbon acid having a pKₐ of less than about 25. The compound is preferably water-soluble and preferably contains a carbon atom a to the nitrosylated carbon which is part of a ketone group. In some alternatives, the compound is obtained by nitrosylation of a carbon acid having a pKₐ of less than 10, and, for such compounds, the activity can be potentiated by glutathione. In one alternative, for such compounds, a substituent Q is attached to the tertiary carbon and consists of a chain moiety containing from 1 to 12 chain atoms consisting of 1 to 10 carbon atoms, 0 to 2 nitrogen atoms, and 0 to 2 oxygen atoms covalently bonded to a cyclic moiety which is monocyclic, bicyclic, tricyclic, tetracyclic, or pentacyclic, and contains 5 to 24 ring atoms, consisting of 2 to 20 carbon atoms, 0 to 4 nitrogen atoms, 0 to 1 oxygen atoms, and 0 to 1 sulfur atoms.

In another alternative, the C-nitro compound is a compound of Formula (N-I):

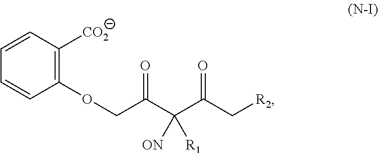

wherein the counterion is hydrogen and wherein R₁ and R₂ are selected from the group consisting of C₁-C₆ alkyl and C₆-C₂₀ aryl, which can be substituted with a substituent selected from the group consisting of amino, hydroxyl, or carboxyl. Such compounds include dimeric 2-[4′-(α-nitroso)isobutyrylphenyl]propionic acid.

Such compounds also include C-nitroso compounds containing a moiety of Formula (N-II):

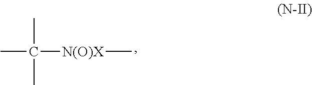

wherein X is S, O, or NR, wherein R is selected from the group consisting of C₁-C₆ alkyl which is unsubstituted or is substituted with one or more alcohol, ether, ester, or amide groups which contain from 1 to 10 carbon atoms; typically, these compounds have a molecular weight of from 100 to about 1000. A preferred subgenus of this alternative comprises the structure

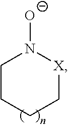

wherein X is S, O, or NR, wherein R is defined as above and n is from 0 to 4; these compounds can alternatively be protonated to remove the negative charge. The structure can be substituted with C₁-C₆ alkyl or C₁-C₆ alkylcarbonyl and can include the modification that the carbon pendant to X in the ring or a carbon within the parenthesis can also be part of another ring.

The compounds include C-nitroso derivatives of acetylsalicylic acid, C-nitroso derivatives of propranolol, C-nitroso derivatives of nadolol, C-nitroso derivatives of carvedilol, C-nitroso derivatives of prazosin, C-nitroso derivatives of tinolol, C-nitroso derivatives of metoprolol, C-nitroso derivatives of pindolol, C-nitroso derivatives of labetalol, C-nitroso derivatives of triamterene, C-nitroso derivatives of furosemide, C-nitroso derivatives of enalapril, C-nitroso derivatives of ramipril, C-nitroso derivatives of lovastatin, C-nitroso derivatives of pravastatin, C-nitroso derivatives of gemfibrozil, C-nitroso derivatives of clofibrate, C-nitroso derivatives of nifedipine, C-nitroso derivatives of amlodipine, C-nitroso derivatives of diltiazem, C-nitroso derivatives of verapamil, C-nitroso derivatives of cimetidine, C-nitroso derivatives of ranitidine, C-nitroso derivatives of albuterol, C-nitroso derivatives of ipratropium bromide, C-nitroso derivatives of memantine, C-nitroso derivatives of 10-deacetylbaccatin III, C-nitroso derivatives of taxol, C-nitroso derivatives of pretomanid, C-nitroso derivatives of dalcetrapib, C-nitroso derivatives of superoxide dismutase mimetics, C-nitroso derivatives of allopurinol, C-nitroso derivatives of celecoxib, C-nitroso derivatives of indomethacin, C-nitroso derivatives of thioflosulide, and C-nitroso derivatives of etodolac.

Inositol hexaphosphate (IP6) is described as an arrestin sequestrant (Y. K. Peterson & L. M. Luttrell, “The Diverse Roles of Arrestin Scaffolds in G Protein-Coupled Receptor Signaling,” Pharmacol. Rev. 69: 256-297 (2017)) and thus has the effect of inhibiting the activity of arrestin-2.

Additionally, it is known that second messenger-stimulated kinases also play a role in the desensitization of GPCR-coupled receptors. For Gₛ-coupled receptors, which include β₁-, β₂-, and β₃-adrenergic receptors, the relevant second messenger-stimulated kinase is cAMP-dependent protein kinase, also known as protein kinase A (R. J. Lefkowitz, “G Protein Coupled Receptors III. New Roles for Receptor Kinases and β-Arrestins in Receptor Signaling and Desensitization,” J. Biol. Chem. 273: 18677-18680 (1998)). In this context, phosphorylation occurs on serine residues located in the third cytoplasmic loop or carboxyl-terminal tail of the receptors. Phosphorylation directly alters receptor conformation so that interaction of the receptor with the corresponding G protein is impaired. This type of receptor regulation generally mediates a type of desensitization referred to as heterologous or non-agonist-specific desensitization because any stimulant that elevates cAMP has the potential to cause the phosphorylation and resulting desensitization of any GPCR containing an appropriate protein kinase A (PKA) consensus phosphorylation site.

The major cellular mechanism mediating rapid, agonist-specific, or homologous desensitization of G protein-coupled receptors consists of a two-step process in which the agonist-occupied receptors are phosphorylated by a GRK and then bind an arrestin protein, which sterically interdicts signaling to the G protein. GRK-catalyzed activity may be allosteric; other factors regulating activity of GRKs include protein kinase C, lipids, and calcium-binding proteins such as recoverin or calmodulin. GRK phosphorylation of GPCRs potentiates the binding of arrestins and the internalization and sequestration of these receptors. Therefore, as stated below, any agents that can interfere with any aspect of this process may be useful for inhibiting the activity of arrestins and thus prevent desensitization of the β-adrenergic receptors.

One alternative of an agent that can inhibit the activity of arrestins is an inhibitor of the arrestin itself. Such inhibitors can block arrestin-mediated signaling. Inhibitors of arrestin, specifically arrestin-2, are described below.

One inhibitor of arrestin-2 activity is barbadin. Barbadin is an arrestin-2/β₂-adaptin interaction inhibitor that blocks agonist-promoted endocytosis of β₂-adrenergic receptors (A. Beautrait et al., “A New Inhibitor of the β-Arrestin/AP2 Endocytic Complex Reveals Interplay Between GPCR Internalization and Signalling,” Nature Commun. 8: 15054 (2017)).

Another inhibitor of arrestin-2 activity is inositol hexaphosphate (IP6). Inositol hexaphosphate is described as an arrestin sequestrant (Y. K. Peterson & L. M. Luttrell, “The Diverse Roles of Arrestin Scaffolds in G Protein-Coupled Receptor Signaling,” Pharmacol. Rev. 69: 256-297 (2017)).

Another inhibitor of arrestin-2 activity is ML339 (N-[(1S,5R)-9-[2-(2-chloroanilino)-2-oxoethyl]-9-azabicyclo[3.3.1]nonan-3-yl]-3,4,5-trimethoxybenzamide), which antagonizes arrestin-2 recruitment.

Another alternative of an agent that can inhibit the activity of arrestins is an inhibitor of GRKs. As used herein, the term “inhibitor of GRKs,” unless otherwise limited, refers to a compound that can inhibit the activity of at least one GRK; there is no requirement that such a compound inhibit the activity of all GRKs. Inhibitors of GRKs are disclosed in United States Patent Application Publication No. 2004/0053852 by Stamler et al. GRKs phosphorylate agonist-occupied GPCRs, thereby promoting binding by the β-arrestin molecules to the phosphorylated GPCRs. Therefore, agents that inhibit the activity of GRKs can also block the activity of β-arrestin. These agents include NO donors that donate nitric oxide or a related species, including S-nitroso, 0-nitroso, C-nitroso, and N-nitroso compounds. These compounds include, but are not limited to, S-nitrosoglutathione (GSNO), S-nitroso-N-acetylpenicillamine, S-nitroso-cysteine and ethyl ester thereof, S-nitroso-cysteinyl glycine, S-nitroso-γ-methyl-L-homocysteine, S-nitroso-L-homocysteine, S-nitroso-γ-thio-L-leucine, S-nitroso-δ-thio-L-leucine, and S-nitrosoalbumin. Examples of other NO donors useful herein are sodium nitroprusside (nipride), ethyl nitrite, nitroglycerin, SIN1(molsidomine), furoxamines, and N-hydroxy-(N-nitrosamine). Additional GRK inhibitors include, but are not limited to, paroxetine and Cmpd101 (3-[(4-methyl-5-pyridin-4-yl-1,2,4-triazol-3-yl)methylamino]-N-[[2-(trifluoromethyl)phenyl]methyl]benzamide) (S. M. Sulon & J. L. Benovic, “Targeting G Protein-Coupled Receptor Kinases (GRKs) to G Protein-Coupled Receptors,” Curr. Opin. Endocr. Metab. Res. 16: 56-65 (2021)).

Additionally, according to R. J. Lefkowitz, “G Protein Coupled Receptors III. New Roles for Receptor Kinases and β-Arrestins in Receptor Signaling and Desensitization,” J. Biol. Chem. 273: 18677-18680 (1998), protein kinase A inhibitors may also be useful in blocking the activity of arrestin.

Protein kinase A inhibitors include the following agents: (1) H89 (N-[2-[[3-(4-bromophenyl)-2-propenyl]amino]ethyl]-5-isoquinolinesulfonamide dihydrochloride) (J. Leemhuis et al., “The Protein Kinase A Inhibitor H89 Acts on Cell Morphology by Inhibiting Rho Kinase,” J. Pharmacol. Exp. Ther. 300: 1000-1007 (2002); (2) N-(ω-undecylenoyl)phenylalanine (U.S. Pat. No. 7,871,635 to Stolz et al.); (3) 3′,5′-cyclic monophosphorothioate-R, H-7 (5-(2-methylpiperazin-1-yl)sulfonylisoquinoline dihydrochloride), H-8 (N-[2-(methylamino)ethyl]-5-isoquinolinesulfonamide dihydrochloride), and H-9 (N-(2-aminoethyl)-5-isoquinolinesulfonamide) (U.S. Pat. No. 9,744,332 to Berggren et al.); (4) 6-22 amide, a peptide with the sequence Thr-Tyr-Ala-Asp-Phe-Ile-Ala-Ser-Gly-Arg-Thr-Gly-Arg-Arg-Asn-Ala-Ile-NH₂ (SEQ ID NO: 2) (U.S. Pat. No. 10,485,845 to Ambron et al.); protein kinase A inhibitors including fasudil, N-[2-(phosphorylated bromonitroarginylamino)ethyl]-5-isoquinoline sulfonamide, 1-(5-quinolinesulfonyl)piperazine, 4-cyano-3-methylisoquinoline, acetamido-4-cyano-3-methylisoquinoline, 8-bromo-2-monoacyladenosine-3,5-cyclic monophosphorothioate, adenosine 3,5-cyclic monophosphorothioate, 2-O-monobutyl-cyclic adenosine monophosphate, 8-chloro-cyclic adenosine monophosphate, N-[2-(cinnamoylamino acid)]-5-isoquinolinone, reverse phase-8-hexylamino adenosine 3,5-monophosphorothioate, reverse phase-8-piperidinyladenosine-cyclic adenosine monophosphate, reverse phase-adenosine 3,5-cyclic monophosphorothioate, 5-iodotuberculin, 8-hydroxyadenosine-3,5-monophosphorothioate, calphostin C, daphnetin, reverse phase-8-chlorophenyl-cyclic adenosine monophosphate, reverse phase-cyclic adenosine monophosphate, reverse phase-8-Br-cyclic adenosine monophosphate, 1-(5-isoquinolinesulfonyl)-2-methylpiperidine, 8-hydroxy adenosine-3′,5′-monophosphate, 8-hexylaminoadenosine-3′,5′-monophosphate, and reverse phase-adenosine 3′,5′-cyclic monophosphate (United States Patent Application Publication No. 2019/0343861 by Dai); erbstatin (United States Patent Application Publication No. 2008/0138834 by Emans et al.); protein kinase A inhibitors including adenosine 3′,5′-cyclic phosphorothiolate, 8-bromo-adenosine 3′,5′-cyclic monophosphorothioate, 4-cyano-3-methylisoquinoline, 1-(5-isoquinolinesulfonyl)-2-methylpiperazine, N-[2-(methylamino)ethyl]-5-isoquinolinesulfonamide, isoquinolinesulfonamide, N-2-aminoethyl)-5-isoquinolinesulfonamide, N-[²-((p-bromocinnamyl)amino)ethyl]-5-isoquinolinesulfonamide and (5-isoquinolinesulfonyl)piperazine (United States Patent Application Publication No. 2006/0099568 by Jang et al.); KT 5720 ((9R,10S,12S)-2,3,9,10,11,12-hexahydro-10-hydroxy-9-methyl-1-oxo-9,12-epoxy-1H-diindolo[1,2,3-fg:3′,2′,1′-kl]pyrrolo[3,4-i][1,6]benzodiazocine-10-carboxylic acid hexyl ester); myristoylated PKI 14-22 amide (Myr-Gly-Arg-Thr-Gly-Arg-Arg-Asn-Ala-Ile-NH₂); cAMPs-Rp triethylammonium salt ((R)-adenosine, cyclic 3′,5′-(hydrogenphosphorothioate) triethylammonium); daphnetin; HA-100 (5-(1-piperazinylsulfonyl)isoquinoline dihydrochloride); HA-100 (5-(1-piperazinylsulfonyl)isoquinoline dihydrochloride); staurosporine; and A-3 hydrochloride (N-(2-aminoethyl)-5-chloronaphthalene-1-sulfonamide hydrochloride).

In addition, as shown in Example 6, the enzyme phospholipase C plays a key role in the pathway leading to asthmatic symptoms, as it cleaves a phosphodiester bond in membrane phospholipids, resulting in the formation of a 1,2-diglyceride. Arachidonate is then released from the diglyceride by the sequential actions of diglyceride lipase and monoglyceride lipase. Once released, a portion of the arachidonate is metabolized rapidly, leading to oxygenated products, including eicosanoids such as prostaglandins. Thus, any treatment that can inhibit phospholipase C activity, including inhibitors of phospholipase C itself, is relevant for the treatment of diseases and conditions affecting the respiratory tract including infection with SARS-CoV-2 virus and its sequelae.

Therefore, in yet another alternative according to the present invention, the nadolol, the derivative or analogue of nadolol, or the prodrug of the nadolol or the derivative or analogue of the nadolol is administered together with a therapeutically effective quantity of a phospholipase C inhibitor.

Phospholipase C inhibitors include, but are not limited to: sodium aristolochate; D609 (sodium tricyclodecan-9-yl xanthogenate); D-erythro-dihydrosphingosine; U-73122 (1-(6-((17β-3-methoxyestra-1,3,5(10)-trien-17-yl)amino)hexyl)-1H-pyrrole-2,5-dione); pyrrolidinethiocarbamate; neomycin sulfate; thielavin B; edelfosine; compounds described in U.S. Pat. No. 7,262,197 to Lagu et al., United States Patent Application Publication No. 2004/0242639 by Lagu et al., United States Patent Application Publication No. 2004/0235855 by Lagu et al., and United States Patent Application Publication No. 2004/0235827 by Lagu et al., including heterocyclyl-substituted anilino compounds, including: N-[2-[4-(diphenylmethyl)-1-piperazinyl]-5-(1-piperazinylcarbonyl)phenyl]-4-methyl-benzamide; 5-(4-chlorophenyl)-N-[2-[4-(diphenylmethyl)-1-piperazinyl]-5-(1-piperazinylcarbonyl)phenyl]-2-methyl-3-furancarboxamide; N-[2-[4-(diphenylmethyl)-1-piperazinyl]-5-(1-piperazinylcarbonyl)phenyl]-2-furancarboxamide; N-[2-[4-(diphenylmethyl)-1-piperazinyl]-5-(1-piperazinylcarbonyl)phenyl]-propanamide; 3-[[(phenylamino)carbonyl]amino]-4-[4 -(phenylmethyl)-1-piperidinyl]-benzamide; 3-[[(phenylamino)carbonyl]amino]-4-(4-phenyl-1-piperidinyl)-benzamide; 3-[[(1,3-benzodioxol-5-ylamino)carbonyl]amino]-4-(4-phenyl-1-piperidinyl)-benzamide; N-[2-(4,4-diphenyl-1-piperidinyl)-5-(1-piperazinylcarbonyl)phenyl]-N′-phenyl-urea; N-[5-(aminocarbonyl)-2-[4-(phenylmethyl)-1-piperidinyl]phenyl]hydrazine-carboxamide; 4-[4-(diphenylmethyl)-1-piperidinyl]-3-[[(phenylamino)carbonyl]amino]-benzamide; 4-[4-(diphenylmethylene)-1-piperidinyl]-3-[[(phenylanino)carbonyl]amino]-benzamide; N-[2-[4-(diphenylmethyl)-1-piperidinyl]-4-(1-piperazinylcarbonyl)phenyl]-N′-phenyl-urea; N-cyclohexyl-N-[2-[4-(diphenylmethyl)-1-piperidinyl]-4-(1-piperazinylcarbonyl) phenyl]-urea; 4-[4-(2-methoxyphenyl)-1-piperazinyl]-3-[[(phenylamino)carbonyl]amino]-benzamide; 3-[[(phenylamino)carbonyl]amino]-4-[4-(phenylmethyl)-1-piperazinyl]-benzamide; 4-[4-[bis(4-fluorophenyl)methyl]-1-piperazinyl]-3-[[(phenylamino)carbonyl]amino]-benzamide; 4-[4-[bis(4-fluorophenyl)methyl]-1-piperazinyl]-3-[[[(2-fluorophenyl)amino]carbonyl]amino]-benzamide; 4-[4-(diphenylmethyl)-1-piperazinyl]-3-[[[(4-nitrophenyl)amino]carbonyl]amino]-benzamide; 4-[4-(diphenylmethyl)-1-piperazinyl]-3-[[[(phenylmethyl)amino]carbonyl]amino]-benzamide, 3-[[[(3,5-dimethylphenyl)amino]carbonyl]amino]-4-[4-(diphenylmethyl)-1-piperazinyl]-benzamide; 4-[4-(diphenylmethyl)-1-piperazinyl]-3-[[(phenylamino)carbonyl]amino]-benzamide; 4-[4-(9H-fluoren-9-yl)-1-piperazinyl]-3-[[(phenylamino)carbonyl]amino]-benzamide; 3-[[(cyclohexylamino)carbonyl]amino]-4-[4-(diphenylmethyl)-1-piperazinyl]-benzamide; 4-[4-(diphenylmethyl)-1-piperazinyl]-3-[[[[(1S)-1-phenylethyl]amino]carbonyl]amino]-benzamide; 3-[[(butylamino)carbonyl]amino]-4-[4-(diphenylmethyl)-1-piperazinyl]-benzamide; 4-[4-(diphenylmethyl)-1-piperazinyl]-3-[[[(4-fluorophenyl)amino]carbonyl]amino]-benzamide; 3-[[(1,3-benzodioxol-5-ylamino)carbonyl]amino]-4-[4-[bis-(4-fluorophenyl)methyl]-1-piperazinyl]-benzamide; 4-[4-[bis(4-fluorophenyl)methyl]-1-piperazinyl]-3-[[[(2,4-dimethylphenyl)amino]carbonyl]amino]-benzamide; 4-[4-[bis(4-fluorophenyl)methyl]-1-piperazinyl]-3-[[[(1-phenylethyl)amino]carbonyl]amino]-benzamide; 4-[4-[bis(4-fluorophenyl)methyl]-1-piperazinyl]-3-[[[(2-methoxyphenyl)amino]carbonyl]amino]-benzamide; 4-[4-[bis(4-fluorophenyl)methyl]-1-piperazinyl]-3-[[[(2,4-dimethoxyphenyl)amino]carbonyl]amino]-benzamide; 4-[4-[bis(4-fluorophenyl)methyl]-1-piperazinyl]-3-[[[[4-(dimethylamino)phenyl]amino]carbonyl]amino]-benzamide; 4-[4-[bis(4-fluorophenyl)methyl]-1-piperazinyl]-3-[[[(4-methoxyphenyl)amino]carbonyl]amino]-benzamide; 4-[4-[bis(4 -fluorophenyl)methyl]-1-piperazinyl]-3-[[[(phenylmethyl)amino]thioxomethyl]amino]-benzamide; 4-[4-[bis(4-fluorophenyl)methyl]-1-piperazinyl]-3-[[(phenylamino)thioxomethyl]amino]-benzamide; N-[2-[4-(diphenylmethyl)-1-piperazinyl]-5-[(4-methyl-1-piperazinyl)carbonyl]phenyl]-N′-phenylurea; N-[2-[4-(diphenylmethyl)-1-piperazinyl]-5-[(hexahydro-1H-1,4-diazepin-1-yl)carbonyl]phenyl]-N′-phenylurea, N-cyclohexyl-N′-[2-[4-(diphenylmethyl)-1-piperazinyl]-5-[(hexahydro-1H-1,4-diazepin-1-yl)carbonyl]-phenyl]urea; N-(2-aminoethyl)-4-[4-(diphenylmethyl)-1-piperazinyl]-3-[[(phenylamino)carbonyl]amino]-benzamide; and N-(2-aminoethyl)-3-[[(cyclohexylamino)carbonyl]amino]-4-[4-(diphenylmethyl)-1-piperazinyl]-benzamide; N-[2-[4-(diphenylmethyl)-1-piperazinyl]-5-(1-piperazinylcarbonyl)phenyl]-4-methyl-benzamide; 5-(4-chlorophenyl)-N-[2-[4-(diphenylmethyl)-1-piperazinyl]-5-(1-piperazinylcarbonyl)phenyl]-2-methyl-3-furancarboxamide; N-[2-[4-(diphenylmethyl)-1-piperazinyl]-5-(1-piperazinylcarbonyl)phenyl]-2-furancarboxamide; N-[2-[4-(diphenylmethyl)-1-piperazinyl]-5-(1-piperazinylcarbonyl)phenyl]-propanamide; N-[2-[4-(diphenylmethyl)-1-piperazinyl]-5-(1-piperazinylcarbonyl)phenyl]-4-methyl-benzenesulfonamide; 4-chloro-N-[2-[4-(diphenylmethyl)-1-piperazinyl]-5-(1-piperazinylcarbonyl)phenyl]-benzenesulfonamide; N-[2-[4-(diphenylmethyl)-1-piperazinyl]-5-(1-piperazinylcarbonyl)phenyl]-1-butanesulfonamide; N-[2-[4-(diphenylmethyl)-1-piperazinyl]-5-(1-piperazinylcarbonyl)phenyl]-methanesulfonamide; N-[2-[4-(diphenylmethyl)-1-piperazinyl]-5-(methylsulfonyl)phenyl]-N-phenyl-urea; N-[2-[4-[bis(4-fluorophenyl)methyl]-1-piperazinyl]-5-(hydroxymethyl)phenyl]-N-phenyl-urea; and 4-[4-[bis(4-fluorophenyl)methyl]-1-piperazin-yl]-3-[[(phenylamino)carbonyl]amino]-benzoic acid methyl ester; DCIC (3,4-dichloroisocoumarin); and calporoside or calporoside derivatives, including compounds disclosed in U.S. Pat. No. 6,596,984 to Eder et al., including compounds of Formula (C-I):

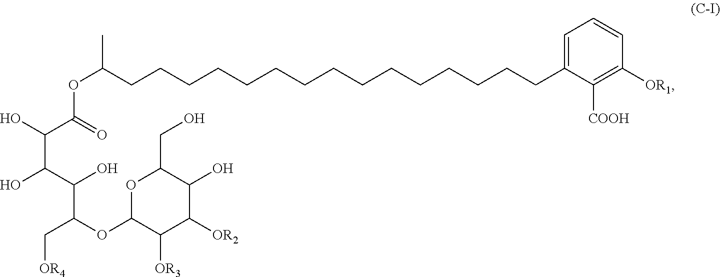

wherein:
(1) R₁, R₂, and R₃ are independently selected from the group consisting of H and C₁-C₁₀ acyl; and
(2) R₄ is H or —C(O)(CH₂)ₙCOOH, wherein n is from 1 to 7, with the proviso that R₁, R₂, R₃, and R₄ are not all H; RHC-80267 (1,6-bis-(cyclohexyloximinocarbonylamino)-hexane); o-phenanthroline; 2-morpholino-N-hydroxybenzamides (S. W. P. Rees et al., “Development of 2-Morpholino-N-hydroxybenzamides as Anti-Proliferative PC-PLC Inhibitors,” Bioorg. Chem. 114: 105152 (2021)); derivatives of benzenesulfonamides, pyrido[3,4-b]indoles, morpholinobenzoic acids, and benzamidobenzoic acids (C. Eurtivong et al., “Discovery of Novel Phosphatidylcholine-Specific Phospholipase C Drug-Like Inhibitors as Potential Anticancer Agents, Eur. J. Med. Chem. 187: 111919 (2020)); and derivatives of compounds including morpholino and 2-substituted N-benzyl moieties (L. I. Pilkington, “Development, Synthesis and Biological Investigation of a Novel Class of Potent PC-PLC Inhibitors,” Eur. J. Med. Chem. 191: 112162 (2020)).

In another alternative, the additional agent used in a method according to the present invention can be an agent for treating SARS-CoV-2 infection. Such agents can be used together with nadolol or a derivative or analogue of nadolol. Agents for treating SARS-CoV-2 infection include the following agents. These agents include both monoclonal antibodies and small molecules.

Monoclonal antibodies include both: (i) monoclonal antibodies that block the infection of susceptible cells by the SARS-CoV-2 virus, typically by binding to an epitope present in the spike protein of the virus; and (ii) monoclonal antibodies that act by other mechanisms.

Monoclonal antibodies that block the infection of susceptible cells by the SARS-CoV-2 virus by binding to an epitope present in the spike protein of the virus include, but are not necessarily limited to, bebtelovimab, bamlanivimab, etesevimab, casirivimab, imdemivab, tixagevimab, cilgavimab, regdanvimab, and sotrovimab. Monoclonal antibodies that act by other mechanisms include, but are not necessarily limited to, vilobelimab, sarilumab, lenzilumab, and levilimab.

Bebtelovimab is a human IgG1 monoclonal antibody typically administered intravenously in a dosage of 175 mg and that binds to the spike protein of SARS-CoV-2. Administration of bebtelovimab should be initiated within seven days of confirmed infection.

Bamlanivimab/etesevimab is a combination of two monoclonal antibodies, bamlanivimab and etesevimab, administered together via intravenous infusion as a treatment for SARS-CoV-2 infection. Bamlanivimab is an IgG1κ human monoclonal antibody directed against the spike protein of SARS-CoV-2. The aim is to block viral attachment and entry of the virus into human cells, preventing its replication. Etesevimab is a fully human recombinant monoclonal antibody directed against the spike protein of SARS-CoV-2. However, this combination of monoclonal antibodies is not likely to be effective against infections caused by the omicron variant of SARS-CoV-2, but may retain effectiveness against other variants or subvariants derived from other variants.

Casirivimab is a humanized monoclonal antibody. Imdevimab is also a humanized monoclonal antibody. These antibodies are typically administered in combination. However, this combination of monoclonal antibodies is not likely to be effective against infections caused by the omicron variant of SARS-CoV-2, but may retain effectiveness against other variants or subvariants derived from other variants.

Tixagevimab is an extended half-life recombinant monoclonal IgG1κ monoclonal antibody. Cilgavimab is also an extended half-life recombinant monoclonal IgG1κ monoclonal antibody. These monoclonal antibodies are also typically administered in combination. However, this combination of monoclonal antibodies is also considered not likely to be effective against infections caused by the omicron variant of SARS-CoV-2, but may retain effectiveness against other variants or subvariants derived from other variants.

Regdanvimab is a human monoclonal antibody directed against the spike protein of SARS-CoV-2.

Sotrovimab is a human monoclonal antibody also directed against the spike protein of SARS-CoV-2. As with a number of the monoclonal antibodies described above, this monoclonal antibody may lack effectiveness against some of the omicron subvariants. For example, for sotrovimab, in vitro activity is retained against the original omicron variant and against the omicron BA.1 and BA.1.1 subvariants, but the in vitro activity against the omicron BA.2 subvariant was shown to be substantially diminished.

Because these monoclonal antibodies are directed against a single epitope in the spike protein of SARS-CoV-2 virus, the efficacy of these monoclonal antibodies can be diminished by mutations in the spike protein, which can occur in variants. In particular, mutations in the spike protein occurring in the omicron variant and some of its subvariants have significantly diminished the efficacy of some of these monoclonal antibodies against the omicron variant and at least some of these subvariants.

The monoclonal antibody vilobelimab is a human-mouse chimeric IgG4κ monoclonal antibody that targets human C5a in plasma. It specifically binds to the soluble human complement split product C5a after cleavage from C5 to block its interaction with the C5a receptor, both of which are components of the complement system thought to contribute to inflammation and worsening of SARS-CoV-2 infection.

The monoclonal antibody sarilumab is a human monoclonal antibody against the interleukin-6 receptor and acts to block inflammation.

Lenzilumab is a humanized monoclonal antibody of class IgG1κ that targets colony stimulating factor 2 (CSF2)/granulocyte-macrophage colony stimulating factor (GM-CSF) and also acts to block inflammation.

Levilimab is a human IgG1 anti-IL6 receptor monoclonal antibody that has been proposed as a treatment of SARS-CoV-2; it appears to function by reducing inflammation caused by infection with SARS-CoV-2.

Additional agents for treating SARS-CoV-2 infection are small molecules. Some of these agents act by directly inhibiting the replication of SARS-CoV-2 virus. These agents include, but are not limited to: a fixed-dose combination of nirmatrelvir and ritonavir (marketed as a single pill as Paxlovid® for oral administration); remdesivir; and molnupiravir. Other agents act by different mechanisms as described below.

In the combination Paxlovid® oral medication, nirmatrelvir acts as an orally active 3C-like protease inhibitor. Coronaviral proteases cleave multiple sites in the viral polyprotein, most commonly after glutamine residues. Nirmatrelvir is a covalent inhibitor that binds to the catalytic cysteine (Cys145) residue of the cysteine protease. Ritonavir, in the combination Paxlovid® oral medication, acts as a booster of the protease activity of nirmatrelvir; more specifically, ritonavir is used to inhibit a particular enzyme present in the intestines, the liver, and elsewhere in the body, that normally metabolizes protease inhibitors; the enzyme is cytochrome P450-3A4 (CYP3A4). This enzyme catalyzes hydroxylation of its substrates; in some cases, hydroxylation is followed by dehydrogenation. A large range of additional inhibitors of CYP3A4 are known, including, but not limited to, boceprevir, indinavir, nelfinavir, saquinavir, clarithromycin, telithromycin, ceritinib, mibefradil, nefazodone, ribociclib, tucatinib, chloramphenicol, ketoconazole, itraconazole, posaconazole, voriconazole, and cobicistat. These agents could potentially be used together with Paxlovid®; in particular, the protease inhibitors boceprevir, indinavir, nelfinavir, and saquinavir could potentially be used together with Paxlovid® to further boost the protease activity of nirmatrelvir.

Preferably, Paxlovid® is administered within five days of the onset of symptoms in patients infected with SARS-CoV-2 virus. Administration of Paxlovid® is indicated in patients who are at high risk for progression to severe infection with SARS-CoV-2 virus, including the risk of hospitalization or death.

Remdesivir is a protide (prodrug of a nucleotide) that is able to diffuse into cells, where it is converted to GS-441524 (2R,3R,4S,5R)-2-(4-aminopyrrolo[2,1-f][1,2,4]triazin-7-yl)-3,4-dihydroxy-5-(hydroxymethyl)oxolane-2-carbonitrile) monophosphate via the action of esterases CES1 and CTSA and the phosphoamidase HINTl; the resulting monophosphate is then phosphorylated to the triphosphate, which is the active metabolite, through the action of nucleoside-phosphate kinase. This active metabolite interferes with the action of viral RNA-dependent RNA polymerase and also evades proofreading by viral exoribonuclease; the active metabolite acts as a delayed chain terminator of viral replication. Remdesivir is at least partially metabolized by the cytochrome P450 enzymes CYP2C8, CYP2D6, and CYP3A4.

Remdesivir is typically administered to patients infected with SARS-CoV2 virus in which the infection is severe enough to require hospitalization or supplemental oxygen; it can also be administered to patients who are not hospitalized, have a mild to moderate infection with SARS-Cov-2 virus, and are at high risk for progression to a severe infection, including hospitalization or death.

Favipravir is an antiviral agent that is a pyrazinecarboxamide derivative. It is activated by the hypoxanthine guanine phosphoribosyltransferase enzyme. It acts by inhibiting the replication of the SARS-CoV-2 virus.

Molnupiravir inhibits viral replication by promoting widespread mutations in the replication of viral RNA by RNA-directed RNA polymerase. It is metabolized into a ribonucleoside analogue that resembles cytidine, β-D-N⁴-hydroxycytidine-5′-triphosphate. During replication, the virus incorporates this analogue into newly synthesized RNA rather than cytidine. The active metabolite of molnupiravir has two tautomeric forms, one of which mimics cytidine and the other of which mimics uridine. The active metabolite of molnupiravir is not recognized as an error by the proofreading exonuclease enzymes of the virus; these enzymes can replace mutated nucleotides with corrected versions. The action of the active metabolite of molnupiravir is referred to as lethal mutagenesis. Molnupiravir is indicated for the treatment of mild to moderate SARS-CoV-2 infection who are at high risk for progression to severe infection.

T-1105 (2-oxo-1H-pyrazine-3-carboxamide) is an antiviral agent that is also activated by the hypoxanthine guanine phosphoribosyltransferase enzyme.

T-1106 (4-[(2R,3R,4S,5R)-3,4-dihydroxy-5-(hydroxymethyl)oxolan-2-yl]-3-oxopyrazine-2-carboxamide) is also an antiviral agent that is also activated by the hypoxanthine guanine phosphoribosyltransferase enzyme.

Lufotrelvir is an antiviral drug that acts as a 3CL protease inhibitor. It is a prodrug containing a phosphate moiety that is cleaved in vivo to yield the active agent PF-00835231 (N-[(2S)-1-[[(2S)-4-hydroxy-3-oxo-1-[(3S)-2-oxopyrrolidin-3-yl]butan-2-yl]amino]-4-methyl-1-oxopentan-2-yl]-4-methoxy-1H-indole-2-carboxamide).

Merafloxacin is a fluoroquinolone agent that inhibits the pseudoknot formation which is necessary for the frameshift occurring in translation of the SARS-CoV-2 virus genome (P. R. Bhatt et al., “Structural Basis of Ribosomal Frameshifting During Translation of the SARS-CoV-2 RNA Genome,” Science 372: 1306-1313 (2021)).

Olgotrevir is an antiviral medication that is believed to work by inhibiting the SARS-CoV-2 main protease (M^{pro}), which is a key enzyme required for replication of the virus, and also by blocking viral entry. The drug is a prodrug that is converted in vivo to its active form, AC1115 (2-[(E)-2-(4-methoxyphenyl)ethenyl]-4,6-bis(trichloromethyl)-1,3,5-triazine).

ASC-09 ([(3aS,4R,6aR)-2,3,3a,4,5,6a-hexahydrofuro[2,3-b]furan-4-yl] N-[(2S,3R)-4-[[2-[(1-cyclopentylpiperidin-4-yl)amino]-1,3-benzothiazol-6-yl]sulfonyl-(2-methylpropyl)amino]-3-hydroxy-1-phenylbutan-2-yl]carbamate) is a protease inhibitor that blocks the activity of a protease required for the replication of the virus.

Yet another class of small molecule agents that are anti-SARS-CoV-2 agents are SGLT2 inhibitors. SGLT2 is a member of the sodium glucose cotransporter family, and SGLT2 inhibitors have anti-inflammatory activity (B. M. Bonura et al., “Extraglycemic Effect of SGLT2 Inhibitors: A Review of the Evidence,” Diabetes Metab. Synd. Obesity: Targ. Ther. 13: 161-174 (2020)). SGLT2 inhibitors include, but are not limited to, canagliflozin, dapagliflozin, empagliflozin, ertugliflozin, ipragliflozin, luseogliflozin, remogliflozin etabonate, sergliflozin etabonate, sotagliflozin, and tofogliflozin. A particularly preferred SGLT2 inhibitor is dapagliflozin. Dapagliflozin reversibly inhibits sodium-glucose co-transporter 2 (SGLT2) in the renal proximal convoluted tubule to reduce glucose reabsorption and increase urinary glucose excretion.

Apabetalone is an agent that targets bromodomain and extra terminal domain (BET) proteins, and in particular the BET family member BRD4. BET proteins, which contain two bromodomains, interact with acetylated lysines on histones bound to DNA to regulate gene transcription via an epigenetic mechanism. Apabetalone selectively binds to the second bromodomain (BD2). When apabetalone binds to BRD4, it acts as an inhibitor of inflammation.

Still another class of small molecule agents that are anti-SARS-CoV-2 agents are agents that are inhibitors of the serine protease enzyme TMPRSS2 (transmembrane protease, serine 2). Some variants of SARS-CoV-2 virus are activated by the TMPRSS2 enzyme and thus can be blocked by TMPRSS2 inhibitors. Specifically, cleavage of the SARS-CoV-2 virus S2 spike protein required for viral entry into the cells can be catalyzed by the TMPRSS2 enzyme. Inhibitors of the TMPRSS2 enzyme include, but are not limited to, camostat, nafamostat, bromhexine (T. Shapira et al., “A TMPRSS2 Inhibitor Acts as a Pan-SARS-CoV-2 Prophylactic and Therapeutic,” Nature 605: 340-348 (2022)), and N-0385 ((2S)—N-[(2S)-1-[[(2S)-1-(1,3-benzothiazol-2-yl)-5-(diaminomethylideneamino)-1-oxopentan-2-yl]amino]-1-oxo-3-phenylpropan-2-yl]-2-(methanesulfonamido)pentanediamide). A particularly preferred inhibitor of TMPRSS2 is camostat, which has been shown to significantly reduce the infection of lung cells by SARS-CoV-2 and also to decrease C-reactive protein (CRP) levels, which is a marker of inflammation.

Another small molecule agent is danoprevir. Danoprevir is a macrocyclic peptidomimetic inhibitor of NS3/4A HCV protease. It contains acylsulfonamide, fluoroisoindole and tert-butyl carbamate moieties. It can block SARS-CoV-2 viral replication.

Yet another small molecule agent is deuremidevir. Deuremidevir is a nucleoside analogue antiviral drug that is a deuterated tri-isobutyrate of the active metabolite of remdesivir.

Still another small molecule agent is disulfiram. Disulfiram inhibits the papain-like protease of SARS-CoV-2 virus and may be useful for treatment of SARS-CoV-2 infection.

Yet another small molecule agent is ensitrelvir. Ensitrelvir is an antiviral medication that is a 3CL protease inhibitor (J. D. Tindall, “S-217622, a 3CL Protease Inhibitor and Clinical Candidate for SARS-CoV-2,” Med. Chem. 65: 6496-6498 (2022)).

Still another small molecule agent is simnotrelvir. Simnotrelvir is an inhibitor of the SARS-CoV-2 protease 3CL^{pro}, and is typically administered in combination with ritonavir.

Yet another small molecule agent is talfirastide. Talfirastide is a synthetic analogue of angiotensin 1-7 that works as an angiotensin II type I receptor-biased ligand. It has been tested in people with SARS-CoV-2 infection. It is believed to block the occurrence of an inflammatory state associated with the reduction of angiotensin associated with entry of the SARS-CoV-2 virus through the ACE-2 enzyme (angiotensin-converting enzyme 2), which renders the ACE-2 enzyme non-functional (C. Wagener et al., “A Randomized, Placebo-Controlled Double-Blinded Pilot Study of Angiotensin 1-7 (TXA-127) for the Treatment of Severe COVID-19,” Critical Care 26: 229 (2022)).

Still another small molecule agent is tipiracil. Tipiracil, typically administered as the hydrochloride, is a thymidine phosphorylase inhibitor and inhibits degradation of trifluridine. It has been shown to inhibit SARS-CoV-2 Nsp15 and interacts with the uridine binding pocket of the enzyme.

Yet another class of small molecule agents that are anti-SARS-CoV-2 agents are agents that blocks the activity of human protein eEF1A, a translation elongation factor that is involved in replication of viral proteins in infected cells. Agents that block the human protein eEFLA include, but are not necessarily limited to, plitidepsin (K. M. White et al., “Plitidepsin Has Potent Preclinical Efficacy Against SARS-CoV-2 by Targeting the Host Protein eEF1A,” Science 371: 926-931 (2021)).

Still another class of small molecule agents that are anti-SARS-CoV-2 agents are antidepressants. Antidepressants considered to potentially have activity against SARS-CoV-2 virus include, but are not limited to, the selective serotonin reuptake inhibitors fluoxetine and fluvoxamine.

Still another class of agents that are anti-SARS-CoV-2 agents are anti-mitotic agents that act to depolymerize microtubules and prevent their polymerization. These anti-mitotic agents act to inhibit the transport of viral particles, including those of SARS-CoV-2 virus. Anti-mitotic agents include, but are not limited to, cabazitaxel, docetaxel, epothilone, ixabepilone, larotaxel, neoxaline, ortataxel, paclitaxel, sabizabulin, tesetaxel, demecolcine, vinblastine, nocodazole, vincristine, vindesine, vinflunine, and vinorelbine. A particularly preferred anti-mitotic agent is sabizabulin.

Yet another class of agents that are anti-SARS-CoV-2 virus agents include vaccines that prevent or reduce the replication of the SARS-CoV-2 virus. Although, in general, such vaccines do not completely block initial infection with the virus, they typically reduce the severity and shorten the course of infection in the event that an infection occurs. The vaccines originally in use were developed against the original strain of the SARS-CoV-2 virus, and their effectiveness varies considerably against more recent variants and subvariants that have emerged, particularly the omicron variant and subvariants thereof. However, particularly with respect to mRNA vaccines, as described below, new versions of these vaccines have been developed. These new versions of the vaccines are intended to provide protection against either the omicron variants or several of its subvariants that are in circulation. Additionally, immunity induced by the vaccines currently in use tends to wane after a period of several months, after which a booster may be required to restore immunity. The period during which immunity remains sufficient to either block an infection or lessen its course or severity varies with the immunological capacity of the immunized patient and with the particular variant or subvariant that attempts to infect the immunized patient. However, although the β-adrenergic inverse agonists such as nadolol or derivatives or analogues of nadolol do not directly block the replication of the SARS-Cov-2 virus, they are useful in conjunction with vaccination with the vaccines. Additionally, their effectiveness does not depend on the particular variant or subvariant of the virus that infects a patient.

Vaccines authorized for use or proposed for use against SARS-CoV-2 infection include: mRNA vaccines, which typically contain mRNA encoding the SARS-Cov-2 spike protein; adenovirus-based vaccines, which use an adenovirus shell containing DNA that encodes a SARS-Cov-2 protein; inactivated vaccines that consist of virus particles that are grown in culture and then inactivated using a method such as heat and formaldehyde to lose disease-producing capacity while still inducing an immune response; subunit vaccines, which present one or more antigens without introducing entire virions; virus-like particle vaccines; DNA plasmid vaccines; lentivirus vector vaccines; conjugate vaccines; and vesicular stomatitis virus vaccines displaying the SARS-CoV-2 virus spike protein. In general, these vaccines are administered by injection, typically via the intramuscular route. Additionally, immunity induced by these vaccines typically takes some time to build up; the generally-accepted period during which maximum immunity develops is two weeks for mRNA vaccines. Accordingly, one of ordinary skill in the art can readily determine the optimum time and period of administration of the nadolol or derivative or analogue of nadolol in immunized patients.

In methods and compositions according to the present invention, for small molecules other than nadolol or derivatives or analogues of nadolol as described above useful in combination with the β-adrenergic inverse agonist can be optionally substituted with one or more groups that do not substantially affect the pharmacological activity of the small molecule. Definitions for a number of common groups that can be used as optional substituents have been provided above; however, the omission of any group from these definitions cannot be taken to mean that such a group cannot be used as an optional substituent as long as the chemical and pharmacological requirements for an optional substituent are satisfied.

As used herein, the term “alkyl” refers to an unbranched, branched, or cyclic saturated hydrocarbyl residue, or a combination thereof, of from 1 to 12 carbon atoms that can be optionally substituted; the alkyl residues contain only C and H when unsubstituted. Typically, the unbranched or branched saturated hydrocarbyl residue is from 1 to 6 carbon atoms, which is referred to herein as “lower alkyl.” When the alkyl residue is cyclic and includes a ring, it is understood that the hydrocarbyl residue includes at least three carbon atoms, which is the minimum number to form a ring. As used herein, the term “alkenyl” refers to an unbranched, branched or cyclic hydrocarbyl residue having one or more carbon-carbon double bonds. As used herein, the term “alkynyl” refers to an unbranched, branched, or cyclic hydrocarbyl residue having one or more carbon-carbon triple bonds; the residue can also include one or more double bonds. With respect to the use of “alkenyl” or “alkynyl,” the presence of multiple double bonds cannot produce an aromatic ring. As used herein, the terms “hydroxyalkyl,” “hydroxyalkenyl,” and “hydroxyalkynyl,” respectively, refer to an alkyl, alkenyl, or alkynyl group including one or more hydroxyl groups as substituents; as detailed below, further substituents can be optionally included. Further potential substituents for specific compounds have been described above.

Substituent groups useful for substituting saturated carbon atoms in the specified group, moiety, or radical include, but are not limited to, —Z^{a}, ═O, —OZ^{b}, —SZ^{b}, ═S⁻, —NZ^{c}Z^{c}, ═NZ^{b}, ═N—OZ^{b}, trihalomethyl, —CF₃, —CN, —OCN, —SCN, —NO, —NO₂, ═N₂, —N₃, — S(O)₂Z^{b}, —S(O)₂NZ^{b}, —S(O₂)O⁻, —S(O₂)OZ^{b}, —OS(O₂)OZ^{b}, —OS(O₂)O⁻, —OS(O₂)OZ^{b}, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)Z^{b}, —NZ^{b}C(S)Z^{b}, —NZ^{b}C(O)O⁻, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, —NZ^{b}C(NZ^{b})NZ^{c}Z^{c}, wherein Z^{a} is selected from the group consisting of alkyl, cycloalkyl, heteroalkyl, cycloheteroalkyl, aryl, arylalkyl, heteroaryl and heteroarylalkyl; each Z^{b} is independently hydrogen or Z^{a}; and each Z is independently Z^{b} or, alternatively, the two Z^{c}'s may be taken together with the nitrogen atom to which they are bonded to form a 4-, 5-, 6-, or 7-membered cycloheteroalkyl ring structure which may optionally include from 1 to 4 of the same or different heteroatoms selected from the group consisting of N, O, and S. As specific examples, —NZ^{c}Z^{c} is meant to include —NH₂, —NH-alkyl, —N-pyrrolidinyl, and —N-morpholinyl, but is not limited to those specific alternatives and includes other alternatives known in the art. Similarly, as another specific example, a substituted alkyl is meant to include -alkylene-O-alkyl, -alkylene-heteroaryl, -alkylene-cycloheteroaryl, -alkylene-C(O)OZ^{b}, -alkylene-C(O)NZ^{b}Z^{b}, and —CH₂—CH₂—C(O)—CH₃, but is not limited to those specific alternatives and includes other alternatives known in the art. The one or more substituent groups, together with the atoms to which they are bonded, may form a cyclic ring, including, but not limited to, cycloalkyl and cycloheteroalkyl.

Similarly, substituent groups useful for substituting unsaturated carbon atoms in the specified group, moiety, or radical include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —N₃, —S(O)₂Z^{b}, —S(O₂)O⁻, —S(O₂)OZ^{b}, —OS(O₂)OZ^{b}, —OS(O₂)O⁻, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and NZ^{b}C(NZ^{b})NZ^{c}Z^{c}, wherein Z^{a}, Z^{b}, and Z^{c} are as defined above.

Similarly, substituent groups useful for substituting nitrogen atoms in heteroalkyl and cycloheteroalkyl groups include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —S(O)₂Z^{b}, —S(O₂)O⁻, —S(O₂)OZ^{b}, —OS(O₂)OZ^{b}, —OS(O₂)O⁻, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)OZ^{b}, —C(S)OZ^{b}, —C(O)NZ^{c}Z^{c}, —C(NZ^{b})NZ^{c}Z^{c}, OC(O)Z^{b}, —OC(S)Z^{b}, —OC(O)OZ^{b}, —OC(S)OZ^{b}, —NZ^{b}C(O)Z^{b}, —NZ^{b}C(S)Z^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and NZ^{b}C(NZ^{b})NZ^{c}Z^{c}, wherein Z^{a}, Z^{b}, and Z^{c} are as defined above.

The compounds described herein may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms (such as the enantiomerically pure isomers, the E and Z isomers, and other stereoisomeric forms) as well as mixtures of stereoisomers in varying degrees of chiral purity or percentage of E and Z, including racemic mixtures, mixtures of diastereomers, and mixtures of E and Z isomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. Such separation techniques include, but are not limited to, chiral high pressure liquid chromatography (HPLC) and formation and crystallization of chiral salts. The invention includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the invention to the depicted olefin isomer. When the chemical name does not specify the isomeric form of the compound, it denotes any one of the possible isomeric forms or mixtures of those isomeric forms of the compound.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein includes two or more interconvertible compounds resulting from at least one formal migration of a hydrogen atom and at least one change in valency (e.g., a single bond to a double bond, a triple bond to a double bond, a triple bond to a single bond, or vice versa). The exact ratio of the tautomers present can depend on several factors, including pH, solvent, and temperature. Tautomerization reactions can be catalyzed by acid or base. Examples of tautomerization include keto/enol, amide/imide, lactam/lactim, and enamine/imine.

In addition to the substituents described above, alkyl, alkenyl and alkynyl groups can alternatively or in addition be substituted by C₁-C₈ acyl, C₂-C₈ heteroacyl, C₆-C₁₀ aryl, C₃-C₈ cycloalkyl, C₃-C₈ heterocyclyl, or C₅-C₁₀ heteroaryl, each of which can be optionally substituted. Also, in addition, when two groups capable of forming a ring having 5 to 8 ring members are present on the same or adjacent atoms, the two groups can optionally be taken together with the atom or atoms in the substituent groups to which they are attached to form such a ring.

“Heteroalkyl,” “heteroalkenyl,” and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. For reasons of chemical stability, it is also understood that, unless otherwise specified, such groups do not include more than two contiguous heteroatoms except where an oxo group is present on N or S as in a nitro or sulfonyl group.

Similarly, “heterocyclyl” may be used to describe a non-aromatic cyclic group that contains at least one heteroatom (typically selected from N, O and S) as a ring member and that is connected to the molecule via a ring atom, which may be C (carbon-linked) or N (nitrogen-linked); and “heterocyclylalkyl” may be used to describe such a group that is connected to another molecule through a linker. The heterocyclyl can be fully saturated or partially saturated, but non-aromatic. The sizes and substituents that are suitable for the cycloalkyl, cycloalkylalkyl, heterocyclyl, and heterocyclylalkyl groups are the same as those described above for alkyl groups. The heterocyclyl groups typically contain 1, 2 or 3 heteroatoms, selected from N, O and S as ring members; and the N or S can be substituted with the groups commonly found on these atoms in heterocyclic systems. As used herein, these terms also include rings that contain a double bond or two, as long as the ring that is attached is not aromatic. The substituted cycloalkyl and heterocyclyl groups also include cycloalkyl or heterocyclic rings fused to an aromatic ring or heteroaromatic ring, provided the point of attachment of the group is to the cycloalkyl or heterocyclyl ring rather than to the aromatic/heteroaromatic ring.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a Cr-arylalkyl group, and phenylethyl is a C₈-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. The general term “alkylene” encompasses more specific examples such as “ethylene,” wherein n is 2, “propylene,” wherein n is 3, and “butylene,” wherein n is 4. The hydrocarbyl groups of the alkylene can be optionally substituted as described above.

In general, any alkyl, alkenyl, alkynyl, acyl, or aryl or arylalkyl group that is contained in a substituent may itself optionally be substituted by additional substituents. The nature of these substituents is similar to those recited with regard to the primary substituents themselves if the substituents are not otherwise described.

“Amino” as used herein refers to —NH₂, but where an amino is described as “substituted” or “optionally substituted,” the term includes NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

Other combinations of substituents are known in the art and are described, for example, in U.S. Pat. No. 8,344,162 to Jung et al. For example, the term “thiocarbonyl” and combinations of substituents including “thiocarbonyl” include a carbonyl group in which a double-bonded sulfur replaces the normal double-bonded oxygen in the group. The term “alkylidene” and similar terminology refer to an alkyl group, alkenyl group, alkynyl group, or cycloalkyl group, as specified, that has two hydrogen atoms removed from a single carbon atom so that the group is double-bonded to the remainder of the structure.

Accordingly, methods and compositions according to the present invention encompass analogues and derivatives of small molecules, other than nadolol or the analogues or derivatives of nadolol described above, that are optionally substituted, provided that the optionally substituted small molecules possess substantially equivalent pharmacological activity to the unsubstituted small molecules as defined in terms of their activity. The activity can be assayed by methods known in the art, including enzyme assays, in vivo assays on airway hyperresponsiveness, assays of the effect of the optionally substituted small molecules on arrestin-2 concentration or activity, assays determining the effect of the optionally substituted small molecules on the activity of β₂-adrenergic receptors, and other methods known in the art. Suitable methods can be selected by one of ordinary skill in the art depending on the activity of the small molecule involved. Such optionally substituted small molecules include, but are not necessarily limited to, molecules in which the substitutions are considered to be bioisosteric. Bioisosterism is a well-known tool for predicting the biological activity of compounds, based on the premise that compounds with similar size, shape, and electron density can have similar biological activity. To form a bioisostere of a given molecule, one can replace one or more atoms or groups in the original molecule with known bioisosteric replacements for that atom or group. Known bioisosteric replacements include, but are not necessarily limited to, the interchangeability of —F, —OH, —NH₂, —Cl, and —CH₃, the interchangeability of —Br and -i-C₃H₇; the interchangeability of —I and -t-C₄H₉; the interchangeability of —O—, —S—, —NH₂—, —CH₂—, and —Se—; the interchangeability of —N═, —CH═, and —P═ in cyclic or noncyclic moieties; the interchangeability of phenyl and pyridyl groups; the interchangeability of —C═C— and —S(for example, benzene and thiophene); the interchangeability of an aromatic nitrogen (R₁—N(R₃)—R₂) for an unsaturated carbon ((R₁—C(═R₃)—R₂); and the interchangeability of —CO—, —SO—, and —SO₂—. Other alternatives for bioisosteric replacements are known in the art.

When a therapeutically active compound employed in methods or compositions according to the present application is a protein, protein fragment, polypeptide, or peptide, the protein, protein fragment, polypeptide, or peptide can be modified by the inclusion of one or more conservative amino acid substitutions, as long such conservative amino acid substitutions substantially preserve the biological activity of the therapeutically active compound. More specifically, in a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al., Molecular Biology of the Gene, 4th Edition, 1987, Benjamin/Cummings, p. 224). In particular, such a conservative variant has a modified amino acid sequence, such that the change(s) do not substantially alter the protein's (the conservative variant's) secondary or tertiary structure and/or activity, specifically binding activity in this context. Conservative amino acid substitution generally involves substitutions of amino acids with residues having similar properties (e.g., acidic, basic, positively or negatively charged, polar or non-polar, or other similarities) such that the substitutions of even critical amino acids do not substantially alter structure and/or activity. Conservative substitution tables providing functionally similar amino acids are well known in the art. For example, one exemplary guideline to select conservative substitutions includes (original residue followed by exemplary substitution): Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu. An alternative exemplary guideline uses the following six groups, each containing amino acids that are conservative substitutions for one another: (1) alanine (A or Ala), serine (S or Ser), threonine (T or Thr); (2) aspartic acid (D or Asp), glutamic acid (E or Glu); (3) asparagine (N or Asn), glutamine (Q or Gln); (4) arginine (R or Arg), lysine (K or Lys); (5) isoleucine (I or Ile), leucine (L or Leu), methionine (M or Met), valine (V or Val); and (6) phenylalanine (F or Phe), tyrosine (Y or Tyr), tryptophan (W or Trp); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. As another example, for some purposes, one may regard all non-polar amino acids as conservative substitutions for each other.

Yet another aspect of the present invention is a pharmaceutical composition comprising:
(1) a therapeutically effective quantity of nadolol or a derivative or analogue of nadolol to inhibit the β-arrestin pathway to treat the infection with SARS-CoV-2 virus; and
(2) a pharmaceutically acceptable carrier.

Preferably, the composition comprises nadolol. However, in an alternative, the composition can comprise a derivative or analogue of nadolol or a prodrug of nadolol as described above.

Typically, administration of the pharmaceutical composition exerts a therapeutic effect that is a reduction in pulmonary airway constriction hyperresponsiveness. Also typically, administration of the pharmaceutical composition exerts a therapeutic effect that is an upregulation of pulmonary β₂-adrenergic receptors. Also typically, administration of the pharmaceutical composition exerts a therapeutic effect that is increased pulmonary airway relaxation responsiveness to β₂-adrenergic agonist drugs.

Typically, the pharmaceutical composition is formulated for administration by a route selected from the group consisting of oral, sustained-release oral, parenteral, sublingual, buccal, administration by insufflation, and administration by inhalation. Preferably, the pharmaceutical composition is formulated for administration by the sustained-release oral route, particularly when the pharmaceutical composition comprises nadolol. However, in some alternatives described below, when the pharmaceutical composition comprises an additional therapeutic agent for the treatment of infection by SARS-CoV-2 virus for which the optimum route of administration is other than oral or sustained-release oral administration, such as parenteral administration. Unless further limited, the term “parenteral administration” refers to one or more of intravenous administration, intramuscular administration, subcutaneous administration, and intradermal administration. Typically, however, when a pharmaceutical composition according to the present invention includes an additional agent or agents in addition to the nadolol or derivative or analogue of nadolol, and an additional agent included in the pharmaceutical composition is a protein or peptide such as, but not limited to, an antibody, an antibody fragment, or a fragment of arrestin-2, the pharmaceutical composition is formulated for parenteral administration or administration by inhalation or insufflation in order to avoid the action of proteolytic enzymes in the digestive tract and thus ensure greater bioavailability of the protein or peptide.

In one alternative, administration of the pharmaceutical composition results in continuous levels of the nadolol or the derivative or analogue of nadolol in the bloodstream, particularly when the composition is formulated for administration by the sustained-release oral route.

When the pharmaceutical composition comprises nadolol, in one alternative, the quantity of nadolol in the composition is selected from the group consisting of 1 mg, 3 mg, 5 mg, 10 mg, 15 mg, 30 mg, 50 mg, and 70 mg per unit dose. Alternatively, other unit dose quantities of nadolol can be used.

The composition can further comprise at least one additional therapeutic agent for the treatment of infection with SARS-CoV-2 virus or its sequelae, as described above with respect to methods for treatment of infection with SARS-CoV-2 virus or its sequelae.

In one alternative, the composition can further comprise a therapeutically effective quantity of a suitable β₂-selective adrenergic agonist as described above.

In another alternative, the composition can further comprise a therapeutically effective quantity of a suitable corticosteroid as described above.

In yet another alternative, the composition can further comprise a therapeutically effective quantity of a suitable biological as described above.

In still another alternative, the composition can further comprise a therapeutically effective quantity of a suitable anticholinergic drug as described above.

In yet another alternative, the composition can further comprise a therapeutically effective quantity of a suitable xanthine compound as described above.

In still another alternative, the composition can further comprise a therapeutically effective quantity of a suitable anti-IgE antibody as described above.

In yet another alternative, the composition can further comprise a therapeutically effective quantity of a suitable leukotriene antagonist as described above.

In still another alternative, the composition can further comprise a therapeutically effective quantity of a suitable phosphodiesterase IV inhibitor as described above.

In yet another alternative, the composition can further comprise a therapeutically effective quantity of a suitable 5-lipoxygenase inhibitor as described above.

In still another alternative, the composition can further comprise a therapeutically effective quantity of a suitable mast cell stabilizer as described above.

In yet another alternative, the composition can further comprise a therapeutically effective quantity of a suitable arrestin-2 inhibitor, such as, but not limited to: a protein fragment of arrestin-2; a compound of Formula (A-I) as described above; an omega-3 fatty acid as described above; a CXCR2 inhibitor as described above; a MyD88 inhibitor as described above; a MD2 inhibitor as described above; inositol hexaphosphate; barbadin; or MIL339 as described above.

In yet another alternative, the composition can further comprise a therapeutically effective quantity of a suitable inhibitor of a GRK as described above. Inhibitors of a GRK can act as indirect inhibitors of arrestin-2.

In still another alternative, the composition can further comprise a therapeutically effective quantity of a suitable inhibitor of protein kinase A as described above. Inhibitors of protein kinase A can act as indirect inhibitors of arrestin-2.

In yet another alternative, the composition can further comprise a therapeutically effective quantity of an agent that is a suitable anti-SARS-CoV-2 virus agent, wherein the anti-SARS-CoV-2 virus agent is an agent that directly interferes with virus replication as described above.

In still another alternative, the composition can further comprise a therapeutically effective quantity of an agent that is a suitable anti-SARS-CoV-2 virus agent, wherein the anti-SARS-CoV-2 virus agent is an agent that is a monoclonal antibody that specifically binds to an antigen of the SARS-CoV-2 virus as described above.

In yet another alternative, the anti-SARS-CoV-2 virus agent is a suitable inhibitor of the serine protease enzyme TMPRSS2 as described above.

In still another alternative, the anti-SARS-CoV-2 virus agent is a suitable SGLT2 inhibitor as described above.

In yet another alternative, the anti-SARS-CoV-2 virus agent is a suitable selective serotonin reuptake inhibitor as described above.

In still another alternative, the anti-SARS-CoV-2 virus agent is a suitable anti-mitotic agent that acts to depolymerize microtubules and prevent their polymerization as described above.

In additional alternatives of compositions according to the present invention comprising an additional therapeutic agent, the additional therapeutic agent can comprise: (i) an IL-6 inhibitor; (ii) an IL-1 inhibitor; (iii) a JAK pathway inhibitor; (iv) a TNF-α inhibitor; (v) an IL-17 inhibitor; (vi) an agent that inhibits MUC5AC; (vii) an EGFR tyrosine kinase inhibitor; (viii) a p38 mitogen-activated protein kinase inhibitor; (ix) an anti-IDO antibody, clone 10.1; (x) a statin; or (xi) BIO-11006 acetate as described above.

In compositions according to the present invention, typically, the pharmaceutically acceptable carrier is selected from the group consisting of a solvent, a dispersion medium, a coating, an antibacterial agent, an antifungal agent, an isotonic agent, an absorption delaying agent, a preservative, a sweetening agent for oral administration, a thickening agent, a buffer, a liquid carrier, a wetting, solubilizing, or emulsifying agent; an acidifying agent, an antioxidant, an alkalinizing agent, a carrying agent, a chelating agent, a colorant, a complexing agent, a suspending or viscosity-increasing agent, a flavor or perfume, an oil, a penetration enhancer, a polymer, a stiffening agent, a protein, a carbohydrate, a bulking agent, and a lubricating agent.

The invention is illustrated by the following Examples. These Examples are included for illustrative purposes only, and are not intended to limit the invention.

### EXAMPLES

### Example 1

### Airway Resistance Reduction by Chronic Administration of β-Adrenergic Inverse Agonists

### Methods

Balb/cJ mice aged 6 weeks (Jackson Animal Laboratory, Bar Harbor, Maine) were housed under specific pathogen-free conditions and fed a chicken ovalbumin-free diet. The effects of administration of the non-selective β-adrenergic inverse agonists carvedilol (GlaxoSmithKline, King of Prussia, PA) and nadolol (Sigma Chemical, St. Louis, MO) and of salbutamol (Sigma Chemical, St. Louis, MO), a β₂-adrenergic partial agonist, were examined in a murine model that exhibited cardinal features of human asthma, such as pulmonary eosinophilic inflammation, airway hyperresponsiveness, and heterogeneous airway narrowing. The results obtained in drug-treated animals were compared with those obtained in vehicle-treated counterparts (controls) in experiments performed in close temporal relationship. The outcome measures of the study of Example 1 included statistically-significant differences between drug-treated mice and non-treated animals in terms of baseline airway resistance, degree of airway responsiveness to cholinergic stimulation, and bronchoalveolar lavage (BALF) cellularity. Mice were sensitized with subcutaneous injection of 25 μg of ovalbumin adsorbed to aluminum hydroxide on protocol days 2, 9, and 16. Subsequently, mice were given 50 μL of saline solution containing 25 μg of ovalbumin intranasally, on a daily basis, from protocol days 23 through 27. A group of ovalbumin-sensitized saline-challenged mice serves as controls for systemic sensitization and respiratory challenges with ovalbumin. Prior to intranasal administrations, mice were sedated with halothane vapor. For the study of Example 1, ovalbumin-sensitized and ovalbumin-challenged mice, and ovalbumin-sensitized and saline-challenged mice will be referred to as asthmatic mice and control mice, respectively. The drugs used were salbutamol (a β₁/β₂-adrenergic agonist), alprenolol (a β₁/β₂-adrenergic antagonist with partial agonist activity), and nadolol and carvedilol (both non-selective β₁/β₂ adrenergic inverse agonists).

To examine the effects of duration of β-adrenergic ligand therapy on the phenotype of the murine model of asthma, the experimental drugs were administered either acutely or chronically to different groups of asthmatic mice.

Asthmatic mice on acute therapy were given a single intravenous bolus infusion of either β-adrenergic drug or normal saline on protocol day 28, 15 minutes before airway responsiveness to methacholine was determined. The doses of carvedilol, nadolol, alprenolol, and salbutamol administered to the mice were 24 mg/kg, 72 mg/kg, 72 mg/kg, and 0.15 mg/kg, respectively. Asthmatic mice on chronic therapy were treated with the β-adrenergic drug during protocol days 1 to 28. Those on β-antagonists had free access to chow treated with carvedilol, nadolol, or alprenolol at concentrations of 2400 ppm, 250 ppm, or 7200 ppm, respectively. These concentrations were chosen based on those producing therapeutic effects in mice in previously published studies. The non-asthmatic mice were fed normal chow. Salbutamol was delivered for 28 days at a dose of 0.5 mg/kg/day using an osmotic minipump (Alzet®, #2004, Durect Corporation, Cupertino, CA).

On protocol day 28, mice were anesthetized, tracheotomized, and connected to a computer-controlled small animal ventilator (Flexivent, Scientific Respiratory Equipment, Inc., Montreal, Canada). Airway resistance (R_{aw}) was measured using the forced oscillation technique. The cellular composition of bronchoalveolar lavage fluid (BALF) was also determined. In non-treated asthmatic mice, the degree of airway responsiveness and the number of eosinophils recovered in BALF were significantly higher compared to the ovalbumin-sensitized saline-challenged (control) mice. However, it was observed that the degree of airway responsiveness and the number of eosinophils recovered in BALF were lower in non-treated asthmatic mice studied in close temporal relationship with mice receiving acute β-adrenergic antagonist treatments that in those obtained in non-treated asthmatic mice studied concomitantly with mice on chronic β-adrenergic antagonist therapy.

To induce airway constriction, a solution containing 150 μg/mL of acetyl-α-methylcholine chloride (methacholine) (Sigma Chemical, St. Louis, MO) was infused intravenously at constant rates using a syringe infusion pump (Raze Scientific Instruments, Stanford, CN). The methacholine infusion was started at 0.008 mL/min, and its rate was doubled stepwise up to a maximum of 0.136 mL/min. Each methacholine dose was administered for 3 to 5 minutes, during which data were sampled at 1-minute intervals and then averaged.

The complex input impedance of the respiratory system was computed and the value of the real part of respiratory system impedance at 19.75 Hz was taken to reflect the magnitude of airway resistance (R_{aw}). To examine the degree of airway responsiveness of each animal, the values for R_{aw} as a function of methacholine doses were plotted. The largest value for R_{aw} achieved in response to methacholine stimulation was referred to as R_{awpeak}. For mice that achieved a plateau in the methacholine dose-R_{aw} response curve, the ED₅₀ was calculated by linear interpolation using the GraphPad Prism4 (GraphPad Software, Inc.). Results obtained for β-adrenergic drug-treated and non-treated mice were performed using the analysis of variance for multiple groups of a Student's t-test for comparing two groups. The Bonferroni test was used to examine the statistical differences between experimental groups. The effects of acute drug treatments on baseline respiratory system mechanics were assessed using a two-tailed paired t-test. A value of P<0.05 was considered significant.

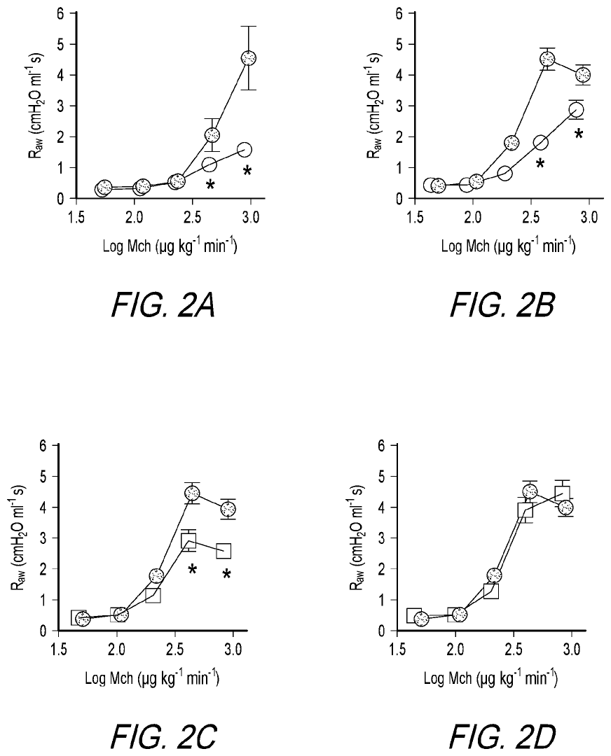

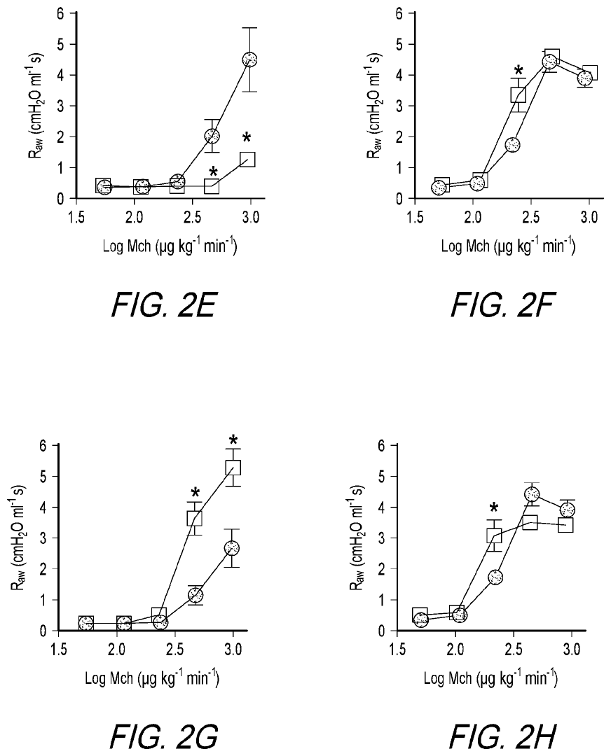

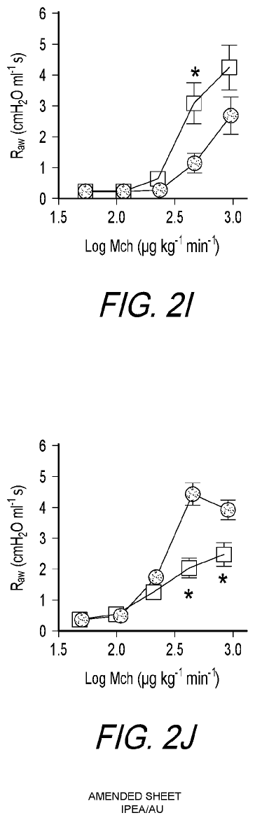

### FIG. 2

FIGS. 2A and 2B show that methacholine provocation significantly enhances airway resistance (R_{aw}) in asthmatic mice in contrast to a minimal response upon saline provocation of asthmatic mice. This demonstrates that the mouse model in this study exhibits airway hyperresponsiveness, a key feature of airway dysfunction in infection with SARS-CoV-2 virus or its sequelae.

In FIG. 2C, the administration of a single intravenous bolus of salbutamol to asthmatic mice reduced the level of airway responsiveness to methacholine provocation and the level of airway resistance as expected, thus demonstrating an acute effect of this agent. However, in FIG. 2D, when salbutamol was delivered for 28 days to the mice, no protection was observed. This lack of reduction of airway hyperresponsiveness upon chronic administration of a β-adrenergic agonist has been observed in humans when tolerance to these drugs develop. The issue of tolerance to such β-adrenergic agonists is also relevant with respect to the administration of these drugs for the treatment of infection with SARS-CoV-2 virus or its sequelae.

In FIG. 2E, when asthmatic mice were given a single intravenous bolus of alprenolol, a β-adrenergic antagonist with partial agonist activity, their airway responsiveness was diminished, as indicated by significant decreases in both the values for R_{aw} at methacholine doses≥408 μg/kg/min (P<0.05) compared with those obtained in non-treated counterparts. The reduction in airway responsiveness upon acute administration of alprenolol is similar to that observed for salbutamol, consistent with the partial agonist activity that alprenolol possesses. In FIG. 2F, when asthmatic mice were exposed to alprenolol for 28 days, their average methacholine dose-response relationship was similar to that obtained in nontreated mice, demonstrating that this drug provides no benefit upon chronic administration, as is the case with salbutamol. This is again directly analogous to the tolerance seen in human patients after long-term administration of such drugs, including when such drugs are administered for the treatment of infection by SARS-CoV-2 virus or its sequelae.

In FIG. 2G, a single intravenous bolus of carvedilol enhanced the airway responsiveness in the asthmatic mice. This is consistent with previous observations in humans that acute delivery of β-adrenergic antagonists to asthmatics can result in severe airway constriction. In contrast, in FIG. 2H, chronic administration of carvedilol reduced the responsiveness of asthmatic mice to methacholine provocation.

In FIG. 2I, a single intravenous bolus of nadolol also enhanced the airway responsiveness of asthmatic mice similar to the result observed for carvedilol. Chronic delivery of nadolol, as shown in FIG. 2J, also produced a decrease in airway responsiveness, which was more pronounced than that caused by carvedilol treatment. Indeed, the average methacholine dose-R_{aw} response relationship obtained in asthmatic mice on chronic nadolol treatment was similar to that obtained in mice on acute salbutamol treatment.

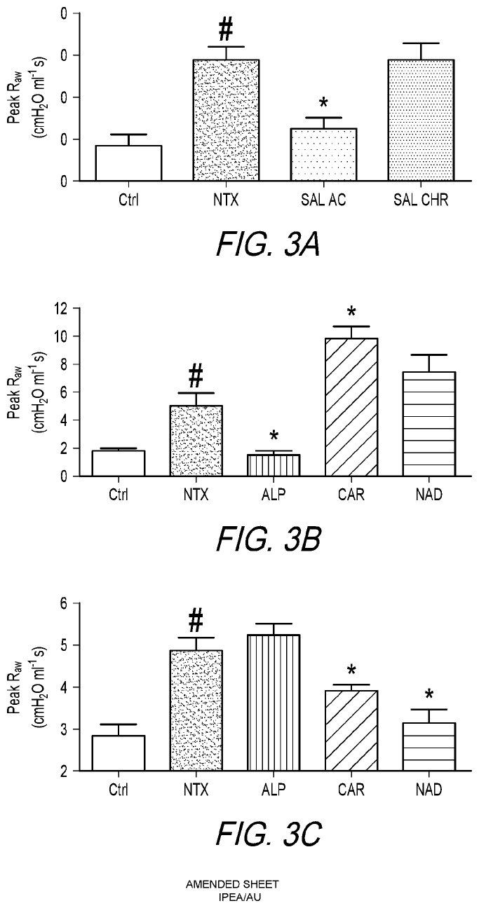

### FIG. 3

FIG. 3 shows the effects of administration of β-adrenergic receptor ligands on the peak airway responsiveness to cholinergic stimulation in asthmatic mice. Peak R_{aw} was determined for each mouse by examining the individual methacholine dose-response curves and choosing the highest R_{aw} value produced by any of the methacholine doses (most often the next to last dose, 408 μg kg⁻¹ min⁻¹). Shown are the mean peak R_{aw}±SEM after treatments with the β-adrenergic receptor agonist salbutamol (A), after acute treatments with various agents (B) (ALP=alprenolol; CAR=carvedilol; NAD; nadolol); and after chronic treatments with the same agents used in (B), all in comparison to nontreated asthmatic mice (NTX) (black bars, n=7-25) and control mice (Ctrl, white bars, n=6-21). Values are mean±SEM for the peak R_{aw} values to methacholine of n=8-19 mice. Note the change in scale of the y-axis for (B). *, P<0.05 compared to NTX; #, P<0.05 compared to Ctrl (ANOVA).

The results of Example 1 are applicable to infection by SARS-CoV-2 virus and its sequelae as the infection and its sequelae involve exacerbations of airway resistance and are associated with the consequences of hyperinflammation. The resulting symptoms associated with exacerbations of airway resistance can be severe, and can increase morbidity and mortality in patients infected by SARS-CoV-2 virus, particularly in elderly patients or patients with risk factors such as obesity or diabetes.

### Example 2

### Chronic Inverse Agonist Treatment Increases β-Adrenergic Receptor Numbers as Measured by Radioligand Binding

β₂-adrenergic receptor numbers were measured in asthmatic mice as follows. Asthmatic mice (ovalbumin-challenged) were treated as follows: Ctrl, no drug treatment with methacholine challenge; salbutamol, a short-acting R₂ agonist; carvedilol, a β₁, β₂, non-selective inverse agonist with ai-adrenergic antagonist activity; nadolol, a highly specific, hydrophilic β₁, β₂, non-selective inverse agonist; and alprenolol, a β-adrenergic antagonist. Drug treatments were either a single treatment 15 minutes prior to methacholine challenge or ongoing for 28 days (salbutamol was delivered continuously via a subcutaneous osmotic minipump and alprenolol, carvedilol, and nadolol were in animal chow). Mice were sacrificed and lung membranes were isolated as follows. Frozen lung tissue was homogenized in an ice-cold buffer containing 0.32 M sucrose and 25 mM Tris (pH 7.4) using a polytron (Pro 200, Pro Scientific, Inc.). The homogenate was centrifuged at 1000×g for 10 min at 4° C. The resulting supernatant was centrifuged at 40,000×g for 20 min at 4° C. The pellet was suspended in an ice-cold 25 mM Tris-HCl buffer (pH 7.4) and centrifuged at 40,000×g for 20 min at 4° C. The final pellet was suspended in 200 μL of 25 mM Tris-HCl (pH 7.4); membrane protein concentration was determined by BCA protein assay kit. Radioligand receptor binding incubation mixtures contained membranes (~10 μg of protein), (−)3-[¹²⁵I] cyanopindolol (ICYP) in 25 mM Tris-HCl, pH 7.4, in increasing concentrations (5-7500 pM) and binding buffer in a final volume of 250 μL. Propranolol was used to determine nonspecific binding. The incubation was done at 37° C. for 2 h and terminated by rapid vacuum filtration through glass fiber filters. The filters were washed three times with 250 μL of cold wash buffer (25 mM Tris-HCl, pH 7.4) and the radioactivity determined in a counter. All experiments were performed in triplicate and values are mean±SEM of n=3-5 animals in each group. Receptor densities are expressed as femtomoles of sites per milligram of protein. Bₘₐₓ is determined by nonlinear regression of the saturation binding curves. Apparent K_{D} values (in parentheses) are expressed as pM. Please note that the 15-minute and 28-day time points refer to duration of drug treatment. All mice were killed at the same age and thus for vehicle treated groups (CtrI and NTX) the groups were identical and the results pooled. #P<0.05 compared to Ctrl; *P<0.05 compared to NTX (Student's t-test).

Radioligand binding revealed that 2-adrenergic receptor levels appear to be somewhat lower in methacholine-challenged but otherwise untreated asthmatic mice as compared with untreated, unchallenged mice, as shown in Table 1. Chronic alprenolol treatment led to a slight decrease of the level of the β₂-adrenergic receptor. The same was true of chronic salbutamol treatment. Most significantly, the carvedilol-treated mice demonstrated an over 10 -fold increase of the level of β₂-adrenergic receptors over the non-treated mice, demonstrating the efficacy of this β-adrenergic inverse agonist in increasing receptor levels upon chronic administration. Similarly, the nadolol-treated mice demonstrated a nearly eightfold increase of the level of receptors over the untreated methacholine-challenged asthmatic mice.

TABLE 1
Determination of β-Adrenergic Receptor Density by Radioligand Binding

| | 15 Minutes | | 28 Days | |
|---|---|---|---|---|
| Treatment | Bₘₐₓ | K_{D} | Bₘₐₓ | K_{D} |

| | | |
|---|---|---|
| Ctrl | 286.8 ± 88.02 (107.9 ± 43.67) | 286.8 ± 88.02 (107.9 ± 43.67) |
| NTX | 109.2 ± 9.72 # (193.6 ± 20.66) | 109.2 ± 9.72 # (193.6 ± 20.66) |
| Salbutamol | 256.5 ± 29.24* (228.8 ± 33.07) | 97.0 ± 23.02 (225.4 ± 41.79) |
| Alprenolol | 299.5 ± 12.19* (453.6 ± 86.33) | 179.2 ± 53.05 (290.9 ± 55.07) |
| Carvedilol | 86.3 ± 19.42 (565.2 ± 192.8)* | 904.1 ± 43.46* (1444.0 ± 202.0) |
| Nadolol | 181.9 ± 48.28 (695.1 ± 286.3)* | 785.5 ± 154.8* (1591.6 ± 335.0)* |

The results of Example 2 are relevant to the treatment of infection by SARS-CoV-2 virus and its sequelae as the density of β₂-adrenergic receptors is important for response to agonists used for the treatment of respiratory diseases and conditions as described above, including, but not limited to, infection by SARS-CoV-2 virus and its sequelae, which, as stated above, are marked by airway hyperinflammation. As stated above, the resulting symptoms associated with airway hyperinflammation can be severe, and can increase morbidity and mortality in patients infected by SARS-CoV-2 virus, particularly in elderly patients or patients with risk factors such as obesity or diabetes.

### Example 3

### Chronic Inverse Agonist Treatment Increases β-Adrenergic Receptor Numbers as Monitored by Immunochemistry

For immunohistochemistry analysis of β₂-adrenergic receptor levels, non-drug-treated control mice and mice treated chronically with the β₂-adrenergic inverse agonist nadolol were used. The mice were sacrificed and the lungs excised. Then the lungs were fixed in 4% paraformaldehyde (45 min, 0° C.). After fixation, lungs were washed in PBS (60 min) and placed in increasing concentrations of sucrose (10% sucrose/5% glycine in PBS for 30 min; 20% sucrose/10% glycine in PBS for 30 min; 30% sucrose/15% glycine in PBS for 12 h at 4° C.). Lungs were embedded in OCT and 12-μm sections cut with a Tissue-Tek II cryostat. The sections were air-dried and fixed with 4% paraformaldehyde for 15 minutes. After 3 washes in PBS, the slides were blocked with 5% milk in PBS for 1 hour, and then incubated overnight with anti-β₂-adrenergic receptor antibody (1:200, Santa Cruz Biotechnology) in blocking solution. Slides were washed in PBS and incubated with secondary antibody (1:200, Cy3 goat anti-rabbit antibody, 16 h at 4° C.). Control slides were incubated with antibody specific blocking peptide to demonstrate specificity of binding of the primary antibody. After washing with PBS, coverslips were mounted and viewed by epifluorescent microscopy.

The results show that labeling with anti-R₂-adrenergic receptor antibodies was considerably more intense in lungs from animals treated with nadolol than in lungs from untreated animals. Loss of this signaling upon incubation in the presence of the β₂-adrenergic receptor peptide demonstrates that this antibody is specifically binding the β₂-adrenergic receptor. This observation is consistent with the radioligand binding data of Example 2 and suggests that β₂-adrenergic receptors are effectively upregulated by chronic administration of 32-adrenergic inverse agonist drugs.

These results suggest that administration of β₂-adrenergic inverse agonist drugs, particularly nadolol, can reduce airway resistance and hyperinflammation in patients infected with SARS-CoV-2 virus.

### Example 4

### Effect of Combination of Carvedilol and Salbutamol on Airway Hyperresponsiveness

The effect of combination therapy with carvedilol and salbutamol was compared to monotherapy with carvedilol alone on airway hyperresponsiveness in asthmatic mice.

Mice (Balb/cJ) aged 6 weeks were housed under specific pathogen-free conditions and fed a chicken ovalbumin-free diet. Mice were systemically sensitized with ovalbumin adsorbed to aluminum hydroxide. Mice were treated as follows: CAR/SAL 28D=for 28 days mice (n=6-12) were administered carvedilol (2400 ppm in animal chow) and salbutamol (subcutaneous delivery of 0.5 mg/kg/day in an Alzet #2400 osmotic minipump); NTX S/C=mice (n=6-12) no drug treatment for 28 days; CTRL=mice (n=6-12) no drug treatment for 28 days, not subsequently challenged; CARHD 28D=for 28 days mice (n=6-12) were administered carvedilol only (2400 ppm in animal chow); CARHD 28D SAL AC=for 28 days mice (n=6-12) were administered carvedilol (2400 ppm in animal chow) and 15 minutes prior to measuring airway hyperresponsiveness, salbutamol was administered at a dose of 1.2 mg/kg.

To measure airway hyperresponsiveness after 28 days, all mice except the CTRL (control) mice were challenged with ovalbumin and then all mice were anesthetized, tracheotomized, and connected to a Flexivent small animal ventilator to measure airway resistance (R_{aw}) by the forced oscillation technique. To induce airway constriction, a solution containing 150 μg/mL of methacholine was infused using a syringe infusion pump. The methacholine infusion was started at 0.008 mL/min and its rate was doubled stepwise up to a maximum 0.136 mL/min. Each methacholine dose was administered until a plateau was reached, during which data were sampled at 1-minute intervals for 3-5 minutes and then averaged.

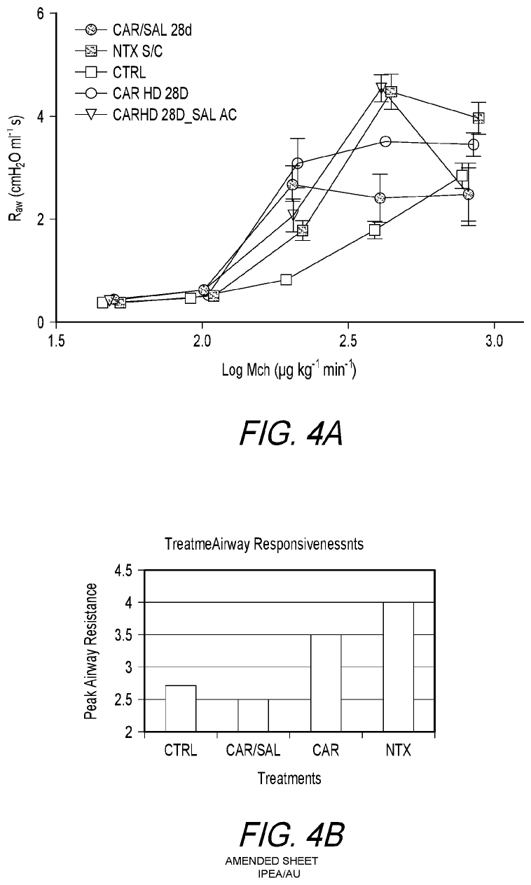

In FIG. 4A, at the highest dose of methacholine, both of the combination drug treatments were equally effective in preventing bronchoconstriction and not statistically significantly different from the control mice which were only challenged with saline solution. The carvedilol monotherapy resulted in higher bronchoconstriction than these treatments but less than the non-drug treated sensitized and challenged (NTX S/C) mice. Thus, the combination therapy of β₂-adrenergic inverse agonist and agonist with the agonist administered either chronically or acutely is effective at ameliorating airway hyperresponsiveness to allergen and methacholine challenge and is an improvement over the monotherapy of the β₂-adrenergic inverse agonist alone.

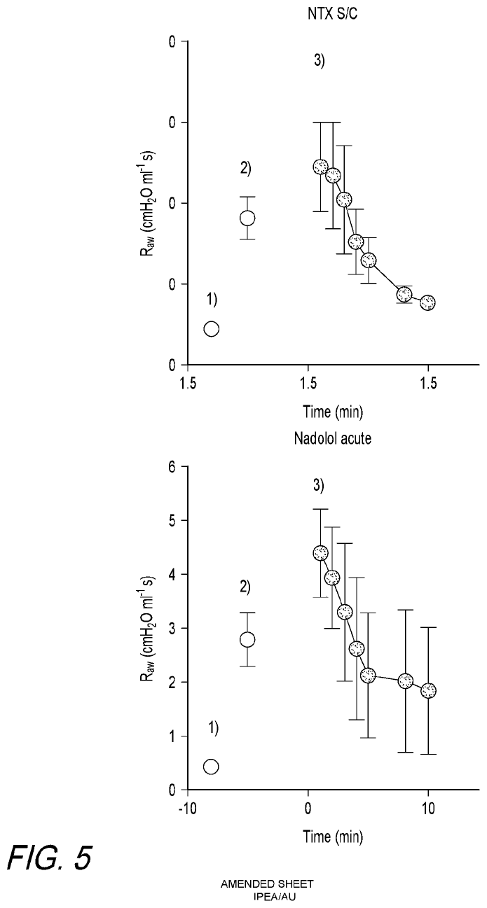

This data is summarized in FIG. 4B, which shows that the combination of carvedilol and salbutamol is the most effective in reducing airway hyperresponsiveness of the treatments for which the results are shown in FIG. 5A. This indicates the effectiveness of the use of combination therapy of β₂-adrenergic inverse agonist and agonist. These results would apply to treatment of infection by SARS-CoV-2 virus and its sequelae, which are characterized by abnormal airway hyperresponsiveness, particularly in elderly patients or patients characterized by one or more risk factors.

### Example 5

Effect of Combination Therapy with Aminophylline on Acute Airway Effects of Nadolol

Mice were sensitized to the allergen ovalbumin as described in Example 1. Mice were then challenged with allergen and then subjected to methacholine-induced bronchoconstriction challenge, non-drug treated, NTX S/C, or pretreated with nadolol at 0.72 mg/kg i.p. for 15 minutes prior to methacholine challenge (nadolol acute treatment).

At time point 1 (time=−10 min) baseline airway resistance of the mice was determined. At time point 2 (time=−5 min) methacholine was infused into mice to reach their EC₇₀. At time point 3 (time=0 min) aminophylline was administered i.p. at a dose of 100 mg/kg.

In FIG. 5, pretreatment of mice with nadolol resulted in the same baseline airway resistance as non-drug treated sensitized and allergen-challenged mice. However, upon methacholine challenge, the nadolol-treated mice exhibited a much higher airway resistance of ~4.5 versus 2.5 units. Upon administration of aminophylline, there was a significant and sustained drop in airway resistance in both the untreated and the nadolol-treated mice.

Z. Callaerts-Vegh et al., “Effects of Acute and Chronic Administration of β-Adrenoceptor Ligands on Airway Function in a Murine Model of Asthma,” Proc. Natl. Acad. Sci. USA 101: 4948-4953 (2004), have shown that while nadolol administered chronically prevents airway hyperresponsiveness in the same mouse asthma model, nadolol administered acutely worsens airway hyperresponsiveness. These data demonstrate that the addition of the methylxanthine aminophylline can alleviate the acute effects on airway hyperresponsiveness of nadolol administration. This is beneficial in that the opportunity exists for asthma subjects to take nadolol chronically to prevent bronchoconstriction. These subjects then can co-administer a methylxanthine such as aminophylline to prevent the acute detrimental effects of nadolol. These effects are temporary in duration, but can impair patient compliance with a therapeutic regimen. These effects are addressed for methods and compositions according to the present invention in order to improve patient compliance, in particular by adjusting the initial dosage of nadolol and the adjustment of the dosage during treatment in order to treat airway symptoms such as hyperinflammation and airway resistance associated with infection with SARS-CoV-2 virus and its sequelae.

### Example 6

Effect of Treatment with Salbutamol or Nadolol on the Ratio of Phospholipase C to Actin in Cultured Tracheal Smooth Muscle Cells

Cultured tracheal smooth muscle cells were obtained from mice exposed to the following treatments: NS/NC=nonasthmatic, non-challenged mice; S/C=asthmatic mice; Sal.Ac=asthmatic mice, acute salbutamol treatment; Sal.Ch=asthmatic mice, chronic salbutamol treatment; Nad.Ac=asthmatic mice, acute nadolol high dose treatment; and Nad.Ch=asthmatic mice, chronic nadolol high dose treatment.

After airway function experiments, the tracheas were surgically removed from anesthetized mice that had been treated with drugs or vehicle. The tracheas were minced and the cells plated and grown in culture. The smooth muscle cells grow faster and take over the culture dish. The cells were grown in medium which contained the drugs used in the treatment or vehicle controls. Phospholipase C (PLC-(β1) was determined by immunoblotting with an antibody specific for the enzyme. Actin was used as a loading control and the amount of PLC-β1 was expressed as a ratio of phospholipase C to actin.

The enzyme phospholipase C plays a key role in the pathway leading to asthmatic symptoms, as it cleaves a phosphodiester bond in membrane phospholipids, resulting in the formation of a 1,2-diglyceride. Arachidonate is then released from the diglyceride by the sequential actions of diglyceride lipase and monoglyceride lipase. Once released, a portion of the arachidonate is metabolized rapidly, leading to oxygenated products, including eicosanoids such as prostaglandins. Thus, any treatment that can inhibit phospholipase C activity is relevant for the treatment of asthma. Similarly, inhibitors of phospholipase C are also relevant for the treatment of other respiratory diseases and conditions such as infection with SARS-CoV-2 virus and its sequelae.

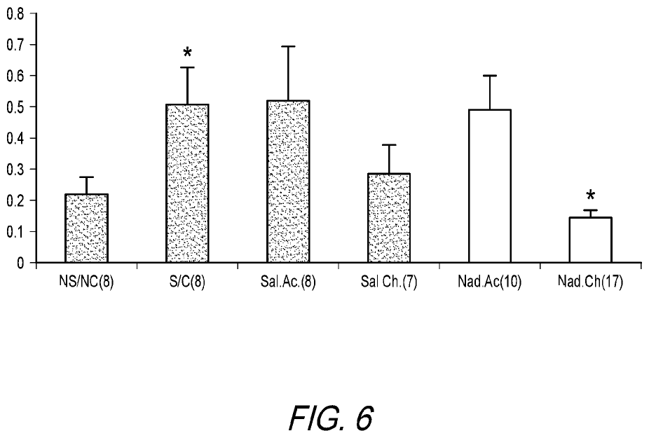

The results are shown in FIG. 6. The results shown in FIG. 6 indicate that chronic administration of nadolol significantly decreases the activity of phospholipase C. This indicates that such chronic administration of nadolol is effective against asthma, chronic obstructive pulmonary disease, and other diseases and conditions affecting the respiratory tract, including infection with SARS-CoV-2 virus and its sequelae, and associated with inflammation and airway hyperresponsiveness and prevents activation of some of the mechanisms that lead to the symptoms of asthma and other such conditions, including infection with SARS-CoV-2 virus and its sequelae.

### Example 7

### Effect of β-Adrenergic Receptor Drugs at Low and High Doses on Airway Resistance

For these experiments, salbutamol was used for chronic administration at 0.5 mg/kg/day with a minipump and for acute administration at 0.15 mg/kg by i.v. bolus 15 minutes prior to challenge. Alprenolol was used at a high dose of 7200 ppm in chow or at a low dose of 720 ppm in chow. Carvedilol was used at a high dose of 2400 ppm in chow or at a low dose of 720 ppm in chow. Nadolol was used at a high dose of 250 ppm in chow or at a low dose of 25 ppm in chow. Nadolol was also tested at 1 ppm in chow and these results were identical to the untreated mice.

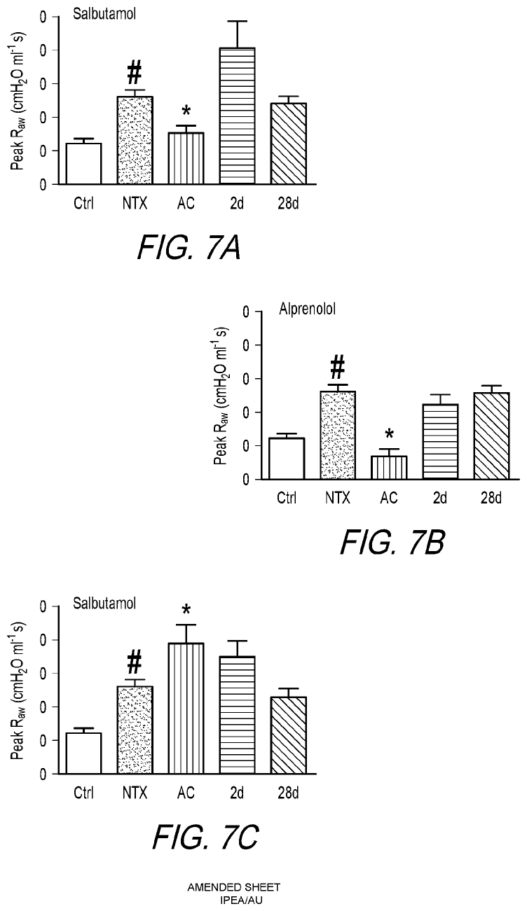

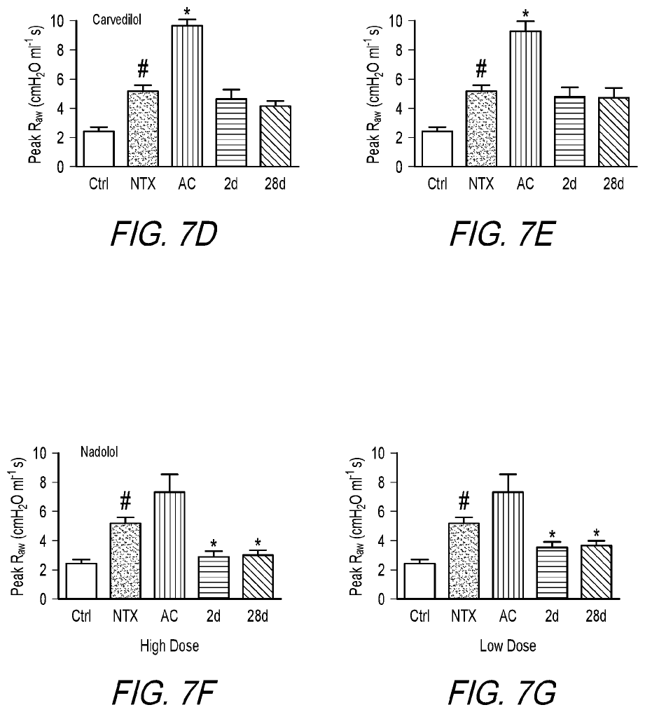

The results are shown in FIGS. 7A (salbutamol); 7B (high-dose alprenolol); 7C (low-dose alprenolol); 7D (high-dose carvedilol); 7E (low-dose carvedilol); 7F (high-dose nadolol); and 7G (low-dose nadolol). In these diagrams, Ctrl=control mice, non-asthmatic, non-drug treated; NTX=asthmatic mice, non-drug treated; AC=acute administration; 2d=chronic administration for 2 days; 28d=chronic administration for 28 days. The airway resistance (R_{aw}) is plotted as cm H₂O ml⁻¹ s. The data particularly shows the effect of the β-adrenergic inverse agonists carvedilol and nadolol in providing protection from airway hyperresponsiveness with chronic administration. These results are relevant with respect to the use of such β-adrenergic inverse agonists, particularly nadolol, in treating the consequences of infection with SARS-CoV-2 virus and thus preventing airway hyperinflammation, particularly in patients with risk factors.

### Example 8

Correlation of Decrease in Airway Resistance with Upregulation of β-Adrenergic Receptor Density

The correlation of the decrease in airway resistance with the upregulation of β-adrenergic receptor density for three different periods of administration of salbutamol, alprenolol, carvedilol, and nadolol is shown in Table 2. The periods of administration of the agents are 15 minutes, 2 days, and 28 days. Only the inverse agonists carvedilol and nadolol showed an increase in βadrenergic receptor density at periods longer than 15 minutes; carvedilol showed an increase in receptor density at 28 days, while nadolol showed an increase in receptor density at both 2 days and 28 days. There was an exact correlation between the decrease of airway resistance (R_{aw}) and the increase in receptor density. This strongly supports the concept of combination therapy, such as with an inverse agonist and an agonist, for the treatment of diseases and conditions such as infection with SARS-CoV-2 virus and its sequelae.

TABLE 2
Correlation of Decrease in Airway Resistance with Upregulation of β₂-Adrenergic Receptor Density

| | 15 minutes | | 2 days | | 28 days | |
|---|---|---|---|---|---|---|
| | | Increased | | Increased | | Increased |
| | Decreased R_{aw} | β₂AR density | Decreased R_{aw} | β₂AR density | Decreased R_{aw} | β₂AR density |
| Salbutamol | yes | yes | no | no | no | no |
| Alprenolol | yes | yes | no | no | no | no |
| Carvedilol | no | no | no | no | yes | yes |
| Nadolol | no | no | yes | yes | yes | yes |

### Example 9

Effect of Chronic Treatment with Metoprolol and Timolol on Airway Responsiveness in Asthmatic Mice

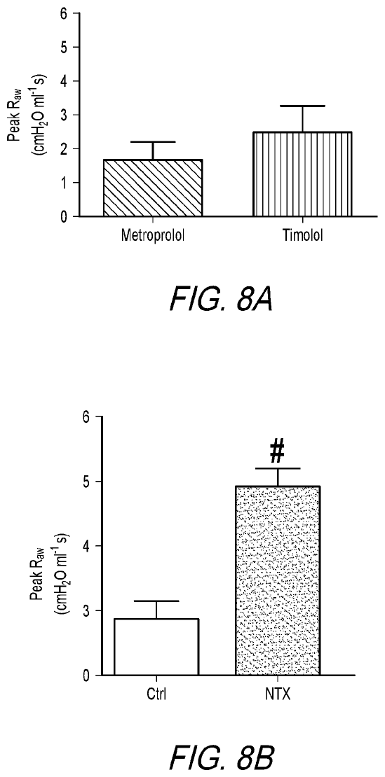

The protocols of Example 1 were followed for two additional inverse agonists, metoprolol (dosage of 20 mg/kg administered 3×daily via subcutaneous injection for 7 days) and timolol (dosage of 20 mg/kg in chow for 7 days), using asthmatic mice and methacholine challenge as in Example 1. Airway resistance (R_{aw}) was measured as in Example 1. The results for metoprolol and timolol are shown in FIG. 8A. The results were compared to historical controls as shown in FIG. 8B: Ctrl, no drug treatment, no challenge with methacholine; NTX, no drug treatment, challenged with methacholine. The results indicate that chronic treatment with both metoprolol and timolol are effective in reducing airway hyperresponsiveness in asthmatic mice, showing their utility for treatment of infection by SARS-CoV-2 virus and its sequelae. More importantly, these results support the general concept of using β-adrenergic inverse agonists for treatment of infection by SARS-CoV-2 virus and its sequelae.

### Example 10

### Administration of Nadolol Prevents Mucous Metaplasia

The occurrence of mucous metaplasia can lead to severe consequences in asthma and other airway diseases associated with chronic airway obstruction, particularly in infection by SARS-CoV-2 virus and its sequelae.

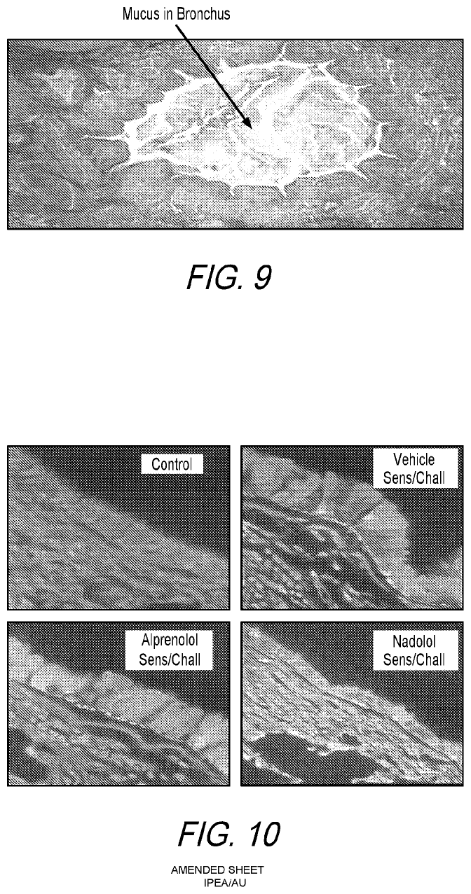

FIG. 9 is a photomicrograph showing the occurrence of a mucus plug in the bronchus of an 8-year-old girl with fatal asthma. FIG. 10 is a series of photomicrographs showing that nadolol is effective in preventing mucous metaplasia while the antagonist alprenolol is ineffective in preventing mucous metaplasia: top left, control; top right, sensitized/challenged mice without treatment showing mucous metaplasia; bottom left, sensitized/challenged mice after treatment with alprenolol showing no improvement in mucous metaplasia; bottom right, sensitized/challenged mice after treatment with nadolol showing nearly complete elimination of mucous metaplasia.

Therefore, nadolol is highly effective in eliminating or preventing mucous metaplasia which, in turn, can prevent serious consequences that may otherwise occur due to the accumulation of mucus in the respiratory tract. Such serious consequences are characteristic of infection by SARS-CoV-2 virus and its sequelae.

### Example 11

### Nadolol Reverses Epithelial Changes Via Inhibition of the Beta-Arrestin Pathway

Nadolol reverses epithelial changes via inhibition of the beta-arrestin pathway in β₂-adrenergic receptors, which, in turn, results in greater therapeutic efficacy when R₂-adrenergic agonists are administered to treat diseases and conditions affecting the respiratory tract, such as asthma, as well as infection by SARS-CoV-2 virus and its sequelae. This is particularly significant in treatment of short-term exacerbations of the underlying disease or condition; in diseases such as asthma, such short-term exacerbations can prove fatal. Similarly, in infection by SARS-CoV-2 virus and its sequelae, such exacerbations can cause extremely serious drops in blood oxygenation requiring immediate and aggressive treatment to avoid fatal consequences, particularly in patients with preexisting conditions such as obesity, diabetes, or hypertension.

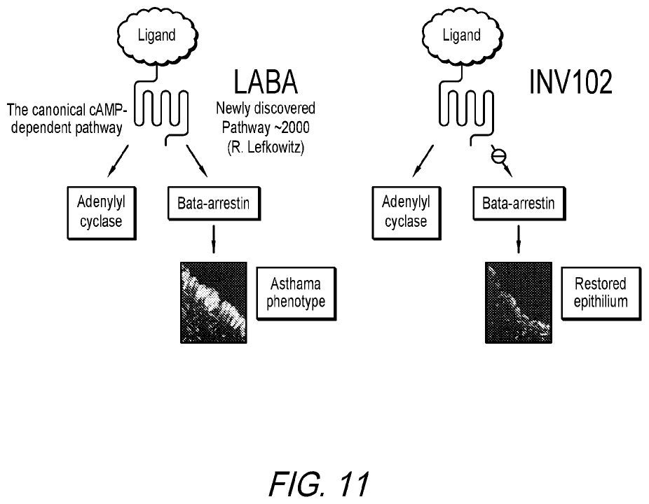

FIG. 11 is a schematic diagram showing the mechanism of action of nadolol as contrasted with the mechanism of action of long-acting β-adrenoceptor agonists (LABA) and that nadolol (“INV102”) reverses epithelial changes via inhibition of the beta-arrestin pathway in 32 airway receptors.

### Example 12

### Nadolol Reduces the Accumulation of the Mucus-Associated Protein Mucin 5AC

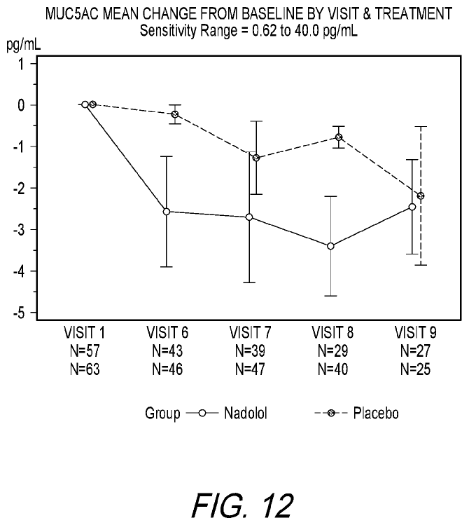

Nadolol reduces the accumulation of the mucus-associated protein mucin 5AC. FIG. 12 is a graph showing the effect of nadolol on the level of mucin 5AC in smokers treated with nadolol versus the results with a placebo. The results are statistically significant with P<0.05. After dosing ended, the phenotype regressed toward the mean, arguing for longer term treatment to enhance and maintain benefit of the administration of the nadolol. As stated above, reduction of this protein can substantially benefit patients with infection by SARS-CoV-2 virus or sequelae of infection by reducing airway obstruction and thereby improving blood oxygenation.

### Example 13

### Nadolol Acts to Block the Beta-Arrestin Pathway

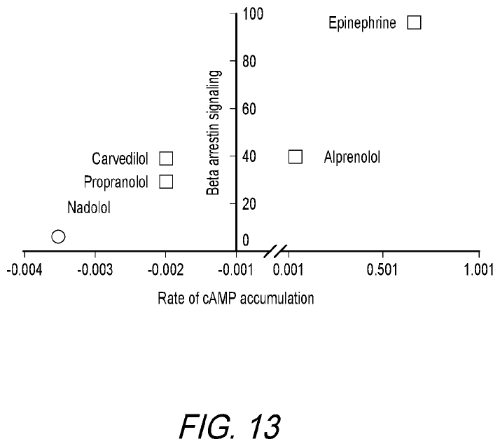

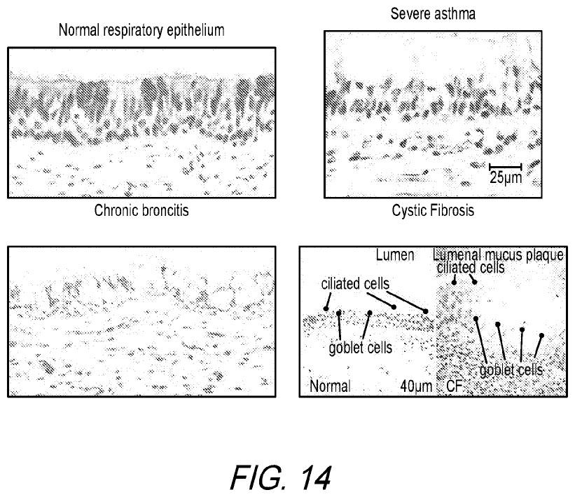

Nadolol acts to block the β-arrestin pathway, which, as stated above, results in greater therapeutic efficacy when β₂-adrenergic agonists are administered to treat diseases and conditions affecting the respiratory tract. In comparison, carvedilol, propranolol, and alprenolol did not block the β-arrestin pathway, as shown in FIG. 13. Beta-blockers are typically associated with adverse mucus production via the β-arrestin pathway; nadolol blocks this pathway. Nadolol, as shown in FIG. 14, is not associated with cAMP accumulation.

### Example 14

### Nadolol Can Restore the Normal State of the Respiratory Epithelium

As shown above, nadolol, through its actions in acting as a beta-adrenergic inverse agonist and inhibiting the activity of the β-arrestin pathway, can restore the normal state of the respiratory epithelium. FIG. 14 is a set of photomicrographs showing the respiratory epithelium in: normal subject without airway disease (upper left); severe asthma (upper right); chronic bronchitis (lower left); and cystic fibrosis (lower right). This leads to the conclusion that infection by SARS-CoV-2 virus or its sequelae can be treated by the administration of nadolol as described above, together with other agents, also as described above, in order to restore the normal state of the respiratory epithelium.

### Advantages of the Invention

The present invention provides improved methods and compositions for treating infection by SARS-CoV-2 virus or its sequelae, focusing on prevention of serious, long-term airway congestion, potentially leading to a substantial drop in blood oxygenation requiring hospitalization and aggressive treatment, as well as on prevention of hyperinflammation associated with the presence of cytokine storms. These methods and compositions avoid the tolerance or tachyphylaxis that is often the consequence of therapy with conventional therapy with agents such as β-adrenergic agonists. The use of inverse agonists, in essence, forces the body to respond by improving its own signaling mechanisms to counter the pulmonary airway disease. Accordingly, compositions and methods that employ inverse agonists have broad potential for treating infection with SARS-CoV-2 virus and its sequelae without the induction of tolerance. This promises superior long-term results in the treatment of SARS-CoV-2 virus and its sequelae without interfering with short-term acute therapy. Methods and compositions according to the present invention are well-tolerated and can be used together with other methods or therapeutic agents for treating infection with SARS-CoV-2 virus and its sequelae. Methods and compositions according to the present invention can also be used together with vaccines against the SARS-CoV-2 virus.

As used herein in the specification and claims, the transitional phrase “comprising” and equivalent language also encompasses the transitional phrases “consisting essentially of” and “consisting of” with respect to the scope of any claims presented herein, unless the narrower transitional phrases are explicitly excluded.

Methods according to the present invention possess industrial applicability for the preparation of a medicament for the treatment of pulmonary airway diseases, in particular, SARS-CoV-2 and its sequelae. Methods according to the present invention also possess industrial applicability for use in treating SARS-CoV-2 and its sequelae. Compositions according to the present invention possess industrial applicability as pharmaceutical compositions, particularly for the treatment of SARS-CoV-2 and its sequelae.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug. Methods as described herein also encompass use of the compounds, combinations of compounds, or compositions described herein for the treatment of the diseases or conditions described herein, as well as methods for the preparation of a medicament for the treatment of the diseases or conditions described herein.

The inventions illustratively described herein can suitably be practiced in the absence of any element or elements, limitation or limitations, not specifically disclosed herein. Thus, for example, the terms “comprising,” “including,” “containing,” and equivalent or similar terminology shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group, and that unless excluded, such an individual member or subgroup of members is within the scope of the claimed invention. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

1. A method for treatment of infection with SARS-CoV-2 virus or sequelae of infection with SARS-CoV-2 virus in a subject suffering from SARS-CoV-2 virus comprising administration of a therapeutically effective quantity of nadolol to inhibit the β-arrestin pathway to treat the infection with SARS-CoV-2 virus or sequelae of infection with SARS-CoV-2 virus.

2.-3. (canceled)

4. The method of claim 1 wherein the method exerts a therapeutic effect selected from: a reduction in pulmonary airway constriction hyperresponsiveness; an upregulation of pulmonary β₂-adrenergic receptors; a reversal of mucous metaplasia and mucus cell metaplasia; and a blockage of the ability of the SARS-CoV-2 virus to infect cells via the ACE2 receptor.

5.-8. (canceled)

9. The method of claim 1 wherein the nadolol is administered by a route selected from the group consisting of oral, sustained-release oral, parenteral, sublingual, buccal, administration by insufflation, and administration by inhalation.

10.-23. (canceled)

24. The method of claim 1 wherein the method further comprises administration of a therapeutically effective quantity of an additional agent, wherein the additional agent is selected from the group consisting of:
(a) a β₂-selective adrenergic agonist selected from the group consisting of abediterol, arformoterol, bambuterol, bitolterol, broxaterol, carbuterol, carmoterol, clenbuterol, colterol, dobutamine, fenoterol, formoterol, indicaterol, isoprenaline, isoxsuprine, levabuterol, mabuterol, metaproterenol, methoxyphenamine, navafenterol, olodaterol, pirbuterol, procaterol, ritodrine, salbutamol, salmeterol, terbutaline, vilanterol, and zilpaterol, and the salts, solvates, and prodrugs thereof;
(b) a corticosteroid selected from the group consisting of beclomethasone, budesonide, ciclesonide, deflazacort, flunisolide, fluticasone, methylprednisolone, mometasone, prednisolone, prednisone, betamethasone, dexamethasone, triamcinolone, acrocinonide, alclometasone, amcinafal, amcinafide, amebucort, amelometasone, benzodrocortisone, butixocort, chloroprednisone, ciclometasone, ciprocinonide, clobetasol, clorcotolone, cormetasone, cortobenzolone, deprodone, descinolone, dexbudesonide, dimesone, domoprednate, doxibetasol, drocinonide, endrisone, etiprednol, fluazacort, flucorolone, flumetasone, flumoxonide, fluocinolone, fluocinonide, fluocortin, fluocortolone, 9α-fluorocortisone, fluorometholone, flupamesone, fluperolone, fluprednidene, halcinonide, halocortolone, halopredone, halocortamate, hydrocortisone, icometasone, isoflupredone, isoprednidene, itrocinonide, locicortolone, mazipredone, meclorisone, meprednisone, naflocort, nicocortonide, nivacortol, oxisopred, paramethasone, prednylidene, procinonide, resocortol, rofleponide, ticabesone, timobesone, tipredane, tixocortol, tralonide, triclonide, and the salts, solvates, and prodrugs thereof;
(c) an anticholinergic drug selected from the group consisting of ipratropium bromide, tiotropium bromide, oxitropium bromide, aclidinium bromide, glycopyrronium bromide, umeclidinium bromide, and abediterol, and the salts, solvates, and prodrugs thereof;
(d) a xanthine compound selected from the group consisting of theophylline, extended-release theophylline, aminophylline, theobromine, enprofylline, diprophylline, isbufylline, choline theophyllinate, albifylline, arofylline, bamifylline, caffeine, 8-chlorotheophylline, diprophylline, doxofylline, enprofylline, etamiphylline, furafylline, 3-isobutyl-1-methylxanthine, IBMX (1-methyl-3-(2-methylpropyl)-7H-purine-2,6-dione), MRS-1706 (N-(4-acetylphenyl)-2-[4-(2,3,6,7-tetrahydro-2,6-dioxo-1,3-dipropyl-1H-purin-8-yl)phenoxy]acetamide), ambuphylline, proxyphylline, and xanthinol, and the salts, solvates, and prodrugs thereof;
(e) an anti-IgE antibody;
(f) a leukotriene antagonist selected from the group consisting of ablukast, tipelukast, montelukast, pranlukast, and zafirlukast, and the salts, solvates, and prodrugs thereof;
(g) a phosphodiesterase IV inhibitor selected from the group consisting of apremilast, atizoram, filaminast, mufemilast, roflumilast, cilomilast, piclamilast, and ibudilast, and the salts, solvates, and prodrugs thereof;
(h) a 5-lipoxygenase inhibitor selected from the group consisting of meclofenamate sodium, zileuton, and fenleuton, and the salts, solvates, and prodrugs thereof;
(i) a mast cell stabilizer selected from the group consisting of bepotastine, alcaftadine, azelastine, cromoglicic acid, ketotifen, lodoxamide, nedocromil, olopatadine, and pemirolast, and the salts, solvates, and prodrugs thereof;
(j) a biological selected from the group consisting of an anti-IL-4 antibody, an anti-IL-13 antibody, an inhibitor of both IL-4 and IL-13, an anti-IL-5 antibody, and an anti-IL-8 antibody; and
(k) a MAPK inhibitor selected from the group consisting of PD 184352 (2-(2-chloro-4-iodoanilino)-N-(cyclopropylmethoxy)-3,4-difluorobenzamide), neflamapimod, SB 202190 (4-[4-(4-fluorophenyl)-5-pyridin-4-yl-1H-imidazol-2-yl]phenol hydrochloride), anisomycin, PD 98059 (2-(2-amino-3-methoxyphenyl)chromen-4-one), SB 203580 (4-[4-(4-fluorophenyl)-2-(4-methylsulfinylphenyl)-1H-imidazol-5-yl]pyridine hydrochloride), U0126 (1,4-diamino-2,3-dicyano-1,4-bis(2-aminophenylthio)butadiene), AG 126 (2-[(3-hydroxy-4-nitrophenyl)methylidene]propanedinitrile), apigenin, 5-iodotubercidin, olomoucine, iso-olomoucine, N⁹-isopropyl olomoucine, PD 169316 (4-[4-(4-fluorophenyl)-2-(4-nitrophenyl)-1H-imidazol-5-yl]pyridine), SB 202474 (4-[5-ethyl-2-(4-methoxyphenyl)-1H-imidazol-4-yl]pyridine), SB 202190 (4-[4-(4-fluorophenyl)-5-pyridin-4-yl-1H-imidazol-2-yl]phenol hydrochloride), SB 202474 (4-[5-ethyl-2-(4-methoxyphenyl)-1H-imidazol-4-yl]pyridine dihydrochloride), adezmapimod, SB 203580 sulfone (4-[4-(4-fluorophenyl)-2-(4-methylsulfonylphenyl)-1H-imidazol-5-yl]pyridine), SB 220025 (4-[5-(4-fluorophenyl)-3-piperidin-4-ylimidazol-4-yl]pyrimidin-2-amine), SC 68376 (2-methyl-4-phenyl-5-pyridin-4-yl-1,3-oxazole), SKF-86002 (6-(4-fluorophenyl)-5-pyridin-4-yl-2,3-dihydroimidazo[2,1-b][1,3]thiazole), U0124 ((2Z,3Z)-2,3-bis[amino(methylsulfanyl)methylidene]butanedinitrile), ZM 336372 (3-(dimethylamino)-N-[3-[(4-hydroxybenzoyl)amino]-4-methylphenyl]benzamide), doramapimod, ralimetinib dimesylate, PH-797804 (3-[3-bromo-4-[(2,4-difluorophenyl)methoxy]-6-methyl-2-oxopyridin-1-yl]-N,4-dimethylbenzamide), VX-702 (6-(N-carbamoyl-2,6-difluoroanilino)-2-(2,4-difluorophenyl)pyridine-3-carboxamide) losmapimod, SB 269063 (4-[4-(4-fluorophenyl)-5-(2-methoxypyrimidin-4-yl)imidazol-1-yl]cyclohexan-1-ol), BMS-582949 (4-[5-(cyclopropylcarbamoyl)-2-methylanilino]-5-methyl-N-propylpyrrolo[2,1-f][1,2,4]triazine-6-carboxamide), skepinone-L, TAK-715 (N-[4-[2-ethyl-4-(3-methylphenyl)-1,3-thiazol-5-yl]pyridin-2-yl]benzamide), pamapimod, pexmetinib, SD 0006 (1-[4-[3-(4-chlorophenyl)-4-pyrimidin-4-yl-1H-pyrazol-5-yl]piperidin-1-yl]-2-hydroxyethanone), and R1487 (6-(2,4-difluorophenoxy)-8-methyl-2-(oxan-4-ylamino)pyrido[2,3-d]pyrimidin-7-one hydrochloride), and the salts, solvates, and prodrugs thereof.

25.-66. (canceled)

67. The method of claim 1 wherein the method further comprises administration of a therapeutically effective quantity of an arrestin-2 inhibitor, wherein the arrestin-2 inhibitor is selected from the group consisting of:
(a) a protein fragment of arrestin-2;
(b) a compound of Formula (A-1): 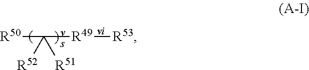
wherein:
(1) v and vi designate the particular bonds indicated in Formula (A-I);
(2) R⁴⁹ is selected from Formulas (A-I(a)), (A-I(b)), (A-I(c)), and (A-I(d)): 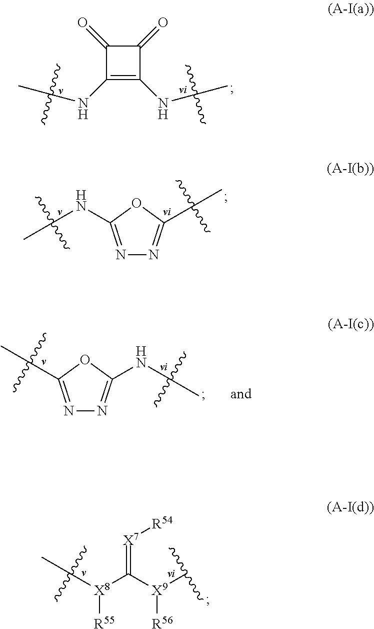
wherein X⁷, X⁸, and X⁹ are each independently O, N, or S;
(2) R⁵⁴, R⁵⁵, and R⁵⁶ are each independently H, cyano, amino, or a substituted or unsubstituted alkyl, alkanoyl, alkanoyloxy, or aryl group when X⁷, X⁸, or X⁹ are respectively N and are absent when X⁷, X⁸, or X⁹ are respectively O or S;
(3) R⁵⁰ is a substituted or unsubstituted aryl or heteroaryl group;
(4) R⁵¹ and R⁵² are each independently H or a substituted or unsubstituted alkyl group, or R⁵¹ and R⁵² together form a 3- or 4-membered cycloalkyl ring;
(5) R⁵³ is a substituted aryl group where one and only one of the substituents is a moiety of Formula (A-I(e)): 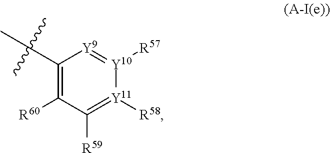
(i) wherein one of R⁵⁷ or R⁵⁸ is a moiety of Formula (A-I(f)): 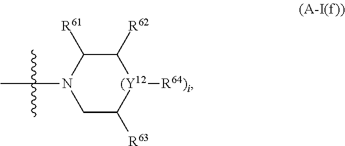
and the other is H, azido, trifluoromethyldiazirido, isocyano, isothiocyano, pentafluorosulfanyl, or a substituted or unsubstituted alkyl, alkanoyl, alkanoyloxy, aryloyl, or aryloyloxy group;
(ii) R⁵⁹ and R⁶⁰ are each independently H, halo, azido, trifluoromethyldiazirido, isocyano, isothiocyano, pentafluorosulfuryl, or a substituted or unsubstituted alkyl, alkanoyl, alkanoyloxy, aryloyl, or aryloyloxy group;
(iii) Y⁹ is CH;
(iv) Y¹⁰ and Y¹¹ are each independently C or N, provided that when Y¹⁰ or Y¹¹ is N then R⁵⁷ or R⁵⁸ respectively is absent;
(v) Y¹² is CH, N, O, S, S(O), or S(O)₂;
(vi) R⁶¹, R⁶², and R⁶³ are each independently H, azido, trifluoromethyldiazirido, isocyano, isothiocyano, or a substituted or unsubstituted alkyl group;
(vii) R⁶⁴ is H or a substituted or unsubstituted alkyl group when Y¹² is CH or N and is absent when Y¹² is O, S, S(O), or S(O)₂; and
(viii) t is 0 or 1; and
(6) s is 0 or 1;
(c) an omega-3 fatty acid selected from the group consisting of DHA (docosahexaenoic acid), EPA (eicosapentaenoic acid), HTA (hexadecatrienoic acid), ALA (α-linolenic acid), SDA (stearidonic acid), ETE (eicosatrienoic acid), ETA (eicosatetraenoic acid), HPA (heneicosapentaenoic acid), DPA (docosapentaenoic acid), tetracosapentaenoic acid, and tetracosahexaenoic acid;
(d) a CXCR2 inhibitor selected from the group consisting of SB225002 (N-(2-bromophenyl)-N′-(2-hydroxy-4-nitrophenyl)urea), AZD5069 (N-(2-((2,3-difluorobenzyl)thio)-6-(((2R,3S)-3,4-dihydroxybutan-2-yl)oxy)pyrimidin-4-yl)azetidine-1-sulfonamide); SB265610 (1-(2-bromophenyl)-3-(4-cyano-1H-benzo[d][1,2,3]triazol-7-yl)urea); navarixin; danirixin; CXCR2-IN-1 (1-(2-chloro-3-fluorophenyl)-3-[4-chloro-2-hydroxy-3-(1-methypiperidin-4-yl)sulfonyphenyl]urea); SRT3109 (N-(2-((2,3-difluorobenzyl)thio)-6-((3,4-dihydroxybutan-2-yl)amino)pyrimidin-4-yl)azetidine-1-sulfonamide); and SRT3190 (N-[2-[(2,3-difluorophenyl)methylsulfanyl]-6-[[(2S,3R)-3,4-dihydroxybutan-2-yl]amino]pyrimidin-4-yl]azetidine-1-sulfonamide);
(e) MyD88 inhibitor selected from the group consisting of ST2825 ((4R,7R,8aR)-1′-[2-[4-[[2-(2,4-dichlorophenoxy)acetyl]amino]phenyl]acetyl]-6-oxospiro[3,4,8,8a-tetrahydro-2H-pyrrolo[2,1-b][1,3]thiazine-7,2′-pyrrolidine]-4-carboxamide) and T6167923 (4-(3-bromophenyl)sulfonyl-N-(1-thiophen-2-ylethyl)piperazine-1-carboxamide); and
(f) a MD2 inhibitor that is L48H37 ((3E,5E)-1-ethyl-3,5-bis[(2,3,4-trimethoxyphenyl)methylidene]piperidin-4-one).

68.-77. (canceled)

78. The method of claim 1 wherein the method further comprises administration of a therapeutically effective quantity of an inhibitor of a GRK.

79.-87. (canceled)

88. The method of claim 1 wherein the method further comprises administration of a therapeutically effective quantity of an arrestin-2 inhibitor selected from the group consisting of:
(a) barbadin;
(b) inositol hexaphosphate;
(c) ML339 (N-[(1S,5R)-9-[2-(2-chloroanilino)-2-oxoethyl]-9-azabicyclo[3.3.1]nonan-3-yl]-3,4,5-trimethoxybenzamide);
(d) an inhibitor of protein kinase A selected from the group consisting of: (i) H89 (N-[2-[[3-(4-bromophenyl)-2-propenyl]amino]ethyl]-5-isoquinolinesulfonamide dihydrochloride); (ii) N—(ω-undecylenoyl)phenylalanine; (iii) 3′,5′-cyclic monophosphorothioate-R; (iv) H-7 (5-(2-methylpiperazin-1-yl)sulfonylisoquinoline dihydrochloride); (v) H-9 (N-(2-aminoethyl)-5-isoquinolinesulfonamide; (vi) 6-22 amide; and (vii) a protein kinase A inhibitor selected from the group consisting of: fasudil; N-[2-(phosphorylated bromonitroarginylamino)ethyl]-5-isoquinoline sulfonamide; 1-(5-guinolinesulfonyl)piperazine; 4-cyano-3-methylisoquinoline; acetamido-4-cyano-3-methylisoguinoline; 8-bromo-2-monoacyladenosine-3,5-cyclic monophosphorothioate; adenosine 3,5-cyclic monophosphorothioate; 2-O-monobutyl-cyclic adenosine monophosphate; 8-chloro-cyclic adenosine monophosphate; N-[2-(cinnamoylamino acid)]-5-isoquinolinone; reverse phase-8-hexylamino adenosine 3,5-monophosphorothioate; reverse phase-8-piperidinyladenosine-cyclic adenosine monophosphate; reverse phase-adenosine 3,5-cyclic monophosphorothioate; 5-iodotuberculin; 8-hydroxyadenosine-3,5-monophosphorothioate; calphostin C; daphnetin; reverse phase-8-chlorophenyl-cyclic adenosine monophosphate; reverse phase-cyclic adenosine monophosphate; reverse phase-8-Br-cyclic adenosine monophosphate; 1-(5-isoquinolinesulfonyl)-2-methypiperidine; 8-hydroxyadenosine-3′,5′-monophosphate; 8-hexylaminoadenosine-3′,5′-monophosphate; and reverse phase-adenosine 3′,5′-cyclic monophosphate;
(d) a phospholipase C inhibitor selected from the group consisting of: sodium aristolochate; D609 (sodium tricyclodecan-9-yl xanthogenate); D-erythro-dihydrosphingosine; U-73122 (1-(6-((17β-3-methoxyestra-1,3,5(10)-trien-17-yl)amino)hexyl)-1H-pyrrole-2,5-dione); pyrrolidinethiocarbamate; neomycin sulfate; thielavin B; edelfosine; heterocyclyl-substituted anilino phospholipase C inhibitors; DCIC (3,4-dichloroisocoumarin); and calporoside or derivatives of calporoside.

89.-94. (canceled)

95. The method of claim 1 wherein the method further comprises administration of a therapeutically effective quantity of an additional agent, wherein the additional agent is an agent for treating a SARS-CoV-2 infection, wherein the agent for treating a SARS-CoV-2 infection is selected from the group consisting of a monoclonal antibody, a small molecule, and a vaccine against SARS-CoV-2.

96.-109. (canceled)

110. The method of claim 1 wherein the method further comprises administration of a therapeutically effective quantity of an additional agent, wherein the additional agent is selected from the group consisting of:
(a) an IL-6 inhibitor selected from the group consisting of sarilumab and siltuximab;
(b) an IL-1 inhibitor selected from the group consisting of anakinra and canakinumab;
(c) a JAK pathway inhibitor that is tofacitinib;
(d) a TNF-α inhibitor that is infliximab;
(e) an IL-17 inhibitor that is secukinumab;
(f) an agent that inhibits MUC5AC selected from the group consisting of niclosamide and carbocisteine;
(g) an EGFR tyrosine kinase inhibitor selected from the group consisting of gefitinib, afatinib, brigatinib, erlotinib, lapatinib, neratinib, olmutinib, osimertinib, rociletinib, and vandetanib;
(h) a p38 mitogen-activated protein kinase inhibitor that is acumapimod;
(i) anti-IDO antibody, clone 10.1;
(j) a statin selected from the group consisting of simvastatin, atorvastatin, fluvastatin, lovastatin, pitavastatin, pravastatin, and rosuvastatin;
(k) BIO-11006 acetate; and
(l) an SGLT2 inhibitor selected from the group consisting of canagliflozin, dapagliflozin, empagliflozin, ertugliflozin, ipragliflozin, luseogliflozin, remogliflozin etabonate, sergliflozin etabonate, sotagliflozin, and tofogliflozin.

111.-134. (canceled)

135. A pharmaceutical composition comprising:
(a) a therapeutically effective quantity of nadolol to inhibit the β-arrestin pathway to treat infection with SARS-CoV-2 virus or sequelae of such infection; and
(b) a pharmaceutically acceptable carrier.

136.-138. (canceled)

139. The pharmaceutical composition of claim 135 wherein administration of the composition exerts a therapeutic effect that is selected from the group consisting of: a reduction in pulmonary airway constriction hyperresponsiveness; an upregulation of pulmonary β₂-adrenergic receptors; an upregulation of pulmonary β₂-adrenergic receptors; increased pulmonary airway relaxation responsiveness to β₂-adrenergic agonist drugs; a reversal of mucous metaplasia and mucus cell hyperplasia; and a blockage of the ability of the SARS-CoV-2 virus to infect cells via the ACE2 receptor.

140.-143. (canceled)

144. The pharmaceutical composition of claim 135 wherein the composition is formulated for administration by a route selected from the group consisting of oral, sustained-release oral, parenteral, sublingual, buccal, administration by insufflation, and administration by inhalation.

145.-150. (canceled)

151. The pharmaceutical composition of claim 135 wherein the quantity of nadolol per unit dose of the composition is selected from the group consisting of 1 mg, 3 mg, 5 mg, 10 mg, 15 mg, 30 mg, 50 mg, and 70 mg per unit dose.

152. The pharmaceutical composition of claim 135 wherein the composition further comprises a therapeutically effective quantity of an additional therapeutic agent, wherein the additional therapeutic agent is selected from the group consisting of;
(a) a β₂-selective adrenergic agonist;
(b) a corticosteroid;
(c) an anticholinergic drug;
(d) a xanthine compound;
(e) an anti-IgE antibody;
(f) a leukotriene antagonist;
(g) a phosphodiesterase IV inhibitor;
(h) a 5-lipoxygenase inhibitor;
(i) a mast cell stabilizer;
(j) a biological;
(k) a MAPK inhibitor;
(l) an arrestin-2 inhibitor;
(m) an agent for treating a SARS-CoV-2 infection that is selected from the group consisting of a monoclonal antibody and a small molecule;
(n) an IL-6 inhibitor;
(o) an IL-1 inhibitor;
(p) a JAK pathway inhibitor;
(q) a TNF-α inhibitor;
(r) an IL-17 inhibitor;
(s) an agent that inhibits MUC5AC;
(t) an EGFR tyrosine kinase inhibitor;
(u) a p38 mitogen-activated protein kinase inhibitor;
(v) anti-IDO antibody, clone 10.1;
(w) a statin;
(y) BIO-11006 acetate; and
(z) an SGLT2 inhibitor.

153.-258. (canceled)

259. The pharmaceutical composition of claim 135 wherein the pharmaceutically acceptable carrier is selected from the group consisting of a solvent, a dispersion medium, a coating, an antibacterial agent, an antifungal agent, an isotonic agent, an absorption delaying agent, a preservative, a sweetening agent for oral administration, a thickening agent, a buffer, a liquid carrier, a wetting, solubilizing, or emulsifying agent; an acidifying agent, an antioxidant, an alkalinizing agent, a carrying agent, a chelating agent, a colorant, a complexing agent, a suspending or viscosity-increasing agent, a flavor or perfume, an oil, a penetration enhancer, a polymer, a stiffening agent, a protein, a carbohydrate, a bulking agent, and a lubricating agent.

260.-278. (canceled)
